(19) United States
(12) Patent Application Publication
(10) Pub. No.: US 2021/0172008 A1
(43) Pub. Date: Jun. 10, 2021

(54) METHODS AND COMPOSITIONS TO IDENTIFY NOVEL CRISPR SYSTEMS

(71) Applicant: LIFEEDIT, INC., Durham, NC (US)

(72) Inventors: Alexandra Briner Crawley, Cary, NC (US); James R. Henriksen, Fort Collins, CO (US); Mark Moore, Durham, NC (US); Rebecca E. Thayer, Morrisville, NC (US)

(73) Assignee: LIFEEDIT, Inc., Durham, NC (US)

(21) Appl. No.: 17/045,053

(22) PCT Filed: Apr. 3, 2019
(86) PCT No.: PCT/US2019/025519

§ 371 Date: Oct. 2, 2020

Related U.S. Application Data
(60) Provisional application No. 62/652,642, filed on Apr. 4, 2018.

(51) Int. Cl. C12Q 1/6813; C12N 9/22; C12N 15/113

(52) U.S. Cl. CPC C12Q 1/6813; C12N 2310/20; C12N 15/113; C12N 9/22

(57) ABSTRACT

Compositions and methods for isolating new variants of known clustered regularly-interspaced short palindromic repeat (CRISPR) RNA-guided nuclease (RGN) genes and new CRISPR systems are provided. The methods find use in identifying CRISPR RGN gene variants in complex mixtures. Compositions comprise hybridization baits that hybridize to CRISPR RGN genes of interest in order to selectively enrich variant polynucleotides from complex mixtures. Bait sequences may be specific for a number of CRISPR RGN genes from distinct gene families of interest and may be designed to cover each CRISPR RGN gene of interest by at least 2-fold. Bait pools may also comprise baits for sequences flanking CRISPR RGN genes of interest to allow for the identification of tracrRNAs corresponding to novel CRISPR RGN variants and a complete CRISPR system comprising a CRISPR RGN and its associated guide RNA.

### FIELD OF THE INVENTION

The invention is drawn to high throughput methods of discovery of genes useful for targeted genome editing.

### REFERENCE TO A SEQUENCE LISTING SUBMITTED ELECTRONICALLY AS A TEXT FILE

The instant application contains a Sequence Listing which has been submitted electronically in ASCII format via EFS-Web and is hereby incorporated by reference in its entirety. Said ASCII copy, created on Mar. 29, 2019, is named L1034381010WO_0028_0_SL.txt, and is 41,946 bytes in size.

### BACKGROUND OF THE INVENTION

Targeted genome editing or modification is rapidly becoming an important tool for basic and applied research, with clustered regularly interspaced short palindromic repeat (CRISPR) RNA-guided nucleases (RGNs) showing the most promise due to the ease of altering target specificity by engineering associated guide RNAs. Currently, only three CRISPR RGNs are available commercially and widely used in the literature: Streptococcus pyogenes Cas9, Staphylococcus aureus Cas9, and Fransicella novicida Cpf1. Given the diversity and abundance of microbial genomes, it is likely a large number of CRISPR RGNs have yet to be identified, many of which might exhibit alternate target recognition or improved activity over the three commercially available CRISPR RGNs. Complex samples containing mixed cultures of organisms often contain species that cannot be cultured or present other obstacles to performing traditional methods of gene discovery. Thus, a high throughput method of identifying new CRISPR RGN genes and systems, where up to millions of culturable and non-culturable microbes can be queried simultaneously would be advantageous. Newly identified RNA-guided nucleases can be used to edit genomes through the introduction of a sequence-specific, double-stranded break that is repaired via error-prone non-homologous end-joining (NHEJ) to introduce a mutation at a specific genomic location. Alternatively, heterologous DNA may be introduced into the genomic site via homology-directed repair.

### BRIEF SUMMARY OF THE INVENTION

Compositions and methods for isolating new variants of known clustered regularly interspaced short palindromic repeats (CRISPR) RNA-guided nuclease (RGN) genes are provided. The provided compositions and methods are also useful in identifying a corresponding tracrRNA for new CRISPR RGN variants, and thus can be used to identify new CRISPR systems comprising an RGN and its associated guide RNA. The methods find use in identifying CRISPR RGN genes, and in some embodiments, CRISPR systems, in complex mixtures. Compositions comprise hybridization baits that hybridize to CRISPR RGN genes of interest, and in some embodiments flanking sequences, in order to selectively enrich the polynucleotides of interest from complex mixtures. Bait sequences may be specific for a number of distinct CRISPR RGN genes and may be designed to cover each CRISPR RGN gene of interest, and in some embodiments flanking sequences, by at least 2-fold. Thus, methods disclosed herein are drawn to an oligonucleotide hybridization gene capture approach for identification of new CRISPR RGN genes or CRISPR systems of interest from environmental samples. This approach bypasses the need for labor-intensive microbial strain isolation, permits simultaneous discovery of CRISPR RGN genes and CRISPR systems from multiple families of interest, and increases the potential to discover CRISPR RGN genes and CRISPR systems from low-abundance and unculturable organisms present in complex mixtures of environmental microbes.

### DETAILED DESCRIPTION

Methods for identifying variants of known CRISPR RGN genes, and in some embodiments, their corresponding tracrRNAs, from complex mixtures are provided. The methods use labeled hybridization baits or bait sequences that correspond to a portion of known CRISPR RGN genes, and in some embodiments flanking sequences, to capture similar sequences from complex environmental samples. Once the DNA sequence is captured, subsequent sequencing and analysis can identify variants of the known CRISPR RGN genes and systems in a high throughput manner.

The methods of the invention are capable of identifying and isolating variants of known CRISPR RGN genes and CRISPR systems from a complex sample. By “complex sample” is intended any sample having DNA from more than one species of organism. In specific embodiments, the complex sample is an environmental sample, a biological sample, and/or a metagenomic sample. As used herein, the term “metagenome” or “metagenomic” refers to the collective genomes of all microorganisms present in a given habitat (Handelsman et al., (1998) Chem. Biol. 5: R245-R249; Microbial Metagenomics, Metatranscriptomics, and Metaproteomics. Methods in Enzymology vol. 531 DeLong, ed. (2013)). Environmental samples can be from soil, rivers, ponds, lakes, industrial wastewater, seawater, forests, agricultural lands on which crops are growing or have grown, samples of plants or animals or other organisms associated with microorganisms that may be present within or without the tissues of the plant or animal or other organism, or any other source having biodiversity. In some embodiments, complex samples include metagenomics environmental samples that include the collective genomes of all microorganisms present in an environmental sample. Complex samples also include colonies or cultures of microorganisms that are grown, collected in bulk, and pooled for storage and DNA preparation. For example, colonies can be grown on plates, in bottles, or in other bulk containers and collected. In certain embodiments, complex samples are selected based on expected biodiversity that will allow for identification of variants of known CRISPR RGN genes and systems. In some embodiments, samples can be grown under conditions that allow for the growth of certain types of bacteria. For example, particular samples can be grown under either aerobic or anaerobic growth conditions or grown in media that selects for certain bacteria (e.g., methanol or high salt). Selection for certain species could include growth of environmental samples on defined carbon sources (for example, starch, mannitol, succinate or acetate), antibiotics (for example, cephalothin, vancomycin, polymyxin, kanamycin, neomycin, doxycycline, ampicillin, trimethoprim or sulfonamides), chromogenic substrates (for example, enzyme substrates such as phospholipase substrates, lecithinase substrates, cofactor metabolism substrates, nucleosidase substrates, glucosidase substrates, metalloprotease substrates and the like).

The methods disclosed herein do not require purified samples of single organisms but rather is able to identify novel CRISPR RGN genes and systems directly from uncharacterized mixes of populations of prokaryotic organisms: from soil, from crude samples, and samples that are collected and/or mixed and not subjected to any purification. In this manner, the methods described herein can identify CRISPR RGN genes and systems from unculturable organisms, or those organisms that are difficult to culture.

### I. CRISPR Systems

The presently disclosed methods and compositions are useful for identifying novel CRISPR RGN genes and CRISPR systems.

Clustered regularly interspaced short palindromic repeats (CRISPRs) are found in bacterial and archaea genomes and comprise direct repeats interspaced by short segments of spacer DNA that were obtained from previous exposures to foreign DNA. These CRISPRs are transcribed and processed into CRISPR RNAs (crRNA), each of which comprises a CRISPR repeat sequence and a spacer sequence. A CRISPR array comprises an A-T rich leader sequence followed by the CRISPRs, CRISPR-associated system (cas) genes (including those encoding an RGN) and in some systems, a sequence encoding a trans-activating RNA (tracrRNA) within a particular genomic locus.

As used herein, a “CRISPR system” or “clustered regularly-interspaced short palindromic repeats system” comprises an RNA-guided nuclease (RGN) protein and a respective guide RNA that can bind to the RGN and direct the RGN to a target nucleotide sequence for cleavage. A CRISPR RNA-guided nuclease or RGN refers to a polypeptide that binds to a particular target nucleotide sequence in a sequence-specific manner and is directed to the target nucleotide sequence by a guide RNA molecule that is complexed with the polypeptide and hybridizes with the target nucleotide sequence. Generally, genomic sequences encoding RGNs are located near CRISPRs in the genome and thus are referred to herein as CRISPR RGNs. The RGN identified using the presently disclosed methods and compositions may be an endonuclease or an exonuclease. Although many native RNA-guided nucleases are capable of cleaving target nucleotide sequences upon binding, the presently disclosed methods and compositions can be used to identify RNA-guided nucleases that might be nuclease-dead (i.e., are capable of binding to, but not cleaving, a target nucleotide sequence). RNA-guided nucleases identified by the presently disclosed methods and compositions can cleave a target nucleotide sequence, resulting in a single- or double-stranded break. RNA-guided nucleases only capable of cleaving a single strand of a double-stranded nucleic acid molecule are referred to herein as nickases.

A target nucleotide sequence hybridizes with a guide RNA and is bound by an RNA-guided nuclease associated with the guide RNA. The target nucleotide sequence can then be subsequently cleaved by the RNA-guided nuclease if the protein possesses nuclease activity. The terms “cleave” or “cleavage” refer to the hydrolysis of at least one phosphodiester bond within the backbone of a target nucleotide sequence that can result in either single-stranded or double-stranded breaks within the target nucleotide sequence. A CRISPR RGN or system of interest or a CRISPR RGN or system identified using the presently disclosed methods and compositions can be capable of cleaving a target nucleotide sequence, resulting in staggered breaks or blunt ends. A CRISPR RGN or system of interest or a CRISPR RGN or system identified using the presently disclosed methods and compositions can target RNA or DNA, which can be single-stranded or double-stranded, or RNA:DNA hybrids.

A single organism can comprise multiple CRISPR systems of the same or different types. While the presently disclosed methods and compositions can be used to identify either Class 1 or Class 2 CRISPR systems, Class 2 CRISPR systems are of particular interest given that they comprise a single polypeptide with RGN activity. Class 1 systems, on the other hand, require a complex of proteins for nuclease activity. There are three known types of Class 2 CRISPR systems, Type II, Type V, and Type VI, among which there are multiple subtypes (subtype II-A, II-B, II-C, V-A, V-B, V-C, VI-A, VI-B, and VI-C, among other undefined or putative subtypes). Type II and Type V-B systems require tracrRNA, in addition to crRNA, for RGN activity. In general, Type V-A and VI only require a crRNA. All known Type II and Type V RGNs target double-stranded DNA, whereas all known Type VI RGNs target single-stranded RNA.

The term “guide RNA” refers to a nucleotide sequence having sufficient complementarity with a target nucleotide sequence to hybridize with the target sequence and direct sequence-specific binding of an associated RNA-guided nuclease to the target nucleotide sequence. Thus, a CRISPR RGN's respective guide RNA is one or more RNA molecules (generally, one or two), that can bind to the RGN and guide the RGN to bind to a particular target nucleotide sequence, and in those instances wherein the RGN has nickase or nuclease activity, also cleave the target nucleotide sequence. In some embodiments, the guide RNA comprises a CRISPR RNA (crRNA). In other embodiments, the guide RNA comprises both a crRNA and a trans-activating CRISPR RNA (tracrRNA). Native guide RNAs that comprise both a crRNA and a tracrRNA generally comprise two separate RNA molecules that hybridize to each other through the repeat sequence of the crRNA and the anti-repeat sequence of the tracrRNA.

Native direct repeat sequences within a CRISPR array generally range in length from 28 to 37 base pairs, although the length can vary between about 23 bp to about 55 bp. Spacer sequences within a CRISPR array generally range from about 32 to about 38 bp in length, although the length can be between about 21 bp to about 72 bp. Each CRISPR array generally comprises less than 50 units of the CRISPR repeat-spacer sequence. The CRISPRs are transcribed as part of a long transcript termed the primary CRISPR transcript, which comprises much of the CRISPR array. The primary CRISPR transcript is cleaved by cas proteins to produce crRNAs or in some cases, to produce pre-crRNAs that are further processed by additional cas proteins into mature crRNAs. Mature crRNAs comprise a spacer sequence and a CRISPR repeat sequence. In some embodiments in which pre-crRNAs are processed into mature crRNAs, maturation involves the removal of about one to about six or more 5′, 3′, or 5′ and 3′ nucleotides. For the purposes of genome editing or targeting a particular target nucleotide sequence of interest, these nucleotides that are removed during maturation of the pre-crRNA molecule are not necessary for generating or designing a guide RNA.

A CRISPR RNA (crRNA) comprises a spacer sequence and a CRISPR repeat sequence. The “spacer sequence” when referring to native crRNAs is the nucleotide sequence that directly hybridizes with a protospacer on a foreign DNA. A spacer sequence can also be engineered to be fully or partially complementary to a target nucleotide sequence of interest for the use of genome editing or targeting a particular genomic locus. The spacer sequence of engineered crRNAs can be about 8 to about 30 nucleotides in length, including about 8, about 9, about 10, about 11, about 12, about 13, about 14, about 15, about 16, about 17, about 18, about 19, about 20, about 21, about 22, about 23, about 24, about 25, about 26, about 27, about 28, about 29, and about 30 nucleotides. In some embodiments, the spacer sequence of an engineered crRNA is about 10 to about 26 nucleotides in length, or about 12 to about 30 nucleotides in length. The CRISPR repeat sequence comprises a nucleotide sequence that comprises a region with sufficient complementarity to hybridize to a tracrRNA. The CRISPR repeat sequences of native mature crRNAs and engineered crRNAs can range in length from about 8 to about 30 nucleotides in length, including about 8, about 9, about 10, about 11, about 12, about 13, about 14, about 15, about 16, about 17, about 18, about 19, about 20, about 21, about 22, about 23, about 24, about 25, about 26, about 27, about 28, about 29, and about 30 nucleotides.

In some systems, the CRISPR repeat sequence further comprises a region with secondary structure (e.g., stem-loop) or forms secondary structure upon hybridizing with its corresponding tracrRNA. Native coding sequences for crRNAs are generally on the opposite end of a CRISPR array from the RGN-encoding sequence. Given their distance from RGN-encoding sequences on CRISPR arrays, in some embodiments, the presently disclosed methods of using hybridization baits may not be successful in identifying crRNAs. The CRISPR repeat sequence, however, can be deduced after the identification of the anti-repeat in a CRISPR RGN's tracrRNA, as described elsewhere herein.

In those CRISPR systems that further comprise a tracrRNA, the native tracrRNA is transcribed from the CRISPR array. A tracrRNA molecule comprises a nucleotide sequence comprising a region that has sufficient complementarity to hybridize to a CRISPR repeat sequence, which is referred to herein as the anti-repeat region. In some systems, the tracrRNA molecule further comprises a region with secondary structure (e.g., stem-loop) or forms secondary structure upon hybridizing with its corresponding crRNA. In particular embodiments, the region of the tracrRNA that is fully or partially complementary to a CRISPR repeat sequence is at the 5′ end of the molecule and the 3′ end of the tracrRNA comprises secondary structure. This region of secondary structure generally comprises several hairpin structures, including the nexus hairpin, which is found adjacent to the anti-repeat sequence. The nexus hairpin often has a conserved nucleotide sequence in the base of the hairpin stem, with the motif UNANNC found in the majority of Type IIA nexus hairpins in tracrRNAs. There are often terminal hairpins at the 3′ end of the tracrRNA that can vary in structure and number, but often comprise a GC-rich Rho-independent transcriptional terminator hairpin followed by a string of U's at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

Type IIA guide RNAs also comprise an upper stem, bulge, and lower stem that are created by base-pairing between the CRISPR repeat and the antirepeat of the tracrRNA.

In various embodiments, the anti-repeat region of the tracrRNA that is fully or partially complementary to the CRISPR repeat sequence comprises from about 8 nucleotides to more than about 30 nucleotides. For example, the region of base pairing between the tracrRNA sequence and the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In various embodiments, the entire tracrRNA can comprise from about 60 nucleotides to more than about 140 nucleotides. For example, the tracrRNA can be about 60, about 65, about 70, about 75, about 80, about 85, about 90, about 95, about 100, about 105, about 110, about 115, about 120, about 125, about 130, about 135, about 140, or more nucleotides in length. In particular embodiments, the tracrRNA is about 80 to about 90 nucleotides in length, including about 80, about 81, about 82, about 83, about 84, about 85, about 86, about 87, about 88, about 89, and about 90 nucleotides in length.

The bait sequences described herein can be designed to be complementary to flanking sequences of a known CRISPR RGN of interest such that the coding sequence for a tracrRNA, and thus, the tracrRNA, can be identified.

The sequence and structure of crRNAs and tracrRNAs is often specific for a particular CRISPR system. Thus, in order to identify a complete CRISPR system, the associated crRNA, and in some embodiments, tracrRNA must also be identified using the methods disclosed elsewhere herein or other methods known in the art.

The presently disclosed methods and compositions are useful for identifying variants of CRISPR RGN genes of interest. As used herein, the term “gene” refers to an open reading frame comprising a nucleotide sequence that encodes a polypeptide. In some embodiments, the methods and compositions are utilized to identify a complete CRISPR system (i.e., sequences encoding an RGN and a respective guide RNA, which can comprise both a tracrRNA and a crRNA or a crRNA only).

New variants of known CRISPR RGN genes and systems of interest can be identified using the methods disclosed herein. As used herein, a “CRISPR RGN gene or system of interest” is intended to refer to a known CRISPR RGN gene or system. Known CRISPR RGN genes or systems of interest that can be used in the methods and compositions disclosed herein include, but are not limited to, those listed in Table 1. The sequences and references provided herein are incorporated by reference. It is important to note that these CRISPR RGN genes are provided merely as examples; any CRISPR RGN genes can be used in the practice of the methods and compositions disclosed herein.

The methods disclosed herein can identify variants of known CRISPR RGNs or systems of interest. As used herein, the term “variant” can refer to homologs, orthologs, and paralogs. While the activity of a variant may be altered compared to the CRISPR RGN or system of interest, the variant should retain the functionality of the CRISPR RGN or system of interest. For example, a variant may have increased activity, decreased activity, a different spectrum of activity (e.g., nickase), a different specificity (e.g., altered PAM recognition) or any other alteration in activity when compared to the CRISPR RGN or system of interest.

In general, “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” or “wild type” polynucleotide or polypeptide comprises a naturally occurring nucleotide sequence or amino acid sequence, respectively. For polynucleotides, conservative variants include those sequences that, because of the degeneracy of the genetic code, encode the native amino acid sequence of the CRISPR gene of interest. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the polypeptide of the CRISPR gene of interest. Generally, variants of a particular polynucleotide disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide (e.g., a CRISPR RGN gene of interest) as determined by sequence alignment programs and parameters described elsewhere herein.

Variants of a particular polynucleotide disclosed herein (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides disclosed herein is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

Some known CRISPR RGN genes and polypeptides exhibit relatively low sequence identity across the entire length of the sequences, although particular domains are more conserved. Thus, in some embodiments, the variants (genes or polypeptides) of known CRISPR RGN gene(s) or polypeptide(s) of interest discovered using the presently disclosed methods and compositions may have less than 95%, less than 90%, less than 85%, less than 80%, less than 75%, less than 70%, less than 65%, less than 60%, or less identity to the CRISPR RGN gene(s) or polypeptide(s) of interest. In certain embodiments, the variants (genes or polypeptides) of known CRISPR RGN gene(s) or polypeptide(s) of interest discovered using the presently disclosed methods and compositions may have between 60% and 95%, 65% and 95%, 70% and 95%, 75% and 95%, 80% and 95%, 85% and 95%, 90% and 95% identity to the CRISPR RGN gene(s) or polypeptide(s) of interest.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

The use of the term “polynucleotide” is not intended to limit the present disclosure to polynucleotides comprising DNA. Those of ordinary skill in the art will recognize that polynucleotides can comprise ribonucleotides (RNA) and combinations of ribonucleotides and deoxyribonucleotides. Such deoxyribonucleotides and ribonucleotides include both naturally occurring molecules and synthetic analogues. The polynucleotides disclosed herein also encompass all forms of sequences including, but not limited to, single-stranded forms, double-stranded forms, hairpins, stem-and-loop structures, and the like.

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. O. Dayhoff), pp. 345-352. Natl. Biomed. Res. Found., Washington, D.C. and Henikoff et al. (1992) Proc. Natl. Acad. Sci. USA 89:10915-10919. The BLOSUM62 matrix is often used as a default scoring substitution matrix in sequence alignment protocols. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al. (1997) Nucleic Acids Res. 25:3389-3402, and made available to the public at the National Center for Biotechnology Information Website (www.ncbi.nlm.nih.gov). Optimal alignments, including multiple alignments, can be prepared using, e.g., PSI-BLAST, available through www.ncbi.nlm.nih.gov and described by Altschul et al. (1997) Nucleic Acids Res. 25:3389-3402.

### II. Bait Sequences

The methods and compositions described herein employ bait sequences to capture variants of CRISPR RGN genes or systems of interest from complex samples. As used herein a “bait sequence” or “bait” refers to a polynucleotide that hybridizes to a CRISPR RGN gene or system of interest, or variant thereof. In specific embodiments, bait sequences are single-stranded RNA sequences capable of hybridizing to a fragment of the CRISPR RGN gene or system of interest, or a variant thereof. For example, the RNA bait sequence can be complementary to the DNA sequence of a fragment of the CRISPR RGN gene or system of interest. In some embodiments, the bait sequence is capable of hybridizing to a fragment of the CRISPR RGN gene or system of interest that is at least 50, at least 70, at least 90, at least 100, at least 110, at least 120, at least 130, at least 140, at least 150, at least 170, at least 200, at least 250, at least 400, at least 1000 contiguous nucleotides, and up to the full-length polynucleotide sequence of the CRISPR RGN gene or system of interest. The baits can be contiguous or sequential RNA or DNA sequences. In one embodiment, bait sequences are RNA sequences. RNA sequences cannot self-anneal and work to drive the hybridization.

The bait sequence can be capable of hybridizing to a fragment of the CRISPR RGN gene of interest or a flanking region or a combination of both. A flanking region of a CRISPR RGN gene of interest comprises sequences that are 5′ (i.e., upstream), 3′ (i.e., downstream), or both 5′ and 3′ to the CRISPR RGN gene of interest of sufficient length to allow for the identification of a tracrRNA-coding sequence, which in turn, can be used to determine the tracrRNA sequence by determining the sequence encoded by the tracrRNA-coding sequence. In some embodiments, the flanking regions of a CRISPR RGN gene of interest to which bait sequences are designed are at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250 nucleotides or more 5′, 3′ or both 5′ and 3′ from the CRISPR RGN gene of interest. In certain embodiments, the flanking regions of a CRISPR RGN gene of interest to which bait sequences are designed are about 100 to about 250 or about 150 to about 200 nucleotides 5′, 3′ or both 5′ and 3′ from the CRISPR RGN gene of interest. In specific embodiments, the flanking regions of a CRISPR RGN gene of interest to which bait sequences are designed are about 180 nucleotides 5′, 3′ or both 5′ and 3′ from the CRISPR RGN gene of interest.

In specific embodiments, baits are at least 50, at least 70, at least 90, at least 100, at least 110, at least 120, at least 130, at least 140, at least 150, at least 170, at least 200, or at least 250 contiguous polynucleotides. For example, the bait sequence can be 50-200 nt, 70-150 nt, 100-140 nt, or 110-130 nt in length. In particular embodiments, the bait comprises about 120 nucleotides. The baits can be labeled with any detectable label in order to detect and/or capture the first hybridization complex comprised of a bait sequence hybridized to a fragment of a variant of the CRISPR RGN gene of interest or flanking sequence, or a combination of both. In certain embodiments, the bait sequences are labeled with biotin, a hapten, or an affinity tag or the bait sequences are generated using biotinylated primers, e.g., where the baits are generated by nick-translation labeling of purified target organism DNA with biotinylated deoxynucleotides. In cases where the bait sequences are biotinylated, the target DNA can be captured using a binding partner (e.g., streptavidin molecule) attached to a solid phase. In specific embodiments, the baits are biotinylated RNA baits of about 120 nt in length. Alternatively, antibodies specific for the RNA-DNA hybrid can be used (see, for example, WO2013164319 A1). The baits may include adapter oligonucleotides suitable for PCR amplification, sequencing, or RNA transcription. The baits may include an RNA promoter or are RNA molecules prepared from DNA containing an RNA promoter (e.g., a T7 RNA promoter). The baits can be chemically synthesized or are alternatively transcribed from DNA templates in vitro or in vivo using any method known in the art. The baits can be isolated such that the bait pool is substantially or essentially free from chemical precursors, etc. The baits can be conjugated to a detectable label using any method known in the art. In particular embodiments, the baits are produced using Agilent SureSelect technology, or similar technology from NimbleGen (SeqCap EZ), Mycroarray (MYbaits), Integrated DNA Technologies (XGen), and LC Sciences (OligoMix).

In some embodiments, the bait pool comprises baits that are designed to 16S DNA sequences, or any other phylogenetically differential sequence, in order to capture sufficient portions of the 16S DNA to estimate the distribution of bacterial genera present in the sample.

The bait sequences span substantially the entire sequence of the known CRISPR RGN gene and in some embodiments, flanking sequences. In some embodiments, the bait sequences are overlapping bait sequences. As used herein, “overlapping bait sequences” or “overlapping” refers to fragments of the CRISPR RGN gene of interest and in some embodiments, flanking sequences that are represented in more than one bait sequence. For example, any given 120 nt segment of a CRISPR RGN gene of interest, and in some embodiments, flanking sequences can be represented by a bait sequence having a region complementary to nucleotides 1-60 of the fragment, another bait sequence having a region complementary to nucleotides 61-120 of the fragment, and a third bait sequence complementary to nucleotides 1-120. In some embodiments, at least 10, at least 30, at least 60, at least 90, or at least 120 nucleotides of each overlapping bait overlap with at least one other overlapping bait. In this manner, each nucleotide of a given CRISPR RGN gene of interest and in some embodiments, its flanking sequences, can be represented in at least 2 baits, which is referred to herein as being covered by at least 2× tiling. Accordingly, the method described herein can use baits or labeled baits described herein that cover any CRISPR RGN gene of interest, and in some embodiments, its flanking sequences, by at least 2× or at least 3× tiling.

Baits for multiple CRISPR RGN genes of interest, and in some embodiments flanking sequences, can be used concurrently to hybridize with sample DNA prepared from a complex mixture. For example, if a given complex sample is to be screened for variants of multiple CRISPR RGN genes or systems of interest, baits designed to each CRISPR RGN gene of interest, and in some embodiments, flanking sequences, can be combined in a bait pool prior to, or at the time of, mixing with prepared sample DNA. Accordingly, as used herein, a “bait pool” or “bait pools” refers to a mixture of baits designed to be specific for different fragments of an individual CRISPR RGN gene or system of interest and/or a mixture of baits designed to be specific for different CRISPR RGN genes or systems of interest. “Distinct baits” refers to baits that are designed to be specific for different, or distinct, fragments of CRISPR RGN genes or systems of interest. In some embodiments, a bait pool comprises at least 1,000, at least 5,000, at least 10,000, at least 20,000, at least 30,000, at least 40,000, at least 50,000 or more distinct baits.

Accordingly, in some embodiments, a method for preparing an RNA bait pool for the identification of CRISPR RGN genes or systems of interest is provided. The method comprises identifying overlapping fragments of a DNA sequence of at least one CRISPR RGN gene of interest, wherein the overlapping fragments span the entire DNA sequence of the CRISPR RGN gene of interest, and in some embodiments flanking sequences, and synthesizing RNA baits complementary to the DNA sequence fragments, labeling the RNA baits with a detectable label, and combining the labeled RNA baits to form the RNA bait pool.

A given RNA bait pool can be specific for at least 1, at least 2, at least 10, at least 50, at least 100, at least 150, at least 200, at least 250, at least 300, at least 500, at least 750, at least 800, at least 900, at least 1,000, at least 1,500, at least 3,000, at least 5,000, at least 10,000, at least 15,000, at least 20,000, at least 30,000, at least 40,000, at least 50,000, at least 55,000, at least 60,000, or any other number of CRISPR RGN genes or systems of interest. As used herein, a bait that is specific for a CRISPR RGN gene or system of interest is designed to hybridize to the CRISPR RGN gene of interest, or in some embodiments flanking sequences or a combination of both. A bait can be specific for more than one CRISPR RGN gene or system of interest. In specific embodiments, the sequences of the baits are designed to correspond to CRISPR RGN genes or systems of interest using software tools such as Nimble Design (NimbleGen; Roche).

### III. Methods for Identifying Variants of CRISPR RGN Genes or Systems of Interest

Methods of the invention include preparation of bait sequences, preparation of complex mixture libraries, hybridization selection, sequencing, and analysis. Such methods are set forth in the experimental section in more detail. Additionally, see NucleoSpin® Soil User Manual, Rev. 03, U.S. Publication No. 20130230857; Gnirke et al. (2009) Nature Biotechnology 27:182-189; SureSelect^{XT} Target Enrichment System for Illumina Paired-End Sequencing Library Protocol, Version 1.6; NimbleGen SeqCap EZ Library SR User's Guide, Version 4.3; and NimbleGen SeqCap EZ Library LR User's Guide, Version 2.0, each of which is herein incorporated by reference in its entirety.

Methods of preparing complex samples include fractionation and extraction of environmental samples comprising soil, rivers, ponds, lakes, industrial wastewater, seawater, forests, agricultural lands on which crops are growing or have grown, or any other source having biodiversity. Fractionation can include filtration and/or centrifugation to preferentially isolate microorganisms. In some embodiments, complex samples are selected based on expected biodiversity that will allow for identification of CRISPR RGN genes or systems. Further methods of preparing complex samples include colonies or cultures of microorganisms that are grown, collected in bulk, and pooled for storage and DNA preparation. In certain embodiments, complex samples are subjected to heat treatment or pasteurization to enrich for microbial spores that are resistant to heating. In some embodiments, the colonies or cultures are grown in media that enrich for specific types of microbes or microbes having specific structural or functional properties, such as cell wall composition, resistance to an antibiotic or other compound, or ability to grow on a specific nutrient mix or specific compound as a source of an essential element, such as carbon, nitrogen, phosphorus, or potassium.

In order to provide sample DNA for hybridization to baits as described elsewhere herein, the sample DNA must be prepared for hybridization. Preparing DNA from a complex sample for hybridization refers to any process wherein DNA from the sample is extracted and reduced in size sufficient for hybridization, herein referred to as fragmentation. For example, DNA can be extracted from any complex sample directly, or by isolating individual organisms from the complex sample prior to DNA isolation. In some embodiments, sample DNA is isolated from a pure culture or a mixed culture of microorganisms. DNA can also be extracted directly from the environmental sample. DNA can be isolated by any method commonly known in the art for isolation of DNA from environmental or biological samples (see, e.g. Schneegurt et al. (2003) Current Issues in Molecular Biology 5:1-8; Zhou et al. (1996) Applied and Environmental Microbiology 62:316-322), including, but not limited to, the NucleoSpin Soil genomic DNA preparation kit (Macherey-Nagel GmbH & Co., distributed in the US by Clontech). In one embodiment, extracted DNA can be enriched for any desired source of sample DNA. For example, extracted DNA can be enriched for prokaryotic DNA by amplification. As used herein, the term “enrich” or “enriched” refers to the process of increasing the concentration of a specific target DNA population. For example, DNA can be enriched by amplification, such as by PCR, such that the target DNA population is increased about 1.5-fold, about 2-fold, about 3-fold, about 5-fold, about 10-fold, about 15-fold, about 30-fold, about 50-fold, or about 100-fold. In certain embodiments, sample DNA is enriched by using 16S amplification.

In some embodiments, after DNA is extracted from a complex sample, the extracted DNA is prepared for hybridization by fragmentation (e.g., by shearing) and/or end-labeling. End-labeling can use any end labels that are suitable for indexing, sequencing, or PCR amplification of the DNA. The fragmented sample DNA may be about 100-1000, 100-500, 125-400, 150-300, 200-2000, 100-3000, at least 100, at least 150, at least 200, at least 250, at least 300, or about 350 nucleotides in length. The detectable label may be, for example, biotin, a hapten, or an affinity tag. Thus, in certain embodiments, sample DNA is sheared and the ends of the sheared DNA fragments are repaired to yield blunt-ended fragments with 5′-phosphorylated ends. Sample DNA can further have a 3′-dA overhang prior to ligation to indexing-specific adaptors. Such ligated DNA can be purified and amplified using PCR in order to yield the prepared sample DNA for hybridization. In other embodiments, the sample DNA is prepared for hybridization by shearing, adaptor ligation, amplification, and purification.

In some embodiments, RNA is prepared from complex samples. RNA isolated from complex samples contains genes expressed by the organisms or groups of organisms in a particular environment, which can have relevance to the physiological state of the organism(s) in that environment, and can provide information about what biochemical pathways are active in the particular environment (e.g. Booijink et al. 2010. Applied and Environmental Microbiology 76: 5533-5540). RNA so prepared can be reverse-transcribed into DNA for hybridization, amplification, and sequence analysis.

Baits can be mixed with prepared sample DNA prior to hybridization by any means known in the art. The amount of baits added to the sample DNA should be sufficient to bind fragments of a CRISPR gene or system of interest. In some embodiments, a greater amount of baits is added to the mixture compared to the amount of sample DNA. The ratio of bait to sample DNA for hybridization can be about 1:4, about 1:3, about 1:2, about 1:1.8, about 1:1.6, about 1:1.4, about 1:1.2, about 1:1, about 2:1, about 3:1, about 4:1, about 5:1, about 10:1, about 20:1, about 50:1, or about 100:1, and higher.

While hybridization conditions may vary, hybridization of such bait sequences may be carried out under stringent conditions. By “stringent conditions” or “stringent hybridization conditions” is intended conditions under which the bait will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the bait can be identified (homologous probing). Alternatively, stringency conditions can be adjusted to allow some mismatching in sequences so that lower degrees of similarity are detected (heterologous probing). In specific embodiments, the prepared sample DNA is hybridized to the baits for 16-24 hours at about 45° C., about 50° C., about 55° C., about 60° C., about 65° C., about 70° C., or about 75° C. In particular embodiments, the prepared sample DNA is hybridized to the baits at about 65° C.

Typically, stringent conditions will be those in which the salt concentration is less than about 1.5 M Na ion, typically about 0.01 to 1.0 M Na ion concentration (or other salts) at pH 7.0 to 8.3, and the temperature is at least about 30° C. for short baits (e.g., 10 to 50 nucleotides) and at least about 60° C. for long baits (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Other exemplary high-stringency conditions are those found in SureSelect^{XT} Target Enrichment System for Illumina Paired-End Sequencing Library Protocol, Version 1.6 and NimbleGen SeqCap EZ Library SR User's Guide, Version 4.3. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium. Specificity is typically the function of post-hybridization washes, the critical factors being the ionic strength and temperature of the final wash solution. For DNA-DNA hybrids, the Tm can be approximated from the equation of Meinkoth and Wahl (1984) Anal. Biochem. 138:267-284: Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched bait. Tm is reduced by about 1° C. for each 1% of mismatching; thus, Tm, hybridization, and/or wash conditions can be adjusted to hybridize to sequences of the desired identity. For example, if sequences with >90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

As used herein, a hybridization complex refers to sample DNA fragments hybridizing to a bait. Following hybridization, the labeled baits can be separated based on the presence of the detectable label, and the unbound sequences are removed under appropriate wash conditions that remove the nonspecifically bound DNA and unbound DNA, but do not substantially remove the DNA that hybridizes specifically. The hybridization complex can be captured and purified from non-binding baits and sample DNA fragments. For example, the hybridization complex can be captured by using a binding partner of the detectable label attached to the baits, wherein the binding partner is attached to a solid phase, such as a bead or a magnetic bead. The binding partner binds in a specific manner to the detectable label. For example, in those embodiments wherein the baits are biotinylated, the binding partner can be streptavidin. In such embodiments, the hybridization complex captured onto a streptavidin coated bead, for example, can be selected by magnetic bead selection. The captured sample DNA fragment can then be amplified and index tagged for multiplex sequencing. As used herein, “index tagging” refers to the addition of a known polynucleotide sequence in order to track the sequence or provide a template for PCR. Index tagging the captured sample DNA sequences can identify the DNA source in the case that multiple pools of captured and indexed DNA are sequenced together. As used herein, an “enrichment kit” or “enrichment kit for multiplex sequencing” refers to a kit designed with reagents and instructions for preparing DNA from a complex sample and hybridizing the prepared DNA with labeled baits. In certain embodiments, the enrichment kit further provides reagents and instructions for capture and purification of the hybridization complex and/or amplification of any captured fragments of the CRISPR RGN genes or systems of interest. In specific embodiments, the enrichment kit is the SureSelect^{XT} Target Enrichment System for Illumina Paired-End Sequencing Library Protocol, Version 1.6. In other specific embodiments, the enrichment kit is as described in the NimbleGen SeqCap EZ Library SR User's Guide, Version 4.3 Alternatively, the DNA from multiple complex samples can be indexed and amplified before hybridization. In such embodiments, the enrichment kit can be the SureSelect^{XT2} Target Enrichment System for Illumina Multiplexed Sequencing Protocol, Version D.0

Following hybridization, the captured target organism DNA can be sequenced by any means known in the art. Sequencing of nucleic acids isolated by the methods described herein is, in certain embodiments, carried out using massively parallel short-read sequencing systems such as those provided by Illumina®, Inc. (HiSeq 1000, HiSeq 2000, HiSeq 2500, Genome Analyzers, MiSeq systems), Applied Biosystems™ Life Technologies (ABI PRISM® Sequence detection systems, SOLiD™ System, Ion PGM™ Sequencer, Ion Proton™ Sequencer), because the read out generates more bases of sequence per sequencing unit than other sequencing methods that generate fewer but longer reads. Sequencing can also be carried out by methods generating longer reads, such as those provided by Oxford Nanopore Technologies® (GridiON, MiniON) or Pacific Biosciences (Pachio RS II), to provide a sequence read of the full length sequence of the variant of the CRISPR RGN gene or system of interest, in order to avoid assembling various shorter sequences. Sequencing can also be carried out by standard Sanger dideoxy terminator sequencing methods and devices, or on other sequencing instruments, further as those described in, for example, United States patents and U.S. Pat. Nos. 5,888,737, 6,175,002, 5,695,934, 6, 140,489, 5,863,722, 2007/007991, 2009/0247414, 2010/01 11768 and PCT application WO2007/123744 each of which is incorporated herein by reference in its entirety.

In some embodiments, sequences can be assembled by any means known in the art. The sequences of individual fragments of variants of CRISPR RGN genes or systems of interest can be assembled to identify the full length sequence of the variant of the CRISPR RGN gene or system of interest. In some embodiments, sequences are assembled using the CLC Bio suite of bioinformatics tools. Following assembly, sequences of variants of the CRISPR RGN genes or systems of interest are searched (e.g., sequence similarity search) against a database of known sequences including those of the CRISPR RGN genes or systems of interest in order to identify the variant of the CRISPR RGN gene or system of interest. In this manner, new variants (i.e., homologs) of CRISPR RGN genes and systems of interest can be identified from complex samples.

Given the low sequence identity between many CRISPR RGN genes, however, sequences of CRISPR RGN gene variants can also be analyzed for the presence of domains present in known CRISPR RGN genes, including but not limited to, RuvC domains, HNH domains, and PAM interacting domains. See, for example, Sapranauskas et al. (2011) Nucleic Acids Res 39:9275-9282 and Nishimasu et al. (2014) Cell 156(5):935-949, each of which is herein incorporated by reference in its entirety. The RuvC domain of Streptococcus pyogenes Cas9, for example, consists of a six-stranded mixed beta sheet flanked by alpha helices and two additional two-stranded antiparallel beta sheets and shares structural similarity with the retroviral integrase superfamily members characterized by an RNase H fold, such as E. coli RuvC (PDB code 1HJR) and Thermus thermophilus RuvC (PDB code 4LD0). RuvC nucleases have four catalytic residues (e.g., Asp10, Glu762, His983, and Asp986 in S. pyogenes Cas9) and cleave Holliday junctions. The HNH domain of S. pyogenes Cas9, for example, comprises a two-stranded antiparallel beta sheet flanked by four alpha helices and it shares structural similarity with the HNH endonucleases characterized by a ββα-metal fold, such as phage T4 endonuclease VII (PDB code 2QNC) and Vibrio vulnificus nuclease (PDB code 1OUP). HNH nucleases have three catalytic residues (e.g., Asp839, His 840, and Asn863 in S. pyogenes Cas9) and cleave nucleic acid substrates through a single-metal mechanism. The PAM-interacting domain of S. pyogenes Cas9 comprises residues 1099-1368, for example.

If a complete CRISPR system is desired, the flanking sequences of the variant of a CRISPR RGN gene of interest can be sequenced and analyzed to identify the tracrRNA-coding sequence, and thus, the tracrRNA sequence. One of ordinary skill in the art will appreciate that often tracrRNAs are encoded on the opposite coding strand from the RGN and often are within about 60 to about 100 nucleotides from the RGN-encoding sequence, either in the 5′ or 3′ direction. Methods for identifying the tracrRNA sequence include scanning the flanking sequences for a known antirepeat-coding sequence or a variant thereof. CRISPR repeat and antirepeat sequences utilized by known CRISPR RGNs are known in the art and can be found, for example, at the CRISPR database on the world wide web at crispr.i2bc.paris-saclay.fr/crispr/. Alternatively, a tracrRNA sequence can be identified by predicting the secondary structure of sequences encoding by the flanking sequences using any known computational method, including but not limited to NUPACK RNA folding software (Dirks et al. (2007) SIAM Review 49(1):65-88, which is incorporated herein in its entirety), and searching for secondary structures similar to those described herein and outlined in Briner et al. (2014) Molecular Cell 56:333-339, Briner and Barrangou (2016) Cold Spring Harb Protoc; doi: 10.1101/pdb.top090902, and U.S. Publication No. 2017/0275648 (each of which is incorporated herein by reference in its entirety), including but not limited to a nexus hairpin and a transcription-terminating hairpin. The CRISPR repeat sequence of the corresponding crRNA can then be deduced based on the identified anti-repeat sequence of the tracrRNA by generating a CRISPR repeat sequence that is fully or partially complementary to the anti-repeat sequence of the tracrRNA. The sequence of the remaining crRNA can be generated by incorporating functional modules seen in guide RNAs, including the lower stem, bulge, and upper stem.

In some embodiments, the method for identifying the tracrRNA-coding region and thus, the tracrRNA, comprises the development and use of Hidden Markov Models (HMMs) of RNA structures and sequences using previously published tracrRNAs (see, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety), as well as any previously identified tracrRNA sequences.

One of ordinary skill in the art will appreciate that for those CRISPR systems that are not expected to comprise a tracrRNA (e.g., Types V-A, VI), often the structure of the CRISPR repeat of the crRNA is more important than the actual sequence of the CRISPR repeat. Thus, various known crRNAs (or variants comprising similar structure) from known Type V-A or VI CRISPR RGNs can be paired with these types of CRISPR RGNs in order to obtain a complete CRISPR system. See, for example, Shmakov et al. (2015) Mol Cell 60(3):385-397, which is herein incorporated by reference in its entirety. CRISPR systems that are not expected to comprise a tracrRNA are those that are identified using baits designed from known Type V-A or Type VI CRISPR systems or those that exhibit homology with these CRISPR systems. Alternatively, the inability to identify a tracrRNA in flanking sequences based on homology with known anti-repeat sequences or known tracrRNA secondary structures might indicate that the CRISPR system does not comprise a tracrRNA.

In some embodiments, the presently disclosed methods can further comprise a step of assaying for binding between the guideRNA and the newly identified CRISPR RGN. For these assays, a single guide RNA can be constructed in which both the crRNA and tracrRNA are comprised within a single RNA molecule. Generally, a linker sequence of at least 3 nucleotides separates the crRNA and tracrRNA on single guide RNAs. One of ordinary skill in the art will understand that the linker sequence should not comprise complementary bases in order to avoid the formation of a stem loop structure within or comprising the linker sequence. Alternatively, two distinct RNA molecules comprising the crRNA and the tracrRNA, respectively, can be used for this analysis, wherein the two RNA molecules are hybridized to one another through the CRISPR repeat sequence of the crRNA and the anti-repeat portion of the tracrRNA, which is referred to herein as a dual-guide RNA. For those CRISPR RGNs that are not expected to utilize a tracrRNA, the guide RNA comprises a single crRNA molecule. The single guide RNA, dual-guide RNA, or crRNA can be synthesized chemically or via in vitro transcription.

Assays for determining sequence-specific binding between a CRISPR RGN and a guide RNA are known in the art and include, but are not limited to, in vitro binding assays between an expressed CRISPR RGN and the guideRNA, which can be tagged with a detectable label (e.g., biotin) and used in a pull-down detection assay in which the guideRNA:CRISPR RGN complex is captured via the detectable label (e.g., with streptavidin beads). A control guideRNA with an unrelated sequence or structure to the guideRNA can be used as a negative control for non-specific binding of the CRISPR RGN to RNA.

In certain embodiments, if one wishes to use the identified CRISPR system for genome editing or for targeting a genomic location, the presently disclosed methods can further comprise steps wherein the preferred protospacer adjacent motif (PAM) sequence is identified for the novel CRISPR system. A protospacer adjacent motif is generally within about 1 to about 10 nucleotides from the target nucleotide sequence, including about 1, about 2, about 3, about 4, about 5, about 6, about 7, about 8, about 9, or about 10 nucleotides from the target nucleotide sequence. The PAM can be 5′ or 3′ of the target sequence. Generally, the PAM is a consensus sequence of about 3-4 nucleotides, but in particular embodiments, can be 2, 3, 4, 5, 6, 7, 8, 9, or more nucleotides in length. Methods for identifying a preferred PAM sequence or consensus sequence for a given CRISPR RGN are known in the art and include, but are not limited to the PAM depletion assay described by Karvelis et al. (2015) Genome Biol 16:253, or the assay disclosed in Pattanayak et al. (2013) Nat Biotechnol 31(9):839-43, each of which is incorporated by reference in its entirety.

The methods can further comprise a step of assaying for the ability of the identified CRISPR RGN, in association with its guideRNA, to bind to a target sequence and/or to cleave the target sequence in a sequence-specific manner. Methods to measure binding of a CRISPR RGN to a target sequence are known in the art and include chromatin immunoprecipitation assays, gel mobility shift assays, DNA pull-down assays, reporter assays, microplate capture and detection assays. Likewise, methods to measure cleavage or modification of a target sequence are known in the art and include in vitro or in vivo cleavage assays wherein cleavage is confirmed using PCR, sequencing, or gel electrophoresis, with or without the attachment of an appropriate label (e.g., radioisotope, fluorescent substance) to the target sequence to facilitate detection of degradation products. Alternatively, the nicking triggered exponential amplification reaction (NTEXPAR) assay can be used (see, e.g., Zhang et al. (2016) Chem. Sci. 7:4951-4957). In vivo cleavage can be evaluated using the Surveyor assay (Guschin et al. (2010) Methods Mol Biol 649:247-256).

In order to assay for the ability of the identified CRISPR RGN to bind to the guide RNA or to a target sequence and/or to cleave the target sequence in a sequence-specific manner, a polynucleotide encoding the identified CRISPR RGN can be expressed in an in vitro system or cellular system and can be purified using any method known in the art.

An “isolated” or “purified” polynucleotide or polypeptide, or biologically active portion thereof, is substantially or essentially free from components that normally accompany or interact with the polynucleotide or polypeptide as found in its naturally occurring environment. Thus, an isolated or purified polynucleotide or polypeptide is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

The purified CRISPR RGN can be combined with its guide RNA in such a manner to allow for the formation of a ribonucleoprotein complex. Alternatively, a ribonucleoprotein complex comprising the identified CRISPR RGN can be purified from a cell or organism that has been transformed with polynucleotides that encode the RGN and a guide RNA and cultured under conditions that allow for the expression of the RGN polypeptide and guide RNA. The ribonucleoprotein complex can then be purified from a lysate of the cultured cells.

Methods for purifying an RGN polypeptide or RGN ribonucleoprotein complex from a lysate of a biological sample are known in the art (e.g., size exclusion and/or affinity chromatography, 2D-PAGE, HPLC, reversed-phase chromatography, immunoprecipitation). To enable purification, the identified CRISPR RGN can be fused to a purification tag (e.g., glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, E2, FLAG, HA, nus, Softag 1, Softag 3, Strep, SBP, Glu-Glu, HSV, KT3, S, 51, T7, V5, VSV-G, 6×His, biotin carboxyl carrier protein (BCCP), and calmodulin).

### IV. Kits for Identification of a Variant of a CRISPR RGN Gene or System of Interest

Kits are provided for identifying variants of CRISPR RGN genes or systems of interest by the methods disclosed herein. The kits include a bait pool or RNA bait pool, or reagents suitable for producing a bait pool specific for a CRISPR RGN gene or system of interest, along with other reagents, such as a solid phase containing a binding partner of any detectable label on the baits. In specific embodiments, the detectable label is biotin and the binding partner streptavidin or streptavidin adhered to magnetic beads. The kits may also include solutions for hybridization, washing, or eluting of the DNA/solid phase compositions described herein, or may include a concentrate of such solutions.

TABLE 1
Exemplary CRISPR RGN genes of interest.

| | | NCBI | | | | |
|---|---|---|---|---|---|---|
| Name | Acc. No. | Protein | NCBI Nuc | Authors | Year | Source Strain |
| Cas12a-1 | AWUR01000016.1 | | HMPREF1246_0236 | Shmakov et al | 2017 | Acidaminococcus_BV3L6_ |
| | | | | | | BV3L6 |
| Cas12a-2 | KK211384.1 | | KK211384.1_16 | Shmakov et al | 2017 | Anaerovibrio_RM50_ |
| | | | | | | RM50 |
| Cas12a-3 | JAIQ01000039.1 | | AA20_01655 | Shmakov et al | 2017 | Arcobacter_butzleri_L348 |
| Cas12a-4 | KQ959253.1 | | HMPREF1869_00137 | Shmakov et al | 2017 | Bacteroidales_bacterium_ |
| | | | | | | KA00251_KA00251 |
| Cas12a-5 | GG774890.1 | | HMPREF0156_01430 | Shmakov et al | 2017 | Bacteroidetes_oral_taxon_ |
| | | | | | | 274_F0058 |
| Cas12a-6 | AUKC01000013.1 | | AUKC01000013.1_3 | Shmakov et al | 2017 | Butyrivibrio_NC3005_NC3005 |
| Cas12a-7 | AUKD01000009.1 | | AUKD01000009.1_66 | Shmakov et al | 2017 | Butyrivibrio_fibrisolvens_MD2001 |
| Cas12a-8 | CP001812.1 | | bpr_II405 | Shmakov et al | 2017 | Butyrivibrio_proteoclasticus_B316 |
| Cas12a-9 | LCAP01000004.1 | | UU43_C0004G0003 | Shmakov et al | 2017 | Candidatus_Falkowbacteria_ |
| | | | | | | bacterium_GW2011_GWA2_41_14 |
| Cas12a-10 | CP004049.1 | | MMALV_08950 | Shmakov et al | 2017 | Candidatus_Methanomethylophilus_ |
| | | | | | | alvus_Mx1201 |
| Cas12a-11 | CP010070.1 | | Mpt1_c09950 | Shmakov et al | 2017 | Candidatus_Methanoplasma_ |
| | | | | | | termitum_MpT1 |
| Cas12a-12 | LBOO01000015.1 | | UR27_C0015G0004 | Shmakov et al | 2017 | Candidatus_Peregrinibacteria_ |
| | | | | | | bacterium_GW2011_GWA2_33_10 |
| Cas12a-13 | LBOR01000010.1 | | UR30_C0010G0003 | Shmakov et al | 2017 | Candidatus_Peregrinibacteria_ |
| | | | | | | bacterium_GW2011_GWC2_33_13 |
| Cas12a-14 | LBTJ01000016.1 | | US54_C00_16G0015 | Shmakov et al | 2017 | Candidatus_Roizmanbacteria_ |
| | | | | | | bacterium_GW2011_GWA2_37_7 |
| Cas12a-15 | FR903162.1 | | BN720_00865 | Shmakov et al | 2017 | Eubacterium_CAG_581 |
| Cas12a-16 | FR902996.1 | | BN774_00378 | Shmakov et al | 2017 | Eubacterium_CAG_76 |
| Cas12a-17 | CP001104.1 | | EUBELI_01419 | Shmakov et al | 2017 | Eubacterium_eligens_ |
| | | | | | | ATCC_27750 |
| Cas12a-18 | FR878942.1 | | BN765_00730 | Shmakov et al | 2017 | Eubacterium_eligens_CAG_72 |
| Cas12a-19 | JYGZ01000006.1 | | SY27_14115 | Shmakov et al | 2017 | Flavobacterium_316_316 |
| Cas12a-20 | FQ859183.1 | | FBFL15_2587 | Shmakov et al | 2017 | Flavobacterium_branchiophilum_ |
| | | | | | | FL_15 |
| Cas12a-21 | CP002557.1 | | FNFX1_1431 | Shmakov et al | 2017 | Francisella_cf_novicida_Fx1 |
| Cas12a-22 | CP009444.1 | | LA02_1347 | Shmakov et al | 2017 | Francisella_philomiragia_ |
| | | | | | | GA01_2801 |
| Cas12a-23 | CP009353.1 | | AS84_1114 | Shmakov et al | 2017 | Francisella_tularensis_ |
| | | | | | | novicida_F6168 |
| Cas12a-24 | DS989819.1 | | FTE_0784 | Shmakov et al | 2017 | Francisella_tularensis_ |
| | | | | | | novicida_FTE |
| Cas12a-25 | DS995364.1 | | FTG_0873 | Shmakov et al | 2017 | Francisella_tularensis_ |
| | | | | | | novicida_FTG |
| Cas12a-26 | DS264129.1 | | FTCG_00909 | Shmakov et al | 2017 | Francisella_tularensis_novicida_ |
| | | | | | | GA99_3549 |
| Cas12a-27 | KN046811.1 | | DR83_652 | Shmakov et al | 2017 | Francisella_tularensis_novicida |
| Cas12a-28 | CP000439.1 | | FTN_1397 | Shmakov et al | 2017 | Francisella_tularensis_novicida_U112 |
| Cas12a-29 | CP009633.1 | | AW25_605 | Shmakov et al | 2017 | Francisella_tularensis_novicida_U112 |
| Cas12a-30 | CP010103.1 | | CH70_544 | Shmakov et al | 2017 | Francisella_tularensis_tularensis |
| Cas12a-31 | LFLB01000034.1 | | LFLB01000034.1_7 | Shmakov et al | 2017 | Gammaproteobacteria_bacterium_ |
| | | | | | | LS_SOB |
| Cas12a-32 | JH601088.1 | | HMPREF9709_01099 | Shmakov et al | 2017 | Helcococcus_kunzii_ATCC_ |
| | | | | | | 51366 |
| Cas12a-33 | KE159629.1 | | C809_02517 | Shmakov et al | 2017 | Lachnospiraceae_bacterium_ |
| | | | | | | COE1_COE1 |
| Cas12a-34 | JQKK01000008.1 | | JQKK01000008.1_137 | Shmakov et al | 2017 | Lachnospiraceae_bacterium_ |
| | | | | | | MA2020_MA2020 |
| Cas12a-35 | KL370807.1 | | KL370807.1_38 | Shmakov et al | 2017 | Lachnospiraceae_bacterium_ |
| | | | | | | MC2017_MC2017 |
| Cas12a-36 | KL370807.1 | | KL370807.1_39 | Shmakov et al | 2017 | Lachnospiraceae_bacterium_ |
| | | | | | | MC2017_MC2017 |
| Cas12a-37 | JHWS01000001.1 | | JHWS01000001.1_302 | Shmakov et al | 2017 | Lachnospiraceae_bacterium_ |
| | | | | | | NC2008_NC2008 |
| Cas12a-38 | JNKS01000011.1 | | JNKS01000011.1_50 | Shmakov et al | 2017 | Lachnospiraceae_bacterium_ |
| | | | | | | ND2006_ND2006 |
| Cas12a-39 | AHMM02000017.1 | | LEP1GSC047_3100 | Shmakov et al | 2017 | Leptospira_inadai_Lyme_10 |
| Cas12a-40 | AOMT01000011.1 | | MBO_03467 | Shmakov et al | 2017 | Moraxella_bovoculi_237 |
| Cas12a-41 | KE384587.1 | | KE384587.1_6 | Shmakov et al | 2017 | Moraxella_caprae_DSM_19149 |
| Cas12a-42 | KE384190.1 | | KE384190.1_9 | Shmakov et al | 2017 | Oribacterium_NK2B42_ |
| | | | | | | NK2B42 |
| Cas12a-43 | LCIC01000001.1 | | UW39_C0001G0044 | Shmakov et al | 2017 | Parcubacteria_group_bacterium_ |
| | | | | | | GW2011_GWC2_44_17 |
| Cas12a-44 | LCID01000007.1 | | UW40_C0007G0006 | Shmakov et al | 2017 | Parcubacteria_group_bacterium_ |
| | | | | | | GW2011_GWF2_44_17 |
| Cas12a-45 | JQJC01000021.1 | | HQ38_07045 | Shmakov et al | 2017 | Porphyromonas_crevioricanis_ |
| | | | | | | COT_253_OH1447 |
| Cas12a-46 | JQJB01000003.1 | | HQ45_01350 | Shmakov et al | 2017 | Porphyromonas_crevioricanis_ |
| | | | | | | COT_253_OH2125 |
| Cas12a-47 | BAOV01000052.1 | | PORCAN_2094 | Shmakov et al | 2017 | Porphyromonas_crevioricanis_ |
| | | | | | | JCM_13913 |
| Cas12a-48 | BAOU01000008.1 | | PORCRE_269 | Shmakov et al | 2017 | Porphyromonas_crevioricanis_ |
| | | | | | | JCM_15906 |
| Cas12a-49 | JRFB01000011.1 | | HR11_04570 | Shmakov et al | 2017 | Porphyromonas_macacae_ |
| | | | | | | COT_192_OH2631 |
| Cas12a-50 | KB904124.1 | | KB904124.1_428 | Shmakov et al | 2017 | Porphyromonas_macacae_ |
| | | | | | | DSM_20710_JCM_13914 |
| Cas12a-51 | BAKQ01000001.1 | | BAKQ01000001.1_129 | Shmakov et al | 2017 | Porphyromonas_macacae_ |
| | | | | | | DSM_20710_JCM_13914 |
| Cas12a-52 | AUFP01000002.1 | | AUFP01000002.1_257 | Shmakov et al | 2017 | Prevotella_albensis_DSM_ |
| | | | | | | 11370_JCM_12258 |
| Cas12a-53 | BAJD01000001.1 | | BAJD01000001.1_53 | Shmakov et al | 2017 | Prevotella_albensis_ |
| | | | | | | DSM_11370_JCM_12258 |
| Cas12a-54 | KK211334.1 | | KK211334.1_60 | Shmakov et al | 2017 | Prevotella_brevis_ATCC_19188 |
| Cas12a-55 | ADWO01000096.1 | | PBR_0786 | Shmakov et al | 2017 | Prevotella_bryantii_B14 |
| Cas12a-56 | JRNR01000108.1 | | HMPREF0654_09810 | Shmakov et al | 2017 | Prevotella_disiens_ |
| | | | | | | DNF00882 |
| Cas12a-57 | AEDO01000031.1 | | HMPREF9296_0755 | Shmakov et al | 2017 | Prevotella_disiens_FB035_09AN |
| Cas12a-58 | KE384028.1 | | KE384028.1_43 | Shmakov et al | 2017 | Proteocatella_sphenisci_DSM_23131 |
| Cas12a-59 | KE384121.1 | | KE384121.1_68 | Shmakov et al | 2017 | Pseudobutyrivibrio_ruminis_CF1b |
| Cas12a-60 | JQDQ01000121.1 | | ER57_07115 | Shmakov et al | 2017 | Smithella_SCADC |
| Cas12a-61 | JMED01000006.1 | | DS62_13820-2 | Shmakov et al | 2017 | Smithella_SC_K08D17 |
| Cas12a-62 | CP011280.1 | | VC03_02970 | Shmakov et al | 2017 | Sneathia_amnii_SN35 |
| Cas12a-63 | KL370853.1 | | KL370853.1_80 | Shmakov et al | 2017 | Succinivibrio_dextrinosolvens_H5 |
| Cas12a-64 | GL995220.1 | | GL995220.1_19 | Shmakov et al | 2017 | Succinivibrionaceae_bacterium_ |
| | | | | | | WG_1_WG_1 |
| Cas12a-65 | JMKI01000031.1 | | EH55_04135 | Shmakov et al | 2017 | Synergistes_jonesii_78_1 |
| Cas12a-66 | LQBO01000001.1 | | AVO42_04040 | Shmakov et al | 2017 | Thiomicrospira_XS5_XS5 |
| Cas12a-67 | BBPX01000040.1 | | BBPX01000040.1_1 | Shmakov et al | 2017 | Treponema_endosymbiont_of_ |
| | | | | | | Eucomonympha_D2 |
| Cas12a-68 | BBPY01000028.1 | | BBPY01000028.1_15 | Shmakov et al | 2017 | Treponema_endosymbiont_of_ |
| | | | | | | Eucomonympha_E12 |
| Cas12a-69 | BBPZ01000036.1 | | BBPZ01000036.1_1 | Shmakov et al | 2017 | Treponema_endosymbiont_of_ |
| | | | | | | Eucomonympha_E8 |
| Cas12a-70 | LBTH01000007.1 | | US52_C0007G0008 | Shmakov et al | 2017 | candidate_division_WS6_ |
| | | | | | | bacterium_GW2011_GWA2_37_6 |
| Cas12a-71 | ADJS01008976 | | ADJS01008976_1 | Shmakov et al | 2017 | uncultured |
| Cas12b-1 | BCQI01000053.1 | | BCQI01000053.1_4 | Shmakov et al | 2017 | Alicyclobacillus_acidiphilus_ |
| | | | | | | NBRC_100859 |
| Cas12b-2 | AURB01000127.1 | | N007_06525 | Shmakov et al | 2017 | Alicyclobacillus_acidoterrestris_ |
| | | | | | | ATCC_49025 |
| Cas12b-3 | KE386913.1 | | KE386913.1_1 | Shmakov et al | 2017 | Alicyclobacillus_contaminans_ |
| | | | | | | DSM_17975 |
| Cas12b-4 | BCRP01000027.1 | | BCRP01000027.1_17 | Shmakov et al | 2017 | Alicyclobacillus_kakegawensis_ |
| | | | | | | NBRC_103104 |
| Cas12b-5 | BCQV01000052.1 | | BCQV01000052.1_10 | Shmakov et al | 2017 | Alicyclobacillus_shizuokensis_ |
| | | | | | | NBRC_103103 |
| Cas12b-6 | KI301973.1 | | KI301973.1_306 | Shmakov et al | 2017 | Bacillus_NSP2_1 |
| Cas12b-7 | JXLT01000152.1 | | B4166_3744 | Shmakov et al | 2017 | Bacillus_thermoamylovorans_ |
| | | | | | | B4166 |
| Cas12b-8 | JXLU01000068.1 | | B4167_2499 | Shmakov et al | 2017 | Bacillus_thermoamylovorans_ |
| | | | | | | B4167 |
| Cas12b-9 | AKKB01000053.1 | | PMI08_01933 | Shmakov et al | 2017 | Brevibacillus_CF112_CF112 |
| Cas12b-10 | AOBR01000150.1 | | D478_25088 | Shmakov et al | 2017 | Brevibacillus_agri_BAB_2500 |
| Cas12b-11 | AOBR01000150.1 | | D478_25093 | Shmakov et al | 2017 | Brevibacillus_agri_BAB_2500 |
| Cas12b-12 | LMXM01000006.1 | | LMXM01000006.1_115 | Shmakov et al | 2017 | Chloracidobacterium_ |
| | | | | | | thermophilum_OC1 |
| Cas12b-13 | KE386988.1 | | KE386988.1_31 | Shmakov et al | 2017 | Desulfatirhabdium_butyrativorans_ |
| | | | | | | DSM_18734 |
| Cas12b-14 | JPIK01000006.1 | | JPIK01000006.1_72 | Shmakov et al | 2017 | Desulfonatronum_thiodismutans_ |
| | | | | | | MLF_1 |
| Cas12b-15 | KE386879.1 | | KE386879.1_222 | Shmakov et al | 2017 | Desulfovibrio_inopinatus_ |
| | | | | | | DSM_10711 |
| Cas12b-16 | CP001349.1 | | Mnod_0560 | Shmakov et al | 2017 | Methylobacterium_nodulans_ |
| | | | | | | ORS_2060 |
| Cas12b-17 | CP001349.1 | | Mnod_0561 | Shmakov et al | 2017 | Methylobacterium_nodulans_ |
| | | | | | | ORS_2060 |
| Cas12b-18 | CP007053.1 | | OPIT5_03625 | Shmakov et al | 2017 | Opitutaceae_bacterium_ |
| | | | | | | TAV5_TAV5 |
| Cas12b-19 | LNAA01000001.1 | | LNAA01000001.1_1060 | Shmakov et al | 2017 | Oscillatoriales_cyanobacterium_ |
| | | | | | | MTP1_MTP1 |
| Cas12b-20 | KE387196.1 | | KE387196.1_31 | Shmakov et al | 2017 | Tuberibacillus_calidus_ |
| | | | | | | DSM_17572 |
| Cas13a-1 | CVRQ01000008.1 | | T1815_05231 | Shmakov et al | 2017 | Agathobacter_rectalis_T1_815 |
| Cas13a-2 | JQLU01000005.1 | | JQLU01000005.1_155 | Shmakov et al | 2017 | Carnobacterium_gallinarum_ |
| | | | | | | DSM_4847 |
| Cas13a-3 | JQLU0000005.1 | | JQLU01000005.1_2303 | Shmakov et al | 2017 | Carnobacterium_gallinarum_ |
| | | | | | | DSM_4847 |
| Cas13a-4 | JONJ01000012.1 | | JONJ01000012.1_8 | Shmakov et al | 2017 | Clostridium_aminophilum_ |
| | | | | | | DSM_10710 |
| Cas13a-5 | DS499551.1 | | EUBSIR_02687 | Shmakov et al | 2017 | Eubacterium_siraeum_DSM_15702 |
| Cas13a-6 | KB907524.1 | | KB907524.1_67 | Shmakov et al | 2017 | Eubacterium_siraeum_DSM_15702 |
| Cas13a-7 | CVTD020000026 | | CVTD020000026_43 | Shmakov et al | 2017 | Herbinix |
| Cas13a-8 | JQKK01000015.1 | | JQKK01000015.1_80 | Shmakov et al | 2017 | Lachnospiraceae_bacterium_ |
| | | | | | | MA2020_MA2020 |
| Cas13a-9 | AUJT01000030.1 | | AUJT01000030.1_16 | Shmakov et al | 2017 | Lachnospiraceae_bacterium_ |
| | | | | | | NK4A144_NK4A144 |
| Cas13a-10 | ATWC01000054.1 | | ATWC01000054.1_6 | Shmakov et al | 2017 | Lachnospiraceae_bacterium_ |
| | | | | | | NK4A179_NK4A179 |
| Cas13a-11 | CP001685.1 | | Lebu_1799 | Shmakov et al | 2017 | Leptotrichia_buccalis_C_1013_b |
| Cas13a-12 | KI272904.1 | | HMPREF9108_01633 | Shmakov et al | 2017 | Leptotrichia_oral_taxon_ |
| | | | | | | 225_F0581 |
| Cas13a-13 | KI271320.1 | | HMPREF1552_00123 | Shmakov et al | 2017 | Leptotrichia_oral_taxon_ |
| | | | | | | 879_F0557 |
| Cas13a-14 | KB890278.1 | | KB890278.1_32 | Shmakov et al | 2017 | Leptotrichia_shahii_DSM_19757 |
| Cas13a-15 | KI271395.1 | | HMPREF9015_00520 | Shmakov et al | 2017 | Leptotrichia_wadei_F0279 |
| Cas13a-16 | KI271421.1 | | HMPREF9015_01858 | Shmakov et al | 2017 | Leptotrichia_wadei_F0279 |
| Cas13a-17 | KI271424.1 | | HMPREF9015_02301 | Shmakov et al | 2017 | Leptotrichia_wadei_F0279 |
| Cas13a-18 | JNFB01000012.1 | | EP58_05535 | Shmakov et al | 2017 | Listeria_newyorkensis_FSL_ |
| | | | | | | M6_0635 |
| Cas13a-19 | FN557490.1 | | lse_1149 | Shmakov et al | 2017 | Listeria_seeligeri_1_2b_ |
| | | | | | | SLCC3954 |
| Cas13a-20 | AODJ01000004.1 | | PWEIH_02614 | Shmakov et al | 2017 | Listeria_weihenstephanensis_ |
| | | | | | | FSL_R9_0317 |
| Cas13a-21 | CP002345.1 | | Palpr_0179 | Shmakov et al | 2017 | Paludibacter_propionicigenes_WB4 |
| Cas13a-22 | AYPR01000020.1 | | U714_11360 | Shmakov et al | 2017 | Rhodobacter_capsulatus_DE442 |
| Cas13a-23 | AYQC01000019.1 | | U717_11515 | Shmakov et al | 2017 | Rhodobacter_capsulatus_R121 |
| Cas13a-24 | CP001312.1 | | RCAP_rcc02005 | Shmakov et al | 2017 | Rhodobacter_capsulatus_SB_1003 |
| Cas13a-25 | AYQB01000025.1 | | U715_11520 | Shmakov et al | 2017 | Rhodobacter_capsulatus_Y262 |
| Cas13a-26 | FR890758.1 | | BN714_01570 | Shmakov et al | 2017 | Ruminococcus_CAG_57 |
| Cas13a-27 | LARF01000048.1 | | LARF01000048.1_8 | Shmakov et al | 2017 | Ruminococcus_N15_MGS_57 |
| Cas13a-28 | HF545617.1 | | RBI_II00459 | Shmakov et al | 2017 | Ruminococcus_bicirculans_80_3 |
| Cas13a-29 | ACOK01000100.1 | | ACOK01000100.1_5 | Shmakov et al | 2017 | Ruminococcus_flavefaciens_FD_1 |
| Cas13a-30 | ADJS01008410 | | ADJS01008410_2 | Shmakov et al | 2017 | uncultured |
| Cas13b-1 | JTLD01000029.1 | | JTLD01000029.1_31 | Shmakov et al | 2017 | Alistipes_Z0R0009_ZOR0009 |
| Cas13b-2 | CM001167.1 | | Bcop_1349-2 | Shmakov et al | 2017 | Bacteroides_coprosuis_DSM_18011 |
| Cas13b-3 | KE993153.1 | | HMPREF1981_03090 | Shmakov et al | 2017 | Bacteroides_pyogenes_F0041 |
| Cas13b-4 | BAIU01000001.1 | | JCM10003_349 | Shmakov et al | 2017 | Bacteroides_pyogenes_JCM_10003 |
| Cas13b-5 | JH932293.1 | | HMPREF9699_02005 | Shmakov et al | 2017 | Bergeyella_zoohelcum_ATCC_43767 |
| Cas13b-6 | CDOK01000028.1 | | CCAN11_1230002 | Shmakov et al | 2017 | Capnocytophaga_canimorsus_Cc11 |
| Cas13b-7 | CP002113.1 | | Ccan_11650 | Shmakov et al | 2017 | Capnocytophaga_canimorsus_Cc5 |
| Cas13b-8 | CDOD01000002.1 | | CCYN2B_100060 | Shmakov et al | 2017 | Capnocytophaga_cynodegmi_Ccyn2B |
| Cas13b-9 | KN549099.1 | | KN549099.1_981 | Shmakov et al | 2017 | Chryseobacterium_YR477_YR477 |
| Cas13b-10 | JYGZ01000003.1 | | SY27_06350 | Shmakov et al | 2017 | Flavobacterium_316_316 |
| Cas13b-11 | FQ859183.1 | | FBFL15_2182 | Shmakov et al | 2017 | Flavobacterium_branchiophilum_ |
| | | | | | | FL_15 |
| Cas13b-12 | CP013992.1 | | AWN65_03295 | Shmakov et al | 2017 | Flavobacterium_columnare_94_081 |
| Cas13b-13 | CP003222.2 | | FCOL_07235 | Shmakov et al | 2017 | Flavobacterium_columnare_ |
| | | | | | | ATCC_49512 |
| Cas13b-14 | KE161016.1 | | HMPREF9712_03108 | Shmakov et al | 2017 | Myroides_odoratimimus_ |
| | | | | | | CCUG_10230 |
| Cas13b-15 | JH590834.1 | | HMPREF9714_02132 | Shmakov et al | 2017 | Myroides_odoratimimus_ |
| | | | | | | CCUG_12901 |
| Cas13b-16 | JH815535.1 | | HMPREF9711_00870 | Shmakov et al | 2017 | Myroides_odoratimimus_ |
| | | | | | | CCUG_3837 |
| Cas13b-17 | CP013690.1 | | AS202_188_15 | Shmakov et al | 2017 | Myroides_odoratimimus_ |
| | | | | | | PR63039 |
| Cas13b-18 | CP002345.1 | | Palpr_2606 | Shmakov et al | 2017 | Paludibacter_propionicigenes_WB4 |
| Cas13b-19 | JPOS010000l8.1 | | IX84_07840 | Shmakov et al | 2017 | Phaeodactylibacter_ |
| | | | | | | xiamenensis_KD52 |
| Cas13b-20 | JQZY01000014.1 | | HQ50_05870 | Shmakov et al | 2017 | Porphyromonas_COT_052_ |
| | | | | | | OH4946_COT_052_OH4946 |
| Cas13b-21 | CP012889.1 | | PGF_00012420 | Shmakov et al | 2017 | Porphyromonas_gingivalis_381 |
| Cas13b-22 | CP012889.1 | | PGF_00016090 | Shmakov et al | 2017 | Porphyromonas_gingivalis_381 |
| Cas13b-23 | CP011995.1 | | PGA7_00008170 | Shmakov et al | 2017 | Porphyromonas_gingivalis_A7436 |
| Cas13b-24 | CP011995.1 | | PGA7_00015700 | Shmakov et al | 2017 | Porphyromonas_gingivalis_A7436 |
| Cas13b-25 | CP013131.1 | | PGS_00015470 | Shmakov et al | 2017 | Porphyromonas_gingivalis_ |
| | | | | | | A7A1_28 |
| Cas13b-26 | CP011996.1 | | PGJ_00015140 | Shmakov et al | 2017 | Porphyromonas_gingivalis_ |
| | | | | | | AJW4 |
| Cas13b-27 | AP009380.1 | | PGN_1263 | Shmakov et al | 2017 | Porphyromonas_gingivalis_ |
| | | | | | | ATCC_33277 |
| Cas13b-28 | AP009380.1 | | PGN_1623 | Shmakov et al | 2017 | Porphyromonas_gingivalis_ |
| | | | | | | ATCC_33277 |
| Cas13b-29 | BCBV01000109.1 | | PGANDO_1674 | Shmakov et al | 2017 | Porphyromonas_gingivalis_ |
| | | | | | | Ando |
| Cas13b-30 | KI259867.1 | | HMPREF1988_02131 | Shmakov et al | 2017 | Porphyromonas_gingivalis_ |
| | | | | | | F0185 |
| Cas13b-31 | KI259960.1 | | HMPREF1988_01768 | Shmakov et al | 2017 | Porphyromonas_gingivalis_ |
| | | | | | | F0185 |
| Cas13b-32 | KI260014.1 | | HMPREF1989_02374 | Shmakov et al | 2017 | Porphyromonas_gingivalis_ |
| | | | | | | F0566 |
| Cas13b-33 | KI258974.1 | | HMPREF1553_01900 | Shmakov et al | 2017 | Porphyromonas_gingivalis_ |
| | | | | | | F0568 |
| Cas13b-34 | KI258981.1 | | HMPREF1553_02065 | Shmakov et al | 2017 | Porphyromonas_gingivalis_ |
| | | | | | | F0568 |
| Cas13b-35 | KI259080.1 | | HMPREF1554_01647 | Shmakov et al | 2017 | Porphyromonas_gingivalis_F0569 |
| Cas13b-36 | KI259168.1 | | HMPREF1555_01119 | Shmakov et al | 2017 | Porphyromonas_gingivalis_F0570 |
| Cas13b-37 | KI259218.1 | | HMPREF1555_01956 | Shmakov et al | 2017 | Porphyromonas_gingivalis_F0570 |
| Cas13b-38 | CP007756.1 | | EG14_06045 | Shmakov et al | 2017 | Porphyromonas_gingivalis_HG66 |
| Cas13b-39 | CP007756.1 | | EG14_10345 | Shmakov et al | 2017 | Porphyromonas_gingivalis_HG66 |
| Cas13b-40 | CM001843.1 | | A343_1752 | Shmakov et al | 2017 | Porphyromonas_gingivalis_ |
| | | | | | | JCVI_SC001 |
| Cas13b-41 | LOEL01000001.1 | | AT291_00385 | Shmakov et al | 2017 | Porphyromonas_gingivalis_MP4_504 |
| Cas13b-42 | LOEL01000010.1 | | AT291_05730 | Shmakov et al | 2017 | Porphyromonas_gingivalis_MP4504 |
| Cas13b-43 | KI629875.1 | | SJDPG2_03560 | Shmakov et al | 2017 | Porphyromonas_gingivalis_SJD2 |
| Cas13b-44 | AP012203.1 | | PGTDC60_1457 | Shmakov et al | 2017 | Porphyromonas_gingivalis_TDC60 |
| Cas13b-45 | KI260229.1 | | HMPREF1990_01280 | Shmakov et al | 2017 | Porphyromonas_gingivalis_W4087 |
| Cas13b-46 | KI260263.1 | | HMPREF1990_01800 | Shmakov et al | 2017 | Porphyromonas_gingivalis_W4087 |
| Cas13b-47 | AJZS01000011.1 | | HMPREF1322_1926 | Shmakov et al | 2017 | Porphyromonas_gingivalis_W50 |
| Cas13b-48 | AJZS01000051.1 | | HMPREF1322_2050 | Shmakov et al | 2017 | Porphyromonas_gingivalis_W50 |
| Cas13b-49 | AE015924.1 | | PG_0338 | Shmakov et al | 2017 | Porphyromonas_gingivalis_W83 |
| Cas13b-50 | AE015924.1 | | PG_1164 | Shmakov et al | 2017 | Porphyromonas_gingivalis_W83 |
| Cas13b-51 | KN294104.1 | | HQ42_01095 | Shmakov et al | 2017 | Porphyromonas_gulae_ |
| | | | | | | COT_052_OH1355 |
| Cas13b-52 | JRAI01000002.1 | | HR08_00310 | Shmakov et al | 2017 | Porphyromonas_gulae_ |
| | | | | | | COT_052_OH1451 |
| Cas13b-53 | JRAJ01000010.1 | | HR09_05855 | Shmakov et al | 2017 | Porphyromonas_gulae_ |
| | | | | | | COT_052_OH2179 |
| Cas13b-54 | KQ040500.1 | | HR10_10685 | Shmakov et al | 2017 | Porphyromonas_gulae_ |
| | | | | | | COT_052_OH2199 |
| Cas13b-55 | JRFD01000046.1 | | HQ46_09365 | Shmakov et al | 2017 | Porphyromonas_gulae_COT_ |
| | | | | | | 052_OH2857 |
| Cas13b-56 | JRAK01000129.1 | | HR15_09830 | Shmakov et al | 2017 | Porphyromonas_gulae_COT_ |
| | | | | | | 052_OH3439 |
| Cas13b-57 | JRAQ01000019.1 | | HQ40_043025 | Shmakov et al | 2017 | Porphyromonas_gulae_COT_ |
| | | | | | | 052_OH3471 |
| Cas13b-58 | KN300347.1 | | HR16_00525 | Shmakov et al | 2017 | Porphyromonas_gulae_COT_ |
| | | | | | | 052_OH3498 |
| Cas13b-59 | JRAT01000012.1 | | HQ49_06245 | Shmakov et al | 2017 | Porphyromonas_gulae_COT_ |
| | | | | | | 052_OH3856 |
| Cas13b-60 | JRAL01000022.1 | | HR17_04485 | Shmakov et al | 2017 | Porphyromonas_gulae_COT_ |
| | | | | | | 052_OH4119 |
| Cas13b-61 | KB899147.1 | | KB899147.1_62 | Shmakov et al | 2017 | Porphyromonas_gulae_ |
| | | | | | | DSM_15663 |
| Cas13b-62 | JHUW01000010.1 | | JHUW01000010.1_60 | Shmakov et al | 2017 | Prevotella_MA2016_ |
| | | | | | | MA2016 |
| Cas13b-63 | ALJQ01000043.1 | | HMPREF1146_2324 | Shmakov et al | 2017 | Prevotella_MSX73_MSX73 |
| Cas13b-64 | JXQI01000021.1 | | ST42_02830 | Shmakov et al | 2017 | Prevotella_P4_76_P4_76 |
| Cas13b-65 | JXQK01000043.1 | | ST44_03600 | Shmakov et al | 2017 | Prevotella_P5_119_P5_119 |
| Cas13b-66 | JXQL01000055.1 | | ST45_06380 | Shmakov et al | 2017 | Prevotella_P5_125_P5_125 |
| Cas13b-67 | JXQJ01000080.1 | | ST43_06385 | Shmakov et al | 2017 | Prevotella_P5_60_P5_60 |
| Cas13b-68 | BAKF01000019.1 | | BAKF01000019.1_53 | Shmakov et al | 2017 | Prevotella_aurantiaca_JCM_15754 |
| Cas13b-69 | GL586311.1 | | HMPREF6485_0083 | Shmakov et al | 2017 | Prevotella_buccae_ATCC_33574 |
| Cas13b-70 | GG739967.1 | | HMPREF0649_02461 | Shmakov et al | 2017 | Prevotella_buccae_D17 |
| Cas13b-71 | JVYX01000689.1 | | JVYX01000689.1_4 | Shmakov et al | 2017 | Prevotella_denticola_1205_PDEN |
| Cas13b-72 | JVYX01000736.1 | | JVYX01000736.1_6 | Shmakov et al | 2017 | Prevotella_denticola_1205_PDEN |
| Cas13b-73 | JVYU01002440.1 | | JVYU01002440.1_2 | Shmakov et al | 2017 | Prevotella_denticola_1208_PDEN |
| Cas13b-74 | BAJY01000004.1 | | BAJY01000004.1_86 | Shmakov et al | 2017 | Prevotella_falsenii_DSM_ |
| | | | | | | 22864_JCM_15124 |
| Cas13b-75 | AP014926.1 | | PI172_2270 | Shmakov et al | 2017 | Prevotella_intermedia_17_2 |
| Cas13b-76 | CP003502.1 | | PIN17_0200 | Shmakov et al | 2017 | Prevotella_intermedia_17 |
| Cas13b-77 | KE392225.1 | | KE392225.1_46 | Shmakov et al | 2017 | Prevotella_intermedia_ATCC_ |
| | | | | | | 25611_DSM_20706 |
| Cas13b-78 | JAEZ01000017.1 | | JAEZ01000017.1_46 | Shmakov et al | 2017 | Prevotella_intermedia_ATCC_ |
| | | | | | | 25611_DSM_20706 |
| Cas13b-79 | ATMK01000017.1 | | M573_117042 | Shmakov et al | 2017 | Prevotella_intermedia_ZT |
| Cas13b-80 | GL982513.1 | | HMPREF9144_1146 | Shmakov et al | 2017 | Prevotella_pallens_ATCC_700821 |
| Cas13b-81 | AWET01000045.1 | | HMPREF1218_0639 | Shmakov et al | 2017 | Prevotella_pleuritidis_F0068 |
| Cas13b-82 | BAJN01000005.1 | | BAJN01000005.1_116 | Shmakov et al | 2017 | Prevotella_pleuritidis_JCM_14110 |
| Cas13b-83 | KB291002.1 | | HMPREF9151_01387 | Shmakov et al | 2017 | Prevotella_saccharolytica_F0055 |
| Cas13b-84 | BAKN01000001.1 | | BAKN01000001.1_231 | Shmakov et al | 2017 | Prevotella_saccharolytica_JCM_17484 |
| Cas13b-85 | CP003879.1 | | P700755_002426-2 | Shmakov et al | 2017 | Psychroflexus_torquis_ |
| | | | | | | ATCC_700755 |
| Cas13b-86 | CP007504.1 | | CG09_1718 | Shmakov et al | 2017 | Riemerella_anatipestifer_153 |
| Cas13b-87 | CP007503.1 | | CG08_1741 | Shmakov et al | 2017 | Riemerella_anatipestifer_17 |
| Cas13b-88 | CP002346.1 | | Riean_1551 | Shmakov et al | 2017 | Riemerella_anatipestifer_ATCC_ |
| | | | | | | 11845_DSM_15868 |
| Cas13b-89 | CP003388.1 | | RA0C_1842 | Shmakov et al | 2017 | Riemerella_anatipestifer_ |
| | | | | | | ATCC_11845_DSM_15868 |
| Cas13b-90 | CP004020.1 | | G148_2040 | Shmakov et al | 2017 | Riemerella_anatipestifer_RA_CH_2 |
| Cas13b-91 | CP002562.1 | | RIA_0639 | Shmakov et al | 2017 | Riemerella_anatipestifer_RA_GD |
| Cas13b-92 | KB206042.1 | | KB206042.1_12 | Shmakov et al | 2017 | Riemerella_anatipestifer_RA_SG |
| Cas13b-93 | AENH01000026.1 | | RAYM_05191 | Shmakov et al | 2017 | Riemerella_anatipestifer_RA_YM |
| Cas13b-94 | CP007204.1 | | AS87_08290 | Shmakov et al | 2017 | Riemerella_anatipestifer_Yb2 |
| Cas13c-1 | CCEZ01000008.1 | | CCEZ01000008.1_165 | Shmakov et al | 2017 | Anaerosalibacter_ND1 |
| Cas13c-2 | JTLI01000096.1 | | JTLI01000096.1_1 | Shmakov et al | 2017 | Cetobacterium_ZOR0034_ZOR0034 |
| Cas13c-3 | JAAH01000065.1 | | FUSO8_06265 | Shmakov et al | 2017 | Fusobacterium_necrophorum_DJ_2 |
| Cas13c-4 | JH590847.1 | | HMPREF9466_01873 | Shmakov et al | 2017 | Fusobacterium_necrophorum_ |
| | | | | | | funduliforme_1_1_36S |
| Cas13c-5 | AJSY01000032.1 | | HMPREF1049_0423 | Shmakov et al | 2017 | Fusobacterium_necrophorum_ |
| | | | | | | funduliforme_ATCC_51357 |
| Cas13c-6 | JHXW01000011.1 | | JHXW01000011.1_54 | Shmakov et al | 2017 | Fusobacterium_perfoetens_ |
| | | | | | | ATCC_29250 |
| Cas9-1 | NC_016077 | 352684361 | | Makarova et al | 2015 | Acidaminococcus_intestini_ |
| | | | | | | RyC_MR95_uid74445 |
| Cas9-2 | NC_008578 | 117929158 | | Makarova et al | 2015 | Acidothermus_cellulolyticus_ |
| | | | | | | 11B_uid58501 |
| Cas9-3 | NC_015138 | 326315085 | | Makarova et al | 2015 | Acidovorax_avenae_ATCC_ |
| | | | | | | 19860_uid42497 |
| Cas9-4 | NC_011992 | 222109285 | | Makarova et al | 2015 | Acidovorax_ebreus_TPSY_ |
| | | | | | | uid59233 |
| Cas9-5 | NC_009655 | 152978060 | | Makarova et al | 2015 | Actinobacillus_succinogenes_ |
| | | | | | | 130Z_uid58247 |
| Cas9-6 | NC_018690 | 407692091 | | Makarova et al | 2015 | Actinobacillus_suis_H91_ |
| | | | | | | 0380_uid_176363 |
| Cas9-7 | NC_010655 | 187736489 | | Makarova et al | 2015 | Akkermansia_muciniphila_ |
| | | | | | | ATCC_BAA_835_uid58985 |
| Cas9-8 | NC_014910 | 319760940 | | Makarova et al | 2015 | Alicycliphilus_denitrificans_ |
| | | | | | | BC_uid49953 |
| Cas9-9 | NC_015422 | 330822845 | | Makarova et al | 2015 | Alicycliphilus_denitrificans_ |
| | | | | | | K601_uid66307 |
| Cas9-10 | NC_013854 | 288957741 | | Makarova et al | 2015 | Azospirillum_B510_uid46085 |
| Cas9-11 | NC_022526 | 549484339 | | Makarova et al | 2015 | Bacteroides_CF50_uid222805 |
| Cas9-12 | NC_016776 | 375360193 | | Makarova et al | 2015 | Bacteroides_fragilis_638R_uid84217 |
| Cas9-13 | NC_003228 | 60683389 | | Makarova et al | 2015 | Bacteroides_fragilis_NCTC_ |
| | | | | | | 9343_uid57639 |
| Cas9-14 | NC_020813 | 471261880 | | Makarova et al | 2015 | Bdellovibrio_exovorus_JSS_ |
| | | | | | | uid194119 |
| Cas9-15 | NC_018010 | 390944707 | | Makarova et al | 2015 | Belliella_baltica_DSM_ |
| | | | | | | 15883_uid168182 |
| Cas9-16 | NC_020515 | 470166767 | | Makarova et al | 2015 | Bibersteinia_trehalosi_192_ |
| | | | | | | uid193709 |
| Cas9-17 | NC_014616 | 310286728 | | Makarova et al | 2015 | Bifidobacterium_bifidum_S17_ |
| | | | | | | uid59545 |
| Cas9-18 | NC_013714 | 283456135 | | Makarova et al | 2015 | Bifidobacterium_dentium_ |
| | | | | | | Bd1_uid43091 |
| Cas9-19 | NC_010816 | 189440764 | | Makarova et al | 2015 | Bifidobacterium_longum_ |
| | | | | | | DJO10A_uid58833 |
| Cas9-20 | NC_017221 | 384200944 | | Makarova et al | 2015 | Bifidobacterium_longum_ |
| | | | | | | KACC_91563_uid_158861 |
| Cas9-21 | NC_021031 | 479188345 | | Makarova et al | 2015 | Butyrivibrio_fibrisolvens_ |
| | | | | | | uid197155 |
| Cas9-22 | NC_022362 | 544063172 | | Makarova et al | 2015 | Campylobacter_jejuni_00_ |
| | | | | | | 2425_uid219359 |
| Cas9-23 | NC_022352 | 543948719 | | Makarova et al | 2015 | Campylobacter_jejuni_00_ |
| | | | | | | 2426_uid219324 |
| Cas9-24 | NC_022351 | 543946932 | | Makarova et al | 2015 | Campylobacter_jejuni_00_ |
| | | | | | | 2538_uid219325 |
| Cas9-25 | NC_022353 | 543950499 | | Makarova et al | 2015 | Campylobacter_jejuni_00_ |
| | | | | | | 2544_uid219326 |
| Cas9-26 | NC_022529 | 549693479 | | Makarova et al | 2015 | Campylobacter_jejuni_4031_ |
| | | | | | | uid222817 |
| Cas9-27 | NC_009839 | 157415744 | | Makarova et al | 2015 | Campylobacter_jejuni_81116_ |
| | | | | | | uid58771 |
| Cas9-28 | NC_017279 | 384448746 | | Makarova et al | 2015 | Campylobacter_jejuni_IA3902_ |
| | | | | | | uid159531 |
| Cas9-29 | NC_017280 | 384442102 | | Makarova et al | 2015 | Campylobacter_jejuni_M1_ |
| | | | | | | uid159535 |
| Cas9-30 | NC_017280 | 384442103 | | Makarova et al | 2015 | Campylobacter_jejuni_M1_ |
| | | | | | | uid159535 |
| Cas9-31 | NC_018521 | 403056243 | | Makarova et al | 2015 | Campylobacter_jejuni_NCTC_ |
| | | | | | | 11168_BN148_uid174152 |
| Cas9-32 | NC_002163 | 218563121 | | Makarova et al | 2015 | Campylobacter_jejuni_NCTC_ |
| | | | | | | 11168__ATCC_700819_uid57587 |
| Cas9-33 | NC_018709 | 407942868 | | Makarova et al | 2015 | Campylobacter_jejuni_PT14_ |
| | | | | | | uid176499 |
| Cas9-34 | NC_009707 | 153952471 | | Makarova et al | 2015 | Campylobacter_jejuni_doylei_ |
| | | | | | | 269_97_uid58671 |
| Cas9-35 | NC_014010 | 294086111 | | Makarova et al | 2015 | Candidatus_Puniceispirillum_ |
| | | | | | | marinum_IMCC1322_uid47081 |
| Cas9-36 | NC_015846 | 340622236 | | Makarova et al | 2015 | Capnocytophaga_canimorsus_ |
| | | | | | | Cc5_uid70727 |
| Cas9-37 | NC_011898 | 220930482 | | Makarova et al | 2015 | Clostridium_cellulolyticum_ |
| | | | | | | H10_uid58709 |
| Cas9-38 | NC_021009 | 479136975 | | Makarova et al | 2015 | Coprococcus_catus_ |
| | | | | | | GD_7_uid197174 |
| Cas9-39 | NC_015389 | 328956315 | | Makarova et al | 2015 | Coriobacterium_glomerans_ |
| | | | | | | PW2_uid65787 |
| Cas9-40 | NC_016782 | 375289763 | | Makarova et al | 2015 | Corynebacterium_diphtheriae_ |
| | | | | | | 241_uid83607 |
| Cas9-41 | NC_016799 | 376283539 | | Makarova et al | 2015 | Corynebacterium_diphtheriae_ |
| | | | | | | 31A_uid84309 |
| Cas9-42 | NC_016800 | 376286566 | | Makarova et al | 2015 | Corynebacterium_diphtheriae_ |
| | | | | | | BH8_uid84311 |
| Cas9-43 | NC_016801 | 376289243 | | Makarova et al | 2015 | Corynebacterium_diphtheriae_ |
| | | | | | | C7__beta__uid84313 |
| Cas9-44 | NC_016786 | 376244596 | | Makarova et al | 2015 | Corynebacterium_diphtheriae_ |
| | | | | | | HC01_uid84297 |
| Cas9-45 | NC_016802 | 376292154 | | Makarova et al | 2015 | Corynebacterium_diphtheriae_ |
| | | | | | | HC02_uid84317 |
| Cas9-46 | NC_002935 | 38232678 | | Makarova et al | 2015 | Corynebacterium_diphtheriae_ |
| | | | | | | NCTC_13129_uid57691 |
| Cas9-47 | NC_016790 | 376256051 | | Makarova et al | 2015 | Corynebacterium_diphtheriae_ |
| | | | | | | VA01_uid84305 |
| Cas9-48 | NC_009952 | 159042956 | | Makarova et al | 2015 | Dinoroseobacter_shibae_ |
| | | | | | | DFL_12_uid58707 |
| Cas9-49 | NC_015738 | 339445983 | | Makarova et al | 2015 | Eggerthella_YY7918_uid68707 |
| Cas9-50 | NC_010644 | 187250660 | | Makarova et al | 2015 | Elusimicrobium_minutum_ |
| | | | | | | Pei191_uid58949 |
| Cas9-51 | NC_021023 | 479180325 | | Makarova et al | 2015 | Enterococcus_7L76_ |
| | | | | | | uid197170 |
| Cas9-52 | NC_018221 | 397699066 | | Makarova et al | 2015 | Enterococcus_faecalis_D32_ |
| | | | | | | uid171261 |
| Cas9-53 | NC_017316 | 384512368 | | Makarova et al | 2015 | Enterococcus_faecalis_ |
| | | | | | | OG1RF_uid54927 |
| Cas9-54 | NC_018081 | 392988474 | | Makarova et al | 2015 | Enterococcus_hirac_ATCC_ |
| | | | | | | 9790_uid70619 |
| Cas9-55 | NC_022878 | 558685081 | | Makarova et al | 2015 | Enterococcus_mundtii_ |
| | | | | | | QU_25_uid229420 |
| Cas9-56 | NC_012781 | 238924075 | | Makarova et al | 2015 | Eubacterium_rectale_ |
| | | | | | | ATCC_33656_uid59169 |
| Cas9-57 | NC_017448 | 385789535 | | Makarova et al | 2015 | Fibrobacter_succinogenes_ |
| | | | | | | S85_uid161919 |
| Cas9-58 | NC_013410 | 261414553 | | Makarova et al | 2015 | Fibrobacter_succinogenes_ |
| | | | | | | S85_uid41169 |
| Cas9-59 | NC_016630 | 374307738 | | Makarova et al | 2015 | Filifactor_alocis_ATCC_ |
| | | | | | | 35896_uid46625 |
| Cas9-60 | NC_010376 | 169823755 | | Makarova et al | 2015 | Finegoldia_magna_ATCC_ |
| | | | | | | 29328_uid58867 |
| Cas9-61 | NC_009613 | 150025575 | | Makarova et al | 2015 | Flavobacterium_psychrophilum_ |
| | | | | | | JIP02_86_uid61627 |
| Cas9-62 | NC_015321 | 327405121 | | Makarova et al | 2015 | Fluviicola_taffensis_DSM_ |
| | | | | | | 16823_uid65271 |
| Cas9-63 | NC_017449 | 387824704 | | Makarova et al | 2015 | Francisella_cf__novicida_ |
| | | | | | | 3523_uid162107 |
| Cas9-64 | NC_008601 | 118497352 | | Makarova et al | 2015 | Francisella_novicida_ |
| | | | | | | U112_uid58499 |
| Cas9-65 | NC_009257 | 134302318 | | Makarova et al | 2015 | Francisella_tularensis_WY96_ |
| | | | | | | 3418_uid58811 |
| Cas9-66 | NC_007880 | 89256630 | | Makarova et al | 2015 | Francisella_tularensis_holarctica_ |
| | | | | | | LVS_uid58595 |
| Cas9-67 | NC_007880 | 89256631 | | Makarova et al | 2015 | Francisella_tularensis_holarctica_ |
| | | | | | | LVS_uid58595 |
| Cas9-68 | NC_022196 | 534508854 | | Makarova et al | 2015 | Fusobacterium_3_1_36A2_ |
| | | | | | | uid55995 |
| Cas9-69 | NC_022080 | 530600688 | | Makarova et al | 2015 | Geobacillus_JF8_uid215234 |
| Cas9-70 | NC_011365 | 209542524 | | Makarova et al | 2015 | Gluconacetobacter_diazotrophicus_ |
| | | | | | | PA1_5_uid59075 |
| Cas9-71 | NC_010125 | 162147907 | | Makarova et al | 2015 | Gluconacetobacter_diazotrophicus_ |
| | | | | | | PA1_5_uid61587 |
| Cas9-72 | NC_021021 | 479173968 | | Makarova et al | 2015 | Gordonibacter_pamelaeae_7_ |
| | | | | | | 10_1_b_uid197167 |
| Cas9-73 | NC_015964 | 345430422 | | Makarova et al | 2015 | Haemophilus_parainflucnzae_ |
| | | | | | | T3T1_uid72801 |
| Cas9-74 | NC_020555 | 471315929 | | Makarova et al | 2015 | Helicobacter_cinaedi_ATCC_ |
| | | | | | | BAA_847_uid193765 |
| Cas9-75 | NC_017761 | 386762035 | | Makarova et al | 2015 | Helicobacter_cinaedi_ |
| | | | | | | PAGU611_uid162219 |
| Cas9-76 | NC_013949 | 291276265 | | Makarova et al | 2015 | Helicobacter_mustelae_ |
| | | | | | | 12198_uid46647 |
| Cas9-77 | NC_017464 | 385811609 | | Makarova et al | 2015 | Ignavibacterium_album_ |
| | | | | | | JCM_16511_uid162097 |
| Cas9-78 | NC_014633 | 310780384 | | Makarova et al | 2015 | Ilyobacter_polytropus_ |
| | | | | | | DSM_2926_uid59769 |
| Cas9-79 | NC_015428 | 331702228 | | Makarova et al | 2015 | Lactobacillus_buchneri_ |
| | | | | | | NRRL_B_30929_uid66205 |
| Cas9-80 | NC_018610 | 406027703 | | Makarova et al | 2015 | Lactobacillus_buchneri_ |
| | | | | | | uid73657 |
| Cas9-81 | NC_017474 | 385824065 | | Makarova et al | 2015 | Lactobacillus_casei_BD_II_ |
| | | | | | | uid_162119 |
| Cas9-82 | NC_010999 | 191639137 | | Makarova et al | 2015 | Lactobacillus_casei_BL23_ |
| | | | | | | uid59237 |
| Cas9-83 | NC_017473 | 385820880 | | Makarova et al | 2015 | Lactobacillus_casei_LC2W_ |
| | | | | | | uid162121 |
| Cas9-84 | NC_021721 | 523514789 | | Makarova et al | 2015 | Lactobacillus_casei_ |
| | | | | | | LOCK919_uid210959 |
| Cas9-85 | NC_018641 | 409997999 | | Makarova et al | 2015 | Lactobacillus_casei_ |
| | | | | | | W56_uid178736 |
| Cas9-86 | NC_014334 | 301067199 | | Makarova et al | 2015 | Lactobacillus_casei_Zhang_ |
| | | | | | | uid50673 |
| Cas9-87 | NC_017469 | 385815562 | | Makarova et al | 2015 | Lactobacillus_delbrueckii_ |
| | | | | | | bulgaricus_2038_uid161929 |
| Cas9-88 | NC_017469 | 385815563 | | Makarova et al | 2015 | Lactobacillus_delbrueckii_ |
| | | | | | | bulgaricus_2038_uid161929 |
| Cas9-89 | NC_017469 | 385815564 | | Makarova et al | 2015 | Lactobacillus_delbrueckii_ |
| | | | | | | bulgaricus_2038_uid161929 |
| Cas9-90 | NC_017477 | 385826041 | | Makarova et al | 2015 | Lactobacillus_johnsonii_ |
| | | | | | | DPC_6026_uid162057 |
| Cas9-91 | NC_022112 | 532357525 | | Makarova et al | 2015 | Lactobacillus_paracasei_ |
| | | | | | | 8700_2_uid55295 |
| Cas9-92 | NC_020229 | 448819853 | | Makarova et al | 2015 | Lactobacillus_plantarum_ |
| | | | | | | ZJ316_uid188689 |
| Cas9-93 | NC_017482 | 385828839 | | Makarova et al | 2015 | Lactobacillus_rhamnosus_ |
| | | | | | | GG_uid161983 |
| Cas9-94 | NC_013198 | 258509199 | | Makarova et al | 2015 | Lactobacillus_rhamnosus_ |
| | | | | | | GG_uid59313 |
| Cas9-95 | NC_021723 | 523517690 | | Makarova et al | 2015 | Lactobacillus_rhamnosus_ |
| | | | | | | LOCK900_uid210957 |
| Cas9-96 | NC_017481 | 385839898 | | Makarova et al | 2015 | Lactobacillus_salivarius_ |
| | | | | | | CECT_5713_uid162005 |
| Cas9-97 | NC_017481 | 385839899 | | Makarova et al | 2015 | Lactobacillus_salivarius_ |
| | | | | | | CECT_5713_uid162005 |
| Cas9-98 | NC_017481 | 385839900 | | Makarova et al | 2015 | Lactobacillus_salivarius_ |
| | | | | | | CECT_5713_uid162005 |
| Cas9-99 | NC_007929 | 90961083 | | Makarova et al | 2015 | Lactobacillus_salivarius_ |
| | | | | | | UCC118_uid58233 |
| Cas9-100 | NC_007929 | 90961084 | | Makarova et al | 2015 | Lactobacillus_salivarius_ |
| | | | | | | UCC118_uid58233 |
| Cas9-101 | NC_015978 | 347534532 | | Makarova et al | 2015 | Lactobacillus_sanfranciscensis_ |
| | | | | | | TMW_1_1304_uid72937 |
| Cas9-102 | NC_006368 | 54296138 | | Makarova et al | 2015 | Legionella_pneumophila_ |
| | | | | | | Paris_uid58211 |
| Cas9-103 | NC_018631 | 406600271 | | Makarova et al | 2015 | Leuconostoc_gelidum_JB7_ |
| | | | | | | uid175682 |
| Cas9-104 | NC_003212 | 16801805 | | Makarova et al | 2015 | Listeria_innocua_Clip11262_ |
| | | | | | | uid61567 |
| Cas9-105 | NC_017544 | 386044902 | | Makarova et al | 2015 | Listeria_monocytogenes_ |
| | | | | | | 10403S_uid54461 |
| Cas9-106 | NC_022568 | 550898770 | | Makarova et al | 2015 | Listeria_monocytogenes_ |
| | | | | | | EGD_uid223288 |
| Cas9-107 | NC_017545 | 386048324 | | Makarova et al | 2015 | Listeria_monocytogenes_ |
| | | | | | | J0161_uid54459 |
| Cas9-108 | NC_018586 | 405756714 | | Makarova et al | 2015 | Listeria_monocytogenes_ |
| | | | | | | SLCC2540_uid175106 |
| Cas9-109 | NC_018592 | 404411844 | | Makarova et al | 2015 | Listeria_monocytogenes_ |
| | | | | | | SLCC5850_uid175110 |
| Cas9-110 | NC_018587 | 404282159 | | Makarova et al | 2015 | Listeria_monocytogenes_serotype_ |
| | | | | | | 1_2b_SLCC2755_uid52455 |
| Cas9-111 | NC_018591 | 404287973 | | Makarova et al | 2015 | Listeria_monocytogenes_ |
| | | | | | | serotype_7_SLCC2482_uid174871 |
| Cas9-112 | NC_019949 | 433625054 | | Makarova et al | 2015 | Mycoplasma_cynos_C142_uid184824 |
| Cas9-113 | NC_018412 | 401771107 | | Makarova et al | 2015 | Mycoplasma_gallisepticum_ |
| | | | | | | CA06_2006_052_5_2P_uid172630 |
| Cas9-114 | NC_017503 | 385326554 | | Makarova et al | 2015 | Mycoplasma_gallisepticum_ |
| | | | | | | F_uid162001 |
| Cas9-115 | NC_018407 | 401767318 | | Makarova et al | 2015 | Mycoplasma_gallisepticum_ |
| | | | | | | NC95_13295_2_2P_uid172625 |
| Cas9-116 | NC_018408 | 401768090 | | Makarova et al | 2015 | Mycoplasma_gallisepticum_ |
| | | | | | | NC96_1596_4_2P_uid172626 |
| Cas9-117 | NC_018409 | 401768851 | | Makarova et al | 2015 | Mycoplasma_gallisepticum_ |
| | | | | | | NY01_2001_047_5_1P_uid172627 |
| Cas9-118 | NC_017502 | 385325798 | | Makarova et al | 2015 | Mycoplasma_gallisepticum_ |
| | | | | | | R_high_uid161999 |
| Cas9-119 | NC_004829 | 294660600 | | Makarova et al | 2015 | Mycoplasma_gallisepticum_ |
| | | | | | | R_low__uid57993 |
| Cas9-120 | NC_023030 | 565627373 | | Makarova et al | 2015 | Mycoplasma_gallisepticum_ |
| | | | | | | S6_uid200523 |
| Cas9-121 | NC_018410 | 401769598 | | Makarova et al | 2015 | Mycoplasma_gallisepticum_ |
| | | | | | | WI01_2001_043_13_2P_uid172628 |
| Cas9-122 | NC_006908 | 47458868 | | Makarova et al | 2015 | Mycoplasma_mobile_163K_ |
| | | | | | | uid58077 |
| Cas9-123 | NC_007294 | 71894592 | | Makarova et al | 2015 | Mycoplasma_synoviae_53_ |
| | | | | | | uid58061 |
| Cas9-124 | NC_014752 | 313669044 | | Makarova et al | 2015 | Neisseria_lactamica_020_06_ |
| | | | | | | uid60851 |
| Cas9-125 | NC_010120 | 161869390 | | Makarova et al | 2015 | Neisseria_meningitidis_053442_ |
| | | | | | | uid58587 |
| Cas9-126 | NC_017501 | 385324780 | | Makarova et al | 2015 | Neisseria_meningitidis_8013_ |
| | | | | | | uid161967 |
| Cas9-127 | NC_017512 | 385337435 | | Makarova et al | 2015 | Neisseria_meningitidis_WUE_ |
| | | | | | | 2594_uid162093 |
| Cas9-128 | NC_003116 | 218767588 | | Makarova et al | 2015 | Neisseria_meningitidis_ |
| | | | | | | Z2491_uid57819 |
| Cas9-129 | NC_013016 | 254804356 | | Makarova et al | 2015 | Neisseria_meningitidis_ |
| | | | | | | alpha_14_uid61649 |
| Cas9-130 | NC_014935 | 319957206 | | Makarova et al | 2015 | Nitratifractor_salsuginis_ |
| | | | | | | DSM_16511_uid62183 |
| Cas9-131 | NC_015222 | 325983496 | | Makarova et al | 2015 | Nitrosomonas_AL212_uid55727 |
| Cas9-132 | NC_014363 | 302336020 | | Makarova et al | 2015 | Olsenella_uli_DSM_ |
| | | | | | | 7084_uid51367 |
| Cas9-133 | NC_018016 | 392391493 | | Makarova et al | 2015 | Omithobacterium_rhinotracheale_ |
| | | | | | | DSM_15997_uid168256 |
| Cas9-134 | NC_009719 | 154250555 | | Makarova et al | 2015 | Parvibaculum_lavamentivorans_ |
| | | | | | | DS_1_uid58739 |
| Cas9-135 | NC_002663 | 15602992 | | Makarova et al | 2015 | Pasteurella_multocida_ |
| | | | | | | Pm70_uid57627 |
| Cas9-136 | NC_022780 | 557607382 | | Makarova et al | 2015 | Pediococcus_pentosaceus_ |
| | | | | | | SL4_uid227215 |
| Cas9-137 | NC_017861 | 387132277 | | Makarova et al | 2015 | Prevotella_intermedia_17_ |
| | | | | | | uid163151 |
| Cas9-138 | NC_014033 | 294674019 | | Makarova et al | 2015 | Prevotella_ruminicola_23_ |
| | | | | | | uid47507 |
| Cas9-139 | NC_018721 | 408489713 | | Makarova et al | 2015 | Psychroflexus_torquis_ATCC_ |
| | | | | | | 700755_uid54205 |
| Cas9-140 | NC_007925 | 90425961 | | Makarova et al | 2015 | Rhodopseudomonas_palustris_ |
| | | | | | | BisB18_uid58443 |
| Cas9-141 | NC_007958 | 91975509 | | Makarova et al | 2015 | Rhodopseudomonas_palustris_ |
| | | | | | | BisB5_uid58441 |
| Cas9-142 | NC_007643 | 83591793 | | Makarova et al | 2015 | Rhodospirilluni_rubrum_ATCC_ |
| | | | | | | 11170_uid57655 |
| Cas9-143 | NC_017584 | 386348484 | | Makarova et al | 2015 | Rhodospirilluni_rubrum_ |
| | | | | | | F11_uid162149 |
| Cas9-144 | NC_017045 | 383485594 | | Makarova et al | 2015 | Riemerella_anatipestifer_ATCC_ |
| | | | | | | 11845__DSM_15868_uid159857 |
| Cas9-145 | NC_018609 | 407451859 | | Makarova et al | 2015 | Riemerella_anatipestifer_RA_ |
| | | | | | | CH_1_uid175469 |
| Cas9-146 | NC_020125 | 442314523 | | Makarova et al | 2015 | Riemerella_anatipestifer_RA_ |
| | | | | | | CH_2_uid186548 |
| Cas9-147 | NC_017569 | 386321727 | | Makarova et al | 2015 | Riemerella_anatipestifer_RA_ |
| | | | | | | GD_uid162013 |
| Cas9-148 | NC_021040 | 479204792 | | Makarova et al | 2015 | Roseburia_intestinalis_uid197164 |
| Cas9-149 | NC_020561 | 470213512 | | Makarova et al | 2015 | Sphingomonas_MM_1_uid193771 |
| Cas9-150 | NC_015152 | 325972003 | | Makarova et al | 2015 | Spirochaeta_Buddy_uid63633 |
| Cas9-151 | NC_022998 | 563693590 | | Makarova et al | 2015 | Spiroplasma_apis_B31_uid230613 |
| Cas9-152 | NC_021284 | 507384108 | | Makarova et al | 2015 | Spiroplasma_syrphidicola_ |
| | | | | | | EA_1_uid205054 |
| Cas9-153 | NC_022737 | 556591142 | | Makarova et al | 2015 | Staphylococcus_pasteuri_ |
| | | | | | | SP1_uid226267 |
| Cas9-154 | NC_017568 | 386318630 | | Makarova et al | 2015 | Staphylococcus_pseudintermedius_ |
| | | | | | | ED99_uid162109 |
| Cas9-155 | NC_013515 | 269123826 | | Makarova et al | 2015 | Streptobacillus_moniliformis_ |
| | | | | | | DSM_12112_uid41863 |
| Cas9-156 | NC_022584 | 552737657 | | Makarova et al | 2015 | Streptococcus_I_G2_uid224251 |
| Cas9-157 | NC_021485 | 512539130 | | Makarova et al | 2015 | Streptococcus_agalactiae_ |
| | | | | | | 09mas018883_uid208674 |
| Cas9-158 | NC_004116 | 22537057 | | Makarova et al | 2015 | Streptococcus_agalactiae_ |
| | | | | | | 2603V_R_uid57943 |
| Cas9-159 | NC_021195 | 494703075 | | Makarova et al | 2015 | Streptococcus_agalactiae_2_ |
| | | | | | | 22_uid202215 |
| Cas9-160 | NC_007432 | 76788458 | | Makarova et al | 2015 | Streptococcus_agalactiae_ |
| | | | | | | A909_uid57935 |
| Cas9-161 | NC_018646 | 406709383 | | Makarova et al | 2015 | Streptococcus_agalactiae_ |
| | | | | | | GD201008_001_uid175780 |
| Cas9-162 | NC_021486 | 512544670 | | Makarova et al | 2015 | Streptococcus_agalactiae_ |
| | | | | | | ILRI005_uid208676 |
| Cas9-163 | NC_021507 | 512698372 | | Makarova et al | 2015 | Streptococcus_agalactiae_ |
| | | | | | | ILRI112_uid208675 |
| Cas9-164 | NC_004368 | 25010965 | | Makarova et al | 2015 | Streptococcus_agalactiae_ |
| | | | | | | NEM316_uid61585 |
| Cas9-165 | NC_019048 | 410594450 | | Makarova et al | 2015 | Streptococcus_agalactiae_ |
| | | | | | | SA20_06_uid178722 |
| Cas9-166 | NC_022244 | 538370328 | | Makarova et al | 2015 | Streptococcus_anginosus_ |
| | | | | | | C1051_uid218003 |
| Cas9-167 | NC_019042 | 410494913 | | Makarova et al | 2015 | Streptococcus_dysgalactiae_ |
| | | | | | | equisimilis_AC_2713_uid178644 |
| Cas9-168 | NC_017567 | 386317166 | | Makarova et al | 2015 | Streptococcus_dysgalactiae_ |
| | | | | | | equisimilis_ATCC_12394_uid161979 |
| Cas9-169 | NC_012891 | 251782637 | | Makarova et al | 2015 | Streptococcus_dysgalactiae_ |
| | | | | | | equisimilis_GGS_124_uid59103 |
| Cas9-170 | NC_018712 | 408401787 | | Makarova et al | 2015 | Streptococcus_dysgalactiae_ |
| | | | | | | equisimilis_RE378_uid176684 |
| Cas9-171 | NC_011134 | 195978435 | | Makarova et al | 2015 | Streptococcus_equi_zooepidemicus_ |
| | | | | | | MGCS10565_uid59263 |
| Cas9-172 | NC_017576 | 386338081 | | Makarova et al | 2015 | Streptococcus_gallolyticus_ |
| | | | | | | ATCC_43143_uid162103 |
| Cas9-173 | NC_017576 | 386338091 | | Makarova et al | 2015 | Streptococcus_gallolyticus_ |
| | | | | | | ATCC_43143_uid162103 |
| Cas9-174 | NC_015215 | 325978669 | | Makarova et al | 2015 | Streptococcus_gallolyticus_ |
| | | | | | | ATCC_BAA_2069_uid63617 |
| Cas9-175 | NC_013798 | 288905632 | | Makarova et al | 2015 | Streptococcus_gallolyticus_ |
| | | | | | | UCN34_uid46061 |
| Cas9-176 | NC_013798 | 288905639 | | Makarova et al | 2015 | Streptococcus_gallolyticus_ |
| | | | | | | UCN34_uid46061 |
| Cas9-177 | NC_009785 | 157150687 | | Makarova et al | 2015 | Streptococcus_gordonii_ |
| | | | | | | Challis_substr_CH1_uid57667 |
| Cas9-178 | NC_016826 | 379705580 | | Makarova et al | 2015 | Streptococcus_infantarius_ |
| | | | | | | CJ18_uid87033 |
| Cas9-179 | NC_021314 | 508127396 | | Makarova et al | 2015 | Streptococcus_iniae_ |
| | | | | | | SF1_uid206041 |
| Cas9-180 | NC_021314 | 508127399 | | Makarova et al | 2015 | Streptococcus_iniae_ |
| | | | | | | SF1_uid206041 |
| Cas9-181 | NC_022246 | 538379999 | | Makarova et al | 2015 | Streptococcus_intermedius_ |
| | | | | | | B196_uid218000 |
| Cas9-182 | NC_021900 | 527330434 | | Makarova et al | 2015 | Streptococcus_lutetiensis_ |
| | | | | | | 033_uid213397 |
| Cas9-183 | NC_016749 | 374338350 | | Makarova et al | 2015 | Streptococcus_macedonicus_ |
| | | | | | | ACA_DC_198_uid81631 |
| Cas9-184 | NC_018089 | 397650022 | | Makarova et al | 2015 | Streptococcus_mutans_ |
| | | | | | | GS_5_uid169223 |
| Cas9-185 | NC_017768 | 387785882 | | Makarova et al | 2015 | Streptococcus_mutans_ |
| | | | | | | LJ23_uid162197 |
| Cas9-186 | NC_013928 | 290580220 | | Makarova et al | 2015 | Streptococcus_mutans_ |
| | | | | | | NN2025_uid46353 |
| Cas9-187 | NC_004350 | 24379809 | | Makarova et al | 2015 | Streptococcus_mutans_ |
| | | | | | | UA159_uid57947 |
| Cas9-188 | NC_015600 | 336064611 | | Makarova et al | 2015 | Streptococcus_pasteurianus_ |
| | | | | | | ATCC_43144_uid68019 |
| Cas9-189 | NC_018936 | 410680443 | | Makarova et al | 2015 | Streptococcus_pyogenes_ |
| | | | | | | A20_uid178106 |
| Cas9-190 | NC_020540 | 470200927 | | Makarova et al | 2015 | Streptococcus_pyogenes_ |
| | | | | | | M1_476_uid193766 |
| Cas9-191 | NC_002737 | 15675041 | | Makarova et al | 2015 | Streptococcus_pyogenes_ |
| | | | | | | M1_GAS_uid57845 |
| Cas9-192 | NC_008022 | 94990395 | | Makarova et al | 2015 | Streptococcus_pyogenes_ |
| | | | | | | MGAS10270_uid58571 |
| Cas9-193 | NC_008024 | 94994317 | | Makarova et al | 2015 | Streptococcus_pyogenes_ |
| | | | | | | MGAS10750_uid58575 |
| Cas9-194 | NC_017040 | 383479946 | | Makarova et al | 2015 | Streptococcus_pyogenes_ |
| | | | | | | MGAS15252_uid158037 |
| Cas9-195 | NC_017053 | 383493861 | | Makarova et al | 2015 | Streptococcus_pyogenes_ |
| | | | | | | MGAS1882_uid158061 |
| Cas9-196 | NC_008023 | 94992340 | | Makarova et al | 2015 | Streptococcus_pyogenes_ |
| | | | | | | MGAS2096_uid58573 |
| Cas9-197 | NC_004070 | 21910213 | | Makarova et al | 2015 | Streptococcus_pyogenes_ |
| | | | | | | MGAS315_uid57911 |
| Cas9-198 | NC_007297 | 71910582 | | Makarova et al | 2015 | Streptococcus_pyogenes_ |
| | | | | | | MGAS5005_uid58337 |
| Cas9-199 | NC_007296 | 71903413 | | Makarova et al | 2015 | Streptococcus_pyogenes_ |
| | | | | | | MGAS6180_uid58335 |
| Cas9-200 | NC_008021 | 94988516 | | Makarova et al | 2015 | Streptococcus_pyogenes_ |
| | | | | | | MGAS9429_uid58569 |
| Cas9-201 | NC_011375 | 209559356 | | Makarova et al | 2015 | Streptococcus_pyogenes_ |
| | | | | | | NZ131_uid59035 |
| Cas9-202 | NC_004606 | 28896088 | | Makarova et al | 2015 | Streptococcus_pyogenes_ |
| | | | | | | SSI_1_uid57895 |
| Cas9-203 | NC_017595 | 387783792 | | Makarova et al | 2015 | Streptococcus_salivarius_ |
| | | | | | | JIM8777_uid162145 |
| Cas9-204 | NC_017620 | 386584496 | | Makarova et al | 2015 | Streptococcus_suis_D9_uid162125 |
| Cas9-205 | NC_017950 | 389856936 | | Makarova et al | 2015 | Streptococcus_suis_ST1_uid167482 |
| Cas9-206 | NC_015433 | 330833104 | | Makarova et al | 2015 | Streptococcus_suis_ST3_uid66327 |
| Cas9-207 | NC_006449 | 55822627 | | Makarova et al | 2015 | Streptococcus_thermophilus_ |
| | | | | | | CNRZ1066_uid58221 |
| Cas9-208 | NC_017581 | 386344353 | | Makarova et al | 2015 | Streptococcus_thermophilus_ |
| | | | | | | JIM_8232_uid162157 |
| Cas9-209 | NC_008532 | 116627542 | | Makarova et al | 2015 | Streptococcus_thermophilus_ |
| | | | | | | LMD_9_uid58327 |
| Cas9-210 | NC_008532 | 116628213 | | Makarova et al | 2015 | Streptococcus_thermophilus_ |
| | | | | | | LMD_9_uid58327 |
| Cas9-211 | NC_006448 | 55820735 | | Makarova et al | 2015 | Streptococcus_thermophilus_ |
| | | | | | | LMG_1831_uid58219 |
| Cas9-212 | NC_017927 | 387909441 | | Makarova et al | 2015 | Streptococcus_thermophilus_ |
| | | | | | | MN_ZLW_002_uid166827 |
| Cas9-213 | NC_017927 | 387910220 | | Makarova et al | 2015 | Streptococcus_thermophilus_ |
| | | | | | | MN_ZLW_002_uid166827 |
| Cas9-214 | NC_017563 | 386086348 | | Makarova et al | 2015 | Streptococcus_thermophilus_ |
| | | | | | | ND03_uid162015 |
| Cas9-215 | NC_017563 | 386087120 | | Makarova et al | 2015 | Streptococcus_thermophilus_ |
| | | | | | | ND03_uid162015 |
| Cas9-216 | NC_017958 | 389874754 | | Makarova et al | 2015 | Tistrella_mobilis_KA081020_ |
| | | | | | | 065_uid167486 |
| Cas9-217 | NC_002967 | 42525843 | | Makarova et al | 2015 | Treponema_denticola_ATCC_ |
| | | | | | | 35405_uid57583 |
| Cas9-218 | NC_022097 | 530892607 | | Makarova et al | 2015 | Treponema_pedis_T_A4_ |
| | | | | | | uid215715 |
| Cas9-219 | NC_008786 | 121608211 | | Makarova et al | 2015 | Verminephrobacter_eiseniae_ |
| | | | | | | EF01_2_uid58675 |
| Cas9-220 | NC_021826 | 525888882 | | Makarova et al | 2015 | Vibrio_parahaemolyticus_O1_ |
| | | | | | | K33_CDC_K4557_uid212977 |
| Cas9-221 | NC_021834 | 525913263 | | Makarova et al | 2015 | Vibrio_parahaemolyticus_O1_ |
| | | | | | | K33_CDC_K4557_uid212977 |
| Cas9-222 | NC_021837 | 525919586 | | Makarova et al | 2015 | Vibrio_parahaemolyticus_O1_ |
| | | | | | | K33_CDC_K4557_uid212977 |
| Cas9-223 | NC_021838 | 525927253 | | Makarova et al | 2015 | Vibrio_parahaemolyticus_O1_ |
| | | | | | | K33_CDC_K4557_uid212977 |
| Cas9-224 | NC_015144 | 325955459 | | Makarova et al | 2015 | Weeksella_virosa_DSM_16922_ |
| | | | | | | uid63627 |
| Cas9-225 | NC_005090 | 34557790 | | Makarova et al | 2015 | Wolinella_succinogenes_DSM_ |
| | | | | | | 1740_uid61591 |
| Cas9-226 | NC_005090 | 34557932 | | Makarova et al | 2015 | Wolinella_succinogenes_DSM_ |
| | | | | | | 1740_uid61591 |
| Cas9-227 | NC_014041 | 295136244 | | Makarova et al | 2015 | Zunongwangia_profunda_ |
| | | | | | | SM_A87_uid48073 |
| Cas9-228 | NC_014366 | 304313029 | | Makarova et al | 2015 | gamma_proteobacterium_Hd_ |
| | | | | | | N1_uid51635 |
| Cas9-229 | NC_020419 | 189485058 | | Makarova et al | 2015 | uncultured_Termite_group_1_ |
| | | | | | | bacterium_phylotype_Rs_D17_uid59059 |
| Cas9-230 | NC_020419 | 189485059 | | Makarova et al | 2015 | uncultured_Termite_group_1_ |
| | | | | | | bacterium_phylotype_Rs_D17_uid59059 |
| Cas9-231 | NC_020419 | 189485225 | | Makarova et al | 2015 | uncultured_Termite_group_1_ |
| | | | | | | bacterium_phylotype_Rs_D17_uid59059 |
| Cas9-232 | NC_016001 | 347536497 | | Makarova et al | 2015 | Flavobacterium_branchiophilum_ |
| | | | | | | FL_15_uid73421 |
| Cas9-233 | NC_016510 | 365959402 | | Makarova et al | 2015 | Flavobacterium_columnare_ |
| | | | | | | ATCC_49512_uid80731 |
| Cpf1-1 | NC_012778 | 238917342 | | Makarova et al | 2015 | Eubacterium_eligens_ATCC_ |
| | | | | | | 27750_uid59171 |
| Cpf1-2 | NC_017450 | 385793363 | | Makarova et al | 2015 | Francisella_cf_novicida_Fx_ |
| | | | | | | 1_uid162105 |
| Cpf1-3 | NC_008601 | 118497971 | | Makarova et al | 2015 | Francisella_novicida_U112_ |
| | | | | | | uid58499 |
| Cpf1-4 | NC_010336 | 167627877 | | Makarova et al | 2015 | Francisella_philomiragia_ATCC_ |
| | | | | | | 25017_uid59105 |
| Cpf1-5 | NC_010336 | 167627878 | | Makarova et al | 2015 | Francisella_philomiragia_ATCC_ |
| | | | | | | 25017_uid59105 |
| Cpf1-6 | NC_020913 | 478482906 | | Makarova et al | 2015 | archaeon_Mx1201_uid196597 |

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

Non-limiting embodiments include:

1. A method for identifying a variant of a clustered regularly-interspaced short palindromic repeat (CRISPR) RNA-guided nuclease (RGN) gene of interest comprising:

a) preparing DNA for hybridization from a complex sample comprising a variant of a CRISPR RGN gene of interest, thereby forming a prepared sample DNA comprising said variant of said CRISPR RGN gene of interest;

b) mixing said prepared sample DNA with a labeled bait pool comprising polynucleotide sequences complementary to said CRISPR RGN gene of interest;

c) hybridizing said prepared sample DNA to said labeled bait pool under conditions that allow for hybridization of a labeled bait in said labeled bait pool with said variant of said CRISPR RGN gene of interest to form one or more hybridization complexes comprising captured DNA;

d) sequencing said captured DNA; and

e) analyzing said sequenced captured DNA to identify said variant of said CRISPR RGN gene of interest.

2. The method of embodiment 1, wherein said complex sample is an environmental sample.

3. The method of embodiment 1, wherein said complex sample is a mixed culture of at least two organisms.

4. The method of embodiment 1, wherein said complex sample is a mixed culture of more than two organisms collected from a culture.

5. The method of any one of embodiments 1-4, wherein said labeled baits are specific for at least 10 CRISPR RGN genes of interest.

6. The method of embodiment 5, wherein said labeled baits are specific for at least 300 CRISPR RGN genes of interest.

7. The method of any one of embodiments 1-6, wherein said labeled bait pool comprises at least 1,000, at least 5,000, at least 10,000, at least 20,000, at least 30,000, at least 40,000, or at least 50,000 labeled baits.

8. The method of any of embodiments 1-7, wherein at least 50 distinct labeled baits are mixed with said prepared sample DNA.

9. The method of any one of embodiments 1-8, wherein said labeled baits are 50-200 nt, 70-150 nt, 100-140 nt, or 110-130 nt in length.

10. The method of any one of embodiments 1-9, wherein said labeled baits comprise overlapping labeled baits, said overlapping labeled baits comprising at least two labeled baits that are complementary to a portion of a CRISPR RGN gene of interest, wherein the at least two labeled baits comprise different DNA sequences that are overlapping.

11. The method of embodiment 10, wherein at least 10, at least 30, at least 60, at least 90, or at least 120 nucleotides of each overlapping labeled bait overlap with at least one other overlapping labeled bait.

12. The method of any one of embodiments 1-11, wherein said prepared sample DNA is enriched prior to mixing with said labeled baits.

13. The method of any one of embodiments 1-12, wherein said one or more hybridization complex is captured and purified from unbound prepared sample DNA.

14. The method of embodiment 13, wherein said one or more hybridization complex is captured using a binding partner of said label of said labeled baits attached to a solid phase.

15. The method of embodiment 14, wherein said solid phase is a magnetic bead.

16. The method of any one of embodiments 1-11, wherein steps a), b), and c) are performed using an enrichment kit for multiplex sequencing.

17. The method of any one of embodiments 1-11, wherein captured DNA from said one or more hybridization complex is amplified and index tagged prior to said sequencing.

18. The method of any one of embodiments 1-17, wherein said sequencing comprises multiplex sequencing with gene fragments from different environmental samples.

19. The method of any one of embodiments 1-18, wherein said labeled baits cover each CRISPR RGN gene of interest by at least 2×.

20. The method of any one of embodiments 1-19, wherein said analyzing said sequenced captured DNA comprises performing a sequence similarity search using the sequenced captured DNA against a database of known CRISPR RGN sequences or domains.

21. The method of any one of embodiments 1-19, wherein said analyzing said sequenced captured DNA comprises identifying a full length CRISPR RGN gene sequence of said variant by assembling sequences of said captured DNA and identifying said variant from said full length gene sequence by performing a sequence similarity search using the full length gene sequence against a database of known CRISPR RGN sequences or domains.

22. The method of any one of embodiments 1-21, wherein said variant of said CRISPR RGN gene of interest has less than 95% identity to said CRISPR RGN gene of interest.

23. The method of any one of embodiments 1-22, wherein said labeled bait pool further comprises polynucleotide sequences complementary to sequences flanking said CRISPR RGN gene of interest, and wherein said method further comprises analyzing said sequenced captured DNA for sequences flanking said variant CRISPR RGN gene to identify a sequence encoding a tracrRNA of said variant of said CRISPR RGN gene of interest.

24. The method of embodiment 23, wherein said flanking sequences comprise about 180 nucleotides on either side of said CRISPR RGN gene of interest.

25. The method of embodiment 23 or 24, wherein said labeled baits cover each CRISPR RGN gene of interest and said flanking sequences by at least 2×.

26. The method of any one of embodiments 23-25, wherein analyzing said flanking sequences comprises performing a sequence similarity search using the flanking sequences against a database of known CRISPR tracrRNA sequences.

27. The method of any one of embodiments 23-26, wherein said method further comprises assaying a guide RNA comprising said tracrRNA for binding between the guide RNA and said variant of said CRISPR RGN gene of interest.

28. The method of any one of embodiments 1-22, wherein said method further comprises assaying a guide RNA comprising a crRNA for binding between the guide RNA and said variant of said CRISPR RGN gene of interest.

29. The method of embodiment 27 or 28, wherein said method further comprises identifying a protospacer adjacent motif (PAM) and assaying said variant of said CRISPR RGN gene of interest and said guide RNA for binding to a target nucleotide sequence of interest adjacent to said PAM.

30. The method of embodiment 29, wherein said method further comprises assaying said variant of said CRISPR RGN gene of interest and said guide RNA for cleaving a target nucleotide sequence of interest.

31. A method for preparing an RNA bait pool for the identification of variants of a CRISPR RGN gene of interest comprising:

a) identifying overlapping fragments of a DNA sequence of at least one CRISPR RGN gene of interest, wherein said overlapping fragments span the entire DNA sequence of said CRISPR RGN gene of interest;

b) synthesizing RNA baits complementary to said DNA sequence fragments;

c) labeling said RNA baits with a detectable label; and

d) combining said labeled RNA baits to form said RNA bait pool.

32. The method of embodiment 31, wherein said RNA baits are 50-200 nt, 70-150 nt, 100-140 nt, or 110-130 nt in length.

33. The method of embodiment 31 or 32, wherein said RNA bait pool is specific for at least 10 CRISPR RGN genes of interest.

34. The method of any one of embodiments 31-33, wherein said RNA bait pool comprises at least 1,000, at least 5,000, at least 10,000, at least 20,000, at least 30,000, at least 40,000, or at least 50,000 RNA baits.

35. The method of any one of embodiments 31-34, wherein step a) further comprises obtaining flanking DNA sequences of said at least one CRISPR RGN gene of interest, and wherein said overlapping fragments span the entire DNA sequence of said CRISPR RGN gene of interest and said flanking sequences.

36. The method of embodiment 35, wherein said flanking sequences comprise about 180 nucleotides on either side of said CRISPR RGN gene of interest.

37. A composition comprising the RNA bait pool produced by the method of any one of embodiments 31-36.

38. A composition comprising an RNA bait pool, wherein said RNA bait pool comprises overlapping RNA baits specific for at least one CRISPR RGN gene of interest.

39. The composition of embodiment 38, wherein said RNA baits are 50-200 nt, 70-150 nt, 100-140 nt, or 110-130 nt in length.

40. The composition of embodiment 38 or 39, wherein said RNA bait pool is specific for at least 10 CRISPR RGN genes of interest.

41. The composition of any one of embodiments 38-40, wherein said RNA bait pool comprises at least 1,000, at least 5,000, at least 10,000, at least 20,000, at least 30,000, at least 40,000, or at least 50,000 RNA baits.

42. The composition of any one of embodiments 38-41, wherein said RNA bait pool comprises overlapping RNA baits specific for at least one CRISPR RGN gene of interest and flanking sequences.

43. The composition of embodiment 42, wherein said flanking sequences comprise about 180 nucleotides on either side of said CRISPR RGN gene of interest.

44. A kit comprising an RNA bait pool comprising overlapping RNA baits specific for at least one CRISPR RGN gene of interest and a solid phase, wherein said overlapping RNA baits comprise a detectable label, and wherein a binding partner of said detectable label is attached to said solid phase.

45. The kit of embodiment 44, wherein said RNA baits are 50-200 nt, 70-150 nt, 100-140 nt, or 110-130 nt in length.

46. The kit of embodiment 44 or 45, wherein said RNA bait pool comprises overlapping RNA baits specific for at least one CRISPR RGN gene of interest and flanking sequences.

47. The kit of embodiment 46, wherein said flanking sequences comprise about 180 nucleotides on either side of said CRISPR RGN gene of interest.

The following examples are offered by way of illustration and not by way of limitation.

### EXPERIMENTAL

### Example 1

Sampling and DNA preparation: Samples were collected from diverse environmental niches on private property in NC. Bulk soil samples were suspended in liquid sodium phosphate and plated onto selective media, including: minimal media with 5 ml/L methanol as the primary carbon source, minimal media with 5% NaCl selection (high salt), minimal media incubated in anaerobic conditions, minimal media incubated in aerobic conditions, and selective media for fastidious Gram positive organisms. Genomic DNA was prepared from 400 mg of each sample with the NucleoSpin Soil preparation kit from Clontech. In an alternative method, genomic DNA was prepared with the PowerMax Soil DNA Isolation Kit from Mo Bio Laboratories. Prior to DNA extraction, intact samples were preserved as glycerol stocks for future identification of the organism bearing genes of interest and for retrieval of complete gene sequences. Yields of DNA from soil samples ranged from 66 to 622 micrograms with A260/A280 ratios ranging from 1.81 to 1.93 (Table 2).

TABLE 2
Environmental sources for DNA preparations with yields
and spectrophotometric quality assessments.

| | | DNA | Concen- | | |
|---|---|---|---|---|---|
| | Environmental Sample | Yield | tration | A260/ | A260/ |
| | Description | (μg) | (ng/μl) | A280 | A230 |
| 1 | Anaerobic chick feces | 86 | 45 | 1.77 | 1.70 |
| 2 | Rhizospheric soil | 622 | 350 | 1.85 | 2.10 |
| 3 | Sweet potato soil | 374 | 230 | 1.90 | 2.10 |
| 4 | Bulk soil | 345 | 170 | 1.88 | 1.90 |
| 5 | Anaerobic with methanol | 66 | 35 | 1.81 | 1.80 |
| | selection from soil | | | | |
| 6 | Aerobic with methanol | 540 | 240 | 1.93 | 1.90 |
| | selection from soil | | | | |
| 7 | High salt selection | 106 | 60 | 1.87 | 1.80 |
| | from soil | | | | |

Oligonucleotide baits: Baits for gene capture consisted of approximately 30,000 biotinylated 120 base RNA oligonucleotides that were designed against approximately 330 genes and represent six distinct CRISPR RGN gene families of interest (Table 3). The process is used iteratively such that each subsequent round of hybridization includes baits designed to CRISPR RGN genes discovered in a previous round of gene discovery. In addition to CRISPR RGN genes of interest, additional sequences were included as positive controls (housekeeping genes) and for microbe species identification (16S rRNA). Starting points for baits were staggered at 60 bases to confer 2× coverage for each gene. Baits were synthesized at Agilent with the SureSelect technology. However, additional products for similar use are available from Agilent and other vendors including NimbleGen (SeqCap EZ), Mycroarray (MYbaits), Integrated DNA Technologies (XGen), and LC Sciences (OligoMix).

TABLE 3
Gene families queried in capture reactions with
the number of genes queried for each family.

| Gene Family | # Genes |
|---|---|
| Cas9 | 233 |
| Cas12a | 29 |
| Cas12b | 13 |
| Cas13a | 12 |
| Cas13b | 40 |
| Cas13c | 4 |
| TOTAL | 331 |

TABLE 4
Example baits designed against Streptococcus pyogenes Cas9.

| Base Pair | SEQ | |
|---|---|---|
| Range | ID | Sequence |
| 1 . . . 120 | 1 | TGGATAAGAAATACTCAATAGGCTTAGATATCGGCACAAATAGCGTCGGATGGGCGGT |
| | | GATCACTGATGATTATAAGGTTCCGTCTAAAAAGTTCAAGGTTCTGGGAAATACAGACC |
| | | GCC |
| 40 . . . 159 | 2 | ATAGCGTCGGATGGGCGGTGATCACTGATGATTATAAGGTTCCGTCTAAAAAGTTCAAG |
| | | GTTCTGGGAAATACAGACCGCCACAGTATCAAAAAAAATCTTATAGGGGCTCTTTTATTT |
| | | G |
| 41 . . . 160 | 3 | TAGCGTCGGATGGGCGGTGATCACTGATGATTATAAGGTTCCGTCTAAAAAGTTCAAGG |
| | | TTCTGGGAAATACAGACCGCCACAGTATCAAAAAAAATCTTATAGGGGCTCTTTTATTTG |
| | | A |
| 81 . . . 200 | 4 | CCGTCTAAAAAGTTCAAGGTTCTGGGAAATACAGACCGCCACAGTATCAAAAAAAATCTT |
| | | ATAGGGGCTCTTTTATTTGACAGTGGAGAGATAGCGGAAGCGACTCGTCTCAAACGGAC |
| | | A |
| 121 . . . 240 | 5 | ACAGTATCAAAAAAAATCTTATAGGGGCTCTTTTATTTGACAGTGGAGAGATAGCGGAA |
| | | GCGACTCGTCTCAAACGGACAGCTCGTAGAAGGTATACACGTCGGAAGAATCGTATTTG |
| | | TT |
| 161 . . . 280 | 6 | CAGTGGAGAGATAGCGGAAGCGACTCGTCTCAAACGGACAGCTCGTAGAAGGTATACA |
| | | CGTCGGAAGAATCGTATTTGTTATCTACAGGAGATTTTTTCAAATGAGATGGCGAAAGTA |
| | | GA |
| 200 . . . 319 | 7 | AGCTCGTAGAAGGTATACACGTCGGAAGAATCGTATTTGTTATCTACAGGAGATTTTTTC |
| | | AAATGAGATGGCGAAAGTAGATGATAGTTTCTTTCATCGACTTGAAGAGTCTTTTTTGGT |
| 201 . . . 320 | 8 | GCTCGTAGAAGGTATACACGTCGGAAGAATCGTATTTGTTATCTACAGGAGATTTTTTCA |
| | | AATGAGATGGCGAAAGTAGATGATAGTTTCTTTCATCGACTTGAAGAGTCTTTTTTGGTG |
| 240 . . . 359 | 9 | TATCTACAGGAGATTTTTTCAAATGAGATGGCGAAAGTAGATGATAGTTTCTTTCATCGA |
| | | CTTGAAGAGTCTTTTTTGGTGGAAGAAGACAAGAAGCATGAACGTCATCCTATTTTTGGA |
| 241 . . . 360 | 10 | ATCTACAGGAGATTTTTTCAAATGAGATGGCGAAAGTAGATGATAGTTTCTTTCATCGAC |
| | | TTGAAGAGTCTTTTTTGGTGGAAGAAGACAAGAAGCATGAACGTCATCCTATTTTTGGAA |
| 280 . . . 399 | 11 | ATGATAGTTTCTTTCATCGACTTGAAGAGTCTTTTTTGGTGGAAGAAGACAAGAAGCATG |
| | | AACGTCATCCTATTTTTGGAAATATAGTAGATGAAGTTGCTTATCATGAGAAATATCCAA |
| 281 . . . 400 | 12 | TGATAGTTTCTTTCATCGACTTGAAGAGTCTTTTTTGGTGGAAGAAGACAAGAAGCATGA |
| | | ACGTCATCCTATTTTTGGAAATATAGTAGATGAAGTTGCTTATCATGAGAAATATCCAAC |
| 321 . . . 440 | 13 | GAAGAAGACAAGAAGCATGAACGTCATCCTATTTTTGGAAATATAGTAGATGAAGTTGC |
| | | TTATCATGAGAAATATCCAACTATCTATCATCTGCGAAAAAAATTGGTAGATTCTACTGAT |
| 361 . . . 480 | 14 | ATATAGTAGATGAAGTTGCTTATCATGAGAAATATCCAACTATCTATCATCTGCGAAAAA |
| | | AATTGGTAGATTCTACTGATAAAGCGGATTTGCGCTTAATCTATTTGGCCTTAGCGCATA |
| 401 . . . 520 | 15 | TATCTATCATCTGCGAAAAAAATTGGTAGATTCTACTGATAAAGCGGATTTGCGCTTAAT |
| | | CTATTTGGCCTTAGCGCATATGATTAAGTTTCGTGGTCATTTTTTGATTGAGGGAGATTT |
| 440 . . . 559 | 16 | TAAAGCGGATTTGCGCTTAATCTATTTGGCCTTAGCGCATATGATTAAGTTTCGTGGTCA |
| | | TTTTTTGATTGAGGGAGATTTAAATCCTGATAATAGTGATGTGGACAAACTATTTATCCA |
| 441 . . . 560 | 17 | AAAGCGGATTTGCGCTTAATCTATTTGGCCTTAGCGCATATGATTAAGTTTCGTGGTCAT |
| | | TTTTTGATTGAGGGAGATTTAAATCCTGATAATAGTGATGTGGACAAACTATTTATCCAG |
| 480 . . . 599 | 18 | ATGATTAAGTTTCGTGGTCATTTTTTGATTGAGGGAGATTTAAATCCTGATAATAGTGAT |
| | | GTGGACAAACTATTTATCCAGTTGGTACAAACCTACAATCAATTATTTGAAGAAAACCCT |
| 481 . . . 600 | 19 | TGATTAAGTTTCGTGGTCATTTTTTGATTGAGGGAGATTTAAATCCTGATAATAGTGATG |
| | | TGGACAAACTATTTATCCAGTTGGTACAAACCTACAATCAATTATTTGAAGAAAACCCTA |
| 521 . . . 640 | 20 | AAATCCTGATAATAGTGATGTGGACAAACTATTTATCCAGTTGGTACAAACCTACAATCA |
| | | ATTATTTGAAGAAAACCCTATTAACGCAAGTGGAGTAGATGCTAAAGCGATTCTTTCTGC |
| 561 . . . 680 | 21 | TTGGTACAAACCTACAATCAATTATTTGAAGAAAACCCTATTAACGCAAGTGGAGTAGAT |
| | | GCTAAAGCGATTCTTTCTGCACGATTGAGTAAATCAAGACGATTAGAAAATCTCATTGCT |
| 601 . . . 720 | 22 | TTAACGCAAGTGGAGTAGATGCTAAAGCGATTCTTTCTGCACGATTGAGTAAATCAAGA |
| | | CGATTAGAAAATCTCATTGCTCAGCTCCCCGGTGAGAAGAAAAATGGCTTATTTGGGAA |
| | | TC |
| 641 . . . 760 | 23 | ACGATTGAGTAAATCAAGACGATTAGAAAATCTCATTGCTCAGCTCCCCGGTGAGAAGA |
| | | AAAATGGCTTATTTGGGAATCTCATTGCTTTGTCATTGGGTTTGACCCCTAATTTTAAATC |
| 681 . . . 800 | 24 | CAGCTCCCCGGTGAGAAGAAAAATGGCTTATTTGGGAATCTCATTGCTTTGTCATTGGGT |
| | | TTGACCCCTAATTTTAAATCAAATTTTGATTTGGCAGAAGATGCTAAATTACAGCTTTCA |
| 721 . . . 840 | 25 | TCATTGCTTTGTCATTGGGTTTGACCCCTAATTTTAAATCAAATTTTGATTTGGCAGAAGA |
| | | TGCTAAATTACAGCTTTCAAAAGATACTTACGATGATGATTTAGATAATTTATTGGCGC |
| 760 . . . 879 | 26 | CAAATTTTGATTTGGCAGAAGATGCTAAATTACAGCTTTCAAAAGATACTTACGATGATG |
| | | ATTTAGATAATTTATTGGCGCAAATTGGAGATCAATATGCTGATTTGTTTTTGGCAGCTA |
| 761 . . . 880 | 27 | AAATTTTGATTTGGCAGAAGATGCTAAATTACAGCTTTCAAAAGATACTTACGATGATGA |
| | | TTTAGATAATTTATTGGCGCAAATTGGAGATCAATATGCTGATTTGTTTTTGGCAGCTAA |
| 800 . . . 919 | 28 | AAAAGATACTTACGATGATGATTTAGATAATTTATTGGCGCAAATTGGAGATCAATATGC |
| | | TGATTTGTTTTTGGCAGCTAAGAATTTATCAGATGCTATTTTACTTTCAGATATCCTAAG |
| 801 . . . 920 | 29 | AAAGATACTTACGATGATGATTTAGATAATTTATTGGCGCAAATTGGAGATCAATATGCT |
| | | GATTTGTTTTTGGCAGCTAAGAATTTATCAGATGCTATTTTACTTTCAGATATCCTAAGA |
| 841 . . . 960 | 30 | AAATTGGAGATCAATATGCTGATTTGTTTTTGGCAGCTAAGAATTTATCAGATGCTATTTT |
| | | ACTTTCAGATATCCTAAGATTAAATAGTGAAATAACTAAGGCTCCCCTATCAGCTTCAA |
| 881 . . . 1000 | 31 | GAATTTATCAGATGCTATTTTACTTTCAGATATCCTAAGATTAAATAGTGAAATAACTAAG |
| | | GCTCCCCTATCAGCTTCAATGATTAAACGCTACGATGAACATCATCAAGACTTGACTCT |
| 921 . . . 1040 | 32 | TTAAATAGTGAAATAACTAAGGCTCCCCTATCAGCTTCAATGATTAAACGCTACGATGAA |
| | | CATCATCAAGACTTGACTCTTTTAAAAGCTTTAGTTCGACAACAACTTCCAGAAAAGTAT |
| 960 . . . 1079 | 33 | ATGATTAAACGCTACGATGAACATCATCAAGACTTGACTCTTTTAAAAGCTTTAGTTCGA |
| | | CAACAACTTCCAGAAAAGTATAAAGAAATCTTTTTTGATCAATCAAAAAACGGATATGCA |
| 961 . . . 1080 | 34 | TGATTAAACGCTACGATGAACATCATCAAGACTTGACTCTTTTAAAAGCTTTAGTTCGAC |
| | | AACAACTTCCAGAAAAGTATAAAGAAATCTTTTTTGATCAATCAAAAAACGGATATGCAG |
| 1000 . . . 1119 | 35 | TTTTAAAAGCTTTAGTTCGACAACAACTTCCAGAAAAGTATAAAGAAATCTTTTTTGATCA |
| | | ATCAAAAAACGGATATGCAGGTTATATTGATGGGGGAGCTAGCCAAGAAGAATTTTATA |
| 1001 . . . 1120 | 36 | TTTAAAAGCTTTAGTTCGACAACAACTTCCAGAAAAGTATAAAGAAATCTTTTTTGATCAA |
| | | TCAAAAAACGGATATGCAGGTTATATTGATGGGGGAGCTAGCCAAGAAGAATTTTATAA |
| 1040 . . . 1159 | 37 | TAAAGAAATCTTTTTTGATCAATCAAAAAACGGATATGCAGGTTATATTGATGGGGGAG |
| | | CTAGCCAAGAAGAATTTTATAAATTTATCAAACCAATTTTAGAAAAAATGGATGGTACTG |
| | | A |
| 1041 . . . 1160 | 38 | AAAGAAATCTTTTTTGATCAATCAAAAAACGGATATGCAGGTTATATTGATGGGGGAGC |
| | | TAGCCAAGAAGAATTTTATAAATTTATCAAACCAATTTTAGAAAAAATGGATGGTACTGA |
| | | G |
| 1080 . . . 1199 | 39 | GGTTATATTGATGGGGGAGCTAGCCAAGAAGAATTTTATAAATTTATCAAACCAATTTTA |
| | | GAAAAAATGGATGGTACTGAGGAATTATTGGCGAAACTAAATCGTGAAGATTTGCTGCG |
| | | C |
| 1081 . . . 1200 | 40 | GTTATATTGATGGGGGAGCTAGCCAAGAAGAATTTTATAAATTTATCAAACCAATTTTAG |
| | | AAAAAATGGATGGTACTGAGGAATTATTGGCGAAACTAAATCGTGAAGATTTGCTGCGC |
| | | A |
| 1120 . . . 1239 | 41 | AATTTATCAAACCAATTTTAGAAAAAATGGATGGTACTGAGGAATTATTGGCGAAACTA |
| | | AATCGTGAAGATTTGCTGCGCAAGCAACGGACCTTTGACAACGGCTCTATTCCCCATCAA |
| | | A |
| 1121 . . . 1240 | 42 | ATTTATCAAACCAATTTTAGAAAAAATGGATGGTACTGAGGAATTATTGGCGAAACTAA |
| | | ATCGTGAAGATTTGCTGCGCAAGCAACGGACCTTTGACAACGGCTCTATTCCCCATCAAA |
| | | T |
| 1160 . . . 1279 | 43 | GGAATTATTGGCGAAACTAAATCGTGAAGATTTGCTGCGCAAGCAACGGACCTTTGACA |
| | | ACGGCTCTATTCCCCATCAAATTCACTTGGGTGAGCTGCATGCTATTCTGAGAAGACAAG |
| | | A |
| 1161 . . . 1280 | 44 | GAATTATTGGCGAAACTAAATCGTGAAGATTTGCTGCGCAAGCAACGGACCTTTGACAA |
| | | CGGCTCTATTCCCCATCAAATTCACTTGGGTGAGCTGCATGCTATTCTGAGAAGACAAGA |
| | | A |
| 1200 . . . 1319 | 45 | AAGCAACGGACCTTTGACAACGGCTCTATTCCCCATCAAATTCACTTGGGTGAGCTGCAT |
| | | GCTATTCTGAGAAGACAAGAAGACTTTTATCCATTTTTAAAAGACAATCGTGAGAAGATT |
| 1201 . . . 1320 | 46 | AGCAACGGACCTTTGACAACGGCTCTATTCCCCATCAAATTCACTTGGGTGAGCTGCATG |
| | | CTATTCTGAGAAGACAAGAAGACTTTTATCCATTTTTAAAAGACAATCGTGAGAAGATTG |
| 1241 . . . 1360 | 47 | TCACTTGGGTGAGCTGCATGCTATTCTGAGAAGACAAGAAGACTTTTATCCATTTTTAAA |
| | | AGACAATCGTGAGAAGATTGAAAAAATCTTGACTTTTCGAATTCCTTATTATGTTGGTCC |
| 1280 . . . 1399 | 48 | AGACTTTTATCCATTTTTAAAAGACAATCGTGAGAAGATTGAAAAAATCTTGACTTTTCG |
| | | AATTCCTTATTATGTTGGTCCATTGGCGCGTGGCAATAGTCGTTTTGCATGGATGACTCG |
| 1281 . . . 1400 | 49 | GACTTTTATCCATTTTTAAAAGACAATCGTGAGAAGATTGAAAAAATCTTGACTTTTCGA |
| | | ATTCCTTATTATGTTGGTCCATTGGCGCGTGGCAATAGTCGTTTTGCATGGATGACTCGG |
| 1320 . . . 1439 | 50 | GAAAAAATCTTGACTTTTCGAATTCCTTATTATGTTGGTCCATTGGCGCGTGGCAATAGT |
| | | CGTTTTGCATGGATGACTCGGAAGTCTGAAGAAACAATTACCCCATGGAATTTTGAAGA |
| | | A |
| 1321 . . . 1440 | 51 | AAAAAATCTTGACTTTTCGAATTCCTTATTATGTTGGTCCATTGGCGCGTGGCAATAGTC |
| | | GTTTTGCATGGATGACTCGGAAGTCTGAAGAAACAATTACCCCATGGAATTTTGAAGAA |
| | | G |
| 1360 . . . 1479 | 52 | CATTGGCGCGTGGCAATAGTCGTTTTGCATGGATGACTCGGAAGTCTGAAGAAACAATT |
| | | ACCCCATGGAATTTTGAAGAAGTTGTCGATAAAGGTGCTTCAGCTCAATCATTTATTGAA |
| | | C |
| 1361 . . . 1480 | 53 | ATTGGCGCGTGGCAATAGTCGTTTTGCATGGATGACTCGGAAGTCTGAAGAAACAATTA |
| | | CCCCATGGAATTTTGAAGAAGTTGTCGATAAAGGTGCTTCAGCTCAATCATTTATTGAAC |
| | | G |
| 1400 . . . 1519 | 54 | GAAGTCTGAAGAAACAATTACCCCATGGAATTTTGAAGAAGTTGTCGATAAAGGTGCTT |
| | | CAGCTCAATCATTTATTGAACGCATGACAAACTTTGATAAAAATCTTCCAAATGAAAAAG |
| | | T |
| 1401 . . . 1520 | 55 | AAGTCTGAAGAAACAATTACCCCATGGAATTTTGAAGAAGTTGTCGATAAAGGTGCTTC |
| | | AGCTCAATCATTTATTGAACGCATGACAAACTTTGATAAAAATCTTCCAAATGAAAAAGT |
| | | A |
| 1440 . . . 1559 | 56 | GTTGTCGATAAAGGTGCTTCAGCTCAATCATTTATTGAACGCATGACAAACTTTGATAAA |
| | | AATCTTCCAAATGAAAAAGTACTACCAAAACATAGTTTGCTTTATGAGTATTTTACGGTT |
| 1441 . . . 1560 | 57 | TTGTCGATAAAGGTGCTTCAGCTCAATCATTTATTGAACGCATGACAAACTTTGATAAAA |
| | | ATCTTCCAAATGAAAAAGTACTACCAAAACATAGTTTGCTTTATGAGTATTTTACGGTTT |
| 1480 . . . 1599 | 58 | GCATGACAAACTTTGATAAAAATCTTCCAAATGAAAAAGTACTACCAAAACATAGTTTGC |
| | | TTTATGAGTATTTTACGGTTTATAACGAATTGACAAAGGTCAAATATGTTACTGAGGGAA |
| 1481 . . . 1600 | 59 | CATGACAAACTTTGATAAAAATCTTCCAAATGAAAAAGTACTACCAAAACATAGTTTGCT |
| | | TTATGAGTATTTTACGGTTTATAACGAATTGACAAAGGTCAAATATGTTACTGAGGGAAT |
| 1521 . . . 1640 | 60 | CTACCAAAACATAGTTTGCTTTATGAGTATTTTACGGTTTATAACGAATTGACAAAGGTC |
| | | AAATATGTTACTGAGGGAATGCGAAAACCAGCATTTCTTTCAGGTGAACAGAAGAAAGC |
| | | C |
| 1561 . . . 1680 | 61 | ATAACGAATTGACAAAGGTCAAATATGTTACTGAGGGAATGCGAAAACCAGCATTTCTT |
| | | TCAGGTGAACAGAAGAAAGCCATTGTTGATTTACTCTTCAAAACAAATCGAAAAGTAACC |
| | | G |
| 1600 . . . 1719 | 62 | TGCGAAAACCAGCATTTCTTTCAGGTGAACAGAAGAAAGCCATTGTTGATTTACTCTTCA |
| | | AAACAAATCGAAAAGTAACCGTTAAGCAATTAAAAGAAGATTATTTCAAAAAAATAGAA |
| | | T |
| 1601 . . . 1720 | 63 | GCGAAAACCAGCATTTCTTTCAGGTGAACAGAAGAAAGCCATTGTTGATTTACTCTTCAA |
| | | AACAAATCGAAAAGTAACCGTTAAGCAATTAAAAGAAGATTATTTCAAAAAAATAGAAT |
| | | G |
| 1640 . . . 1759 | 64 | CATTGTTGATTTACTCTTCAAAACAAATCGAAAAGTAACCGTTAAGCAATTAAAAGAAGA |
| | | TTATTTCAAAAAAATAGAATGTTTTGATAGTGTTGAAATTTCAGGAGTTGAAGATAGATT |
| 1641 . . . 1760 | 65 | ATTGTTGATTTACTCTTCAAAACAAATCGAAAAGTAACCGTTAAGCAATTAAAAGAAGAT |
| | | TATTTCAAAAAAATAGAATGTTTTGATAGTGTTGAAATTTCAGGAGTTGAAGATAGATTT |
| 1680 . . . 1799 | 66 | GTTAAGCAATTAAAAGAAGATTATTTCAAAAAAATAGAATGTTTTGATAGTGTTGAAATT |
| | | TCAGGAGTTGAAGATAGATTTAATGCTTCATTAGGTACCTACCATGATTTGCTAAAAATT |
| 1681 . . . 1800 | 67 | TTAAGCAATTAAAAGAAGATTATTTCAAAAAAATAGAATGTTTTGATAGTGTTGAAATTT |
| | | CAGGAGTTGAAGATAGATTTAATGCTTCATTAGGTACCTACCATGATTTGCTAAAAATTA |
| 1721 . . . 1840 | 68 | TTTTGATAGTGTTGAAATTTCAGGAGTTGAAGATAGATTTAATGCTTCATTAGGTACCTA |
| | | CCATGATTTGCTAAAAATTATTAAAGATAAAGATTTTTTGGATAATGAAGAAAATGAAGA |
| 1761 . . . 1880 | 69 | AATGCTTCATTAGGTACCTACCATGATTTGCTAAAAATTATTAAAGATAAAGATTTTTTGG |
| | | ATAATGAAGAAAATGAAGATATCTTAGAGGATATTGTTTTAACATTGACCTTATTTGAA |
| 1801 . . . 1920 | 70 | TTAAAGATAAAGATTTTTTGGATAATGAAGAAAATGAAGATATCTTAGAGGATATTGTTT |
| | | TAACATTGACCTTATTTGAAGATAGGGAGATGATTGAGGAAAGACTTAAAACATATGCT |
| | | C |
| 1840 . . . 1959 | 71 | ATATCTTAGAGGATATTGTTTTAACATTGACCTTATTTGAAGATAGGGAGATGATTGAGG |
| | | AAAGACTTAAAACATATGCTCACCTCTTTGATGATAAGGTGATGAAACAGCTTAAACGTC |
| 1841 . . . 1960 | 72 | TATCTTAGAGGATATTGTTTTAACATTGACCTTATTTGAAGATAGGGAGATGATTGAGGA |
| | | AAGACTTAAAACATATGCTCACCTCTTTGATGATAAGGTGATGAAACAGCTTAAACGTCG |
| 1880 . . . 1999 | 73 | AGATAGGGAGATGATTGAGGAAAGACTTAAAACATATGCTCACCTCTTTGATGATAAGG |
| | | TGATGAAACAGCTTAAACGTCGCCGTTATACTGGTTGGGGACGTTTGTCTCGAAAATTGA |
| | | T |
| 1881 . . . 2000 | 74 | GATAGGGAGATGATTGAGGAAAGACTTAAAACATATGCTCACCTCTTTGATGATAAGGT |
| | | GATGAAACAGCTTAAACGTCGCCGTTATACTGGTTGGGGACGTTTGTCTCGAAAATTGAT |
| | | T |
| 1920 . . . 2039 | 75 | CACCTCTTTGATGATAAGGTGATGAAACAGCTTAAACGTCGCCGTTATACTGGTTGGGG |
| | | ACGTTTGTCTCGAAAATTGATTAATGGTATTAGGGATAAGCAATCTGGCAAAACAATATT |
| | | A |
| 1921 . . . 2040 | 76 | ACCTCTTTGATGATAAGGTGATGAAACAGCTTAAACGTCGCCGTTATACTGGTTGGGGA |
| | | CGTTTGTCTCGAAAATTGATTAATGGTATTAGGGATAAGCAATCTGGCAAAACAATATTA |
| | | G |
| 1960 . . . 2079 | 77 | GCCGTTATACTGGTTGGGGACGTTTGTCTCGAAAATTGATTAATGGTATTAGGGATAAG |
| | | CAATCTGGCAAAACAATATTAGATTTTTTGAAATCAGATGGTTTTGCCAATCGCAATTTTA |
| 1961 . . . 2080 | 78 | CCGTTATACTGGTTGGGGACGTTTGTCTCGAAAATTGATTAATGGTATTAGGGATAAGCA |
| | | ATCTGGCAAAACAATATTAGATTTTTTGAAATCAGATGGTTTTGCCAATCGCAATTTTAT |
| 2000 . . . 2119 | 79 | TAATGGTATTAGGGATAAGCAATCTGGCAAAACAATATTAGATTTTTTGAAATCAGATGG |
| | | TTTTGCCAATCGCAATTTTATGCAGCTGATCCATGATGATAGTTTGACATTTAAAGAAGA |
| 2001 . . . 2120 | 80 | AATGGTATTAGGGATAAGCAATCTGGCAAAACAATATTAGATTTTTTGAAATCAGATGGT |
| | | TTTGCCAATCGCAATTTTATGCAGCTGATCCATGATGATAGTTTGACATTTAAAGAAGAC |
| 2040 . . . 2159 | 81 | GATTTTTTGAAATCAGATGGTTTTGCCAATCGCAATTTTATGCAGCTGATCCATGATGATA |
| | | GTTTGACATTTAAAGAAGACATTCAAAAAGCACAAGTGTCTGGACAAGGCGATAGTTTA |
| 2041 . . . 2160 | 82 | ATTTTTTGAAATCAGATGGTTTTGCCAATCGCAATTTTATGCAGCTGATCCATGATGATAG |
| | | TTTGACATTTAAAGAAGACATTCAAAAAGCACAAGTGTCTGGACAAGGCGATAGTTTAC |
| 2080 . . . 2199 | 83 | TGCAGCTGATCCATGATGATAGTTTGACATTTAAAGAAGACATTCAAAAAGCACAAGTG |
| | | TCTGGACAAGGCGATAGTTTACATGAACATATTGCAAATTTAGCTGGTAGCCCTGCTATT |
| | | A |
| 2081 . . . 2200 | 84 | GCAGCTGATCCATGATGATAGTTTGACATTTAAAGAAGACATTCAAAAAGCACAAGTGT |
| | | CTGGACAAGGCGATAGTTTACATGAACATATTGCAAATTTAGCTGGTAGCCCTGCTATTA |
| | | A |
| 2120 . . . 2239 | 85 | CATTCAAAAAGCACAAGTGTCTGGACAAGGCGATAGTTTACATGAACATATTGCAAATTT |
| | | AGCTGGTAGCCCTGCTATTAAAAAAGGTATTTTACAGACTGTAAAAGTTGTTGATGAATT |
| 2121 . . . 2240 | 86 | ATTCAAAAAGCACAAGTGTCTGGACAAGGCGATAGTTTACATGAACATATTGCAAATTTA |
| | | GCTGGTAGCCCTGCTATTAAAAAAGGTATTTTACAGACTGTAAAAGTTGTTGATGAATTG |
| 2160 . . . 2279 | 87 | CATGAACATATTGCAAATTTAGCTGGTAGCCCTGCTATTAAAAAAGGTATTTTACAGACT |
| | | GTAAAAGTTGTTGATGAATTGGTCAAAGTAATGGGGCGGCATAAGCCAGAAAATATCGT |
| | | T |
| 2161 . . . 2280 | 88 | ATGAACATATTGCAAATTTAGCTGGTAGCCCTGCTATTAAAAAAGGTATTTTACAGACTG |
| | | TAAAAGTTGTTGATGAATTGGTCAAAGTAATGGGGCGGCATAAGCCAGAAAATATCGTT |
| | | A |
| 2200 . . . 2319 | 89 | AAAAAGGTATTTTACAGACTGTAAAAGTTGTTGATGAATTGGTCAAAGTAATGGGGCGG |
| | | CATAAGCCAGAAAATATCGTTATTGAAATGGCACGTGAAAATCAGACAACTCAAAAGGG |
| | | CC |
| 2201 . . . 2320 | 90 | AAAAGGTATTTTACAGACTGTAAAAGTTGTTGATGAATTGGTCAAAGTAATGGGGCGGC |
| | | ATAAGCCAGAAAATATCGTTATTGAAATGGCACGTGAAAATCAGACAACTCAAAAGGGC |
| | | CA |
| 2240 . . . 2359 | 91 | GGTCAAAGTAATGGGGCGGCATAAGCCAGAAAATATCGTTATTGAAATGGCACGTGAA |
| | | AATCAGACAACTCAAAAGGGCCAGAAAAATTCGCGTGAGCGTATGAAACGTATTGAAG |
| | | AAGG |
| 2241 . . . 2360 | 92 | GTCAAAGTAATGGGGCGGCATAAGCCAGAAAATATCGTTATTGAAATGGCACGTGAAA |
| | | ATCAGACAACTCAAAAGGGCCAGAAAAATTCGCGTGAGCGTATGAAACGTATTGAAGA |
| | | AGGT |
| 2281 . . . 2400 | 93 | TTGAAATGGCACGTGAAAATCAGACAACTCAAAAGGGCCAGAAAAATTCGCGTGAGCG |
| | | TATGAAACGTATTGAAGAAGGTATCAAAGAATTAGGAAGTCAGATTCTTAAAGAGCATC |
| | | CTG |
| 2321 . . . 2440 | 94 | GAAAAATTCGCGTGAGCGTATGAAACGTATTGAAGAAGGTATCAAAGAATTAGGAAGT |
| | | CAGATTCTTAAAGAGCATCCTGTTGAAAATACTCAATTGCAAAATGAAAAGCTCTATCTC |
| | | TA |
| 2361 . . . 2480 | 95 | ATCAAAGAATTAGGAAGTCAGATTCTTAAAGAGCATCCTGTTGAAAATACTCAATTGCAA |
| | | AATGAAAAGCTCTATCTCTATTATCTCCAAAATGGAAGAGACATGTATGTGGACCAAGA |
| | | A |
| 2401 . . . 2520 | 96 | TTGAAAATACTCAATTGCAAAATGAAAAGCTCTATCTCTATTATCTCCAAAATGGAAGAG |
| | | ACATGTATGTGGACCAAGAATTAGATATTAATCGTTTAAGTGATTATGATGTCGATCACA |
| 2441 . . . 2560 | 97 | TTATCTCCAAAATGGAAGAGACATGTATGTGGACCAAGAATTAGATATTAATCGTTTAAG |
| | | TGATTATGATGTCGATCACATTGTTCCACAAAGTTTCATTAAAGACGATTCAATAGACAA |
| 2481 . . . 2600 | 98 | TTAGATATTAATCGTTTAAGTGATTATGATGTCGATCACATTGTTCCACAAAGTTTCATTA |
| | | AAGACGATTCAATAGACAATAAGGTCTTAACGCGTTCTGATAAAAATCGTGGTAAATCG |
| 2483 . . . 2602 | 99 | AGATATTAATCGTTTAAGTGATTATGATGTCGATCACATTGTTCCACAAAGTTTCATTAAA |
| | | GACGATTCAATAGACAATAAGGTCTTAACGCGTTCTGATAAAAATCGTGGTAAATCGGA |
| 2521 . . . 2640 | 100 | TTGTTCCACAAAGTTTCATTAAAGACGATTCAATAGACAATAAGGTCTTAACGCGTTCTG |
| | | ATAAAAATCGTGGTAAATCGGATAACGTTCCAAGTGAAGAAGTAGTCAAAAAGATGAAA |
| | | A |
| 2523 . . . 2642 | 101 | GTTCCACAAAGTTTCATTAAAGACGATTCAATAGACAATAAGGTCTTAACGCGTTCTGAT |
| | | AAAAATCGTGGTAAATCGGATAACGTTCCAAGTGAAGAAGTAGTCAAAAAGATGAAAA |
| | | AC |
| 2561 . . . 2680 | 102 | TAAGGTCTTAACGCGTTCTGATAAAAATCGTGGTAAATCGGATAACGTTCCAAGTGAAG |
| | | AAGTAGTCAAAAAGATGAAAAACTATTGGAGACAACTTCTAAATGCCAAGTTAATCACTC |
| | | A |
| 2601 . . . 2720 | 103 | GATAACGTTCCAAGTGAAGAAGTAGTCAAAAAGATGAAAAACTATTGGAGACAACTTCT |
| | | AAATGCCAAGTTAATCACTCAACGTAAGTTTGATAATTTAACGAAAGCTGAACGTGGAG |
| | | GT |
| 2641 . . . 2760 | 104 | ACTATTGGAGACAACTTCTAAATGCCAAGTTAATCACTCAACGTAAGTTTGATAATTTAA |
| | | CGAAAGCTGAACGTGGAGGTTTGAGTGAACTTGATAAAGCTGGTTTTATCAAACGCCAA |
| | | T |
| 2681 . . . 2800 | 105 | ACGTAAGTTTGATAATTTAACGAAAGCTGAACGTGGAGGTTTGAGTGAACTTGATAAAG |
| | | CTGGTTTTATCAAACGCCAATTGGTTGAAACTCGCCAAATCACTAAGCATGTGGCACAAA |
| | | T |
| 2683 . . . 2802 | 106 | GTAAGTTTGATAATTTAACGAAAGCTGAACGTGGAGGTTTGAGTGAACTTGATAAAGCT |
| | | GGTTTTATCAAACGCCAATTGGTTGAAACTCGCCAAATCACTAAGCATGTGGCACAAATT |
| | | T |
| 2684 . . . 2803 | 107 | TAAGTTTGATAATTTAACGAAAGCTGAACGTGGAGGTTTGAGTGAACTTGATAAAGCTG |
| | | GTTTTATCAAACGCCAATTGGTTGAAACTCGCCAAATCACTAAGCATGTGGCACAAATTT |
| | | T |
| 2721 . . . 2840 | 108 | TTGAGTGAACTTGATAAAGCTGGTTTTATCAAACGCCAATTGGTTGAAACTCGCCAAATC |
| | | ACTAAGCATGTGGCACAAATTTTGGATAGTCGCATGAATACTAAATACGATGAAAATGA |
| | | T |
| 2723 . . . 2842 | 109 | GAGTGAACTTGATAAAGCTGGTTTTATCAAACGCCAATTGGTTGAAACTCGCCAAATCAC |
| | | TAAGCATGTGGCACAAATTTTGGATAGTCGCATGAATACTAAATACGATGAAAATGATA |
| | | A |
| 2724 . . . 2843 | 110 | AGTGAACTTGATAAAGCTGGTTTTATCAAACGCCAATTGGTTGAAACTCGCCAAATCACT |
| | | AAGCATGTGGCACAAATTTTGGATAGTCGCATGAATACTAAATACGATGAAAATGATAA |
| | | A |
| 2761 . . . 2880 | 111 | TGGTTGAAACTCGCCAAATCACTAAGCATGTGGCACAAATTTTGGATAGTCGCATGAATA |
| | | CTAAATACGATGAAAATGATAAACTTATTCGAGAGGTTAAAGTGATTACCTTAAAATCTA |
| 2763 . . . 2882 | 112 | GTTGAAACTCGCCAAATCACTAAGCATGTGGCACAAATTTTGGATAGTCGCATGAATACT |
| | | AAATACGATGAAAATGATAAACTTATTCGAGAGGTTAAAGTGATTACCTTAAAATCTAAA |
| 2764 . . . 2883 | 113 | TTGAAACTCGCCAAATCACTAAGCATGTGGCACAAATTTTGGATAGTCGCATGAATACTA |
| | | AATACGATGAAAATGATAAACTTATTCGAGAGGTTAAAGTGATTACCTTAAAATCTAAAT |
| 2801 . . . 2920 | 114 | TTTGGATAGTCGCATGAATACTAAATACGATGAAAATGATAAACTTATTCGAGAGGTTAA |
| | | AGTGATTACCTTAAAATCTAAATTAGTTTCTGACTTCCGAAAAGATTTCCAATTCTATAA |
| 2803 . . . 2922 | 115 | TGGATAGTCGCATGAATACTAAATACGATGAAAATGATAAACTTATTCGAGAGGTTAAA |
| | | GTGATTACCTTAAAATCTAAATTAGTTTCTGACTTCCGAAAAGATTTCCAATTCTATAAAG |
| 2804 . . . 2923 | 116 | GGATAGTCGCATGAATACTAAATACGATGAAAATGATAAACTTATTCGAGAGGTTAAAG |
| | | TGATTACCTTAAAATCTAAATTAGTTTCTGACTTCCGAAAAGATTTCCAATTCTATAAAGT |
| 2841 . . . 2960 | 117 | AAACTTATTCGAGAGGTTAAAGTGATTACCTTAAAATCTAAATTAGTTTCTGACTTCCGAA |
| | | AAGATTTCCAATTCTATAAAGTACGTGAGATTAACAATTACCATCATGCCCATGATGCG |
| 2843 . . . 2962 | 118 | ACTTATTCGAGAGGTTAAAGTGATTACCTTAAAATCTAAATTAGTTTCTGACTTCCGAAAA |
| | | GATTTCCAATTCTATAAAGTACGTGAGATTAACAATTACCATCATGCCCATGATGCGTA |
| 2844 . . . 2963 | 119 | CTTATTCGAGAGGTTAAAGTGATTACCTTAAAATCTAAATTAGTTTCTGACTTCCGAAAA |
| | | GATTTCCAATTCTATAAAGTACGTGAGATTAACAATTACCATCATGCCCATGATGCGTAT |
| 2880 . . . 2999 | 120 | AAATTAGTTTCTGACTTCCGAAAAGATTTCCAATTCTATAAAGTACGTGAGATTAACAATT |
| | | ACCATCATGCCCATGATGCGTATCTTAATGCCGTCGTTGGAACTGCTTTGATTAAGAAA |
| 2881 . . . 3000 | 121 | AATTAGTTTCTGACTTCCGAAAAGATTTCCAATTCTATAAAGTACGTGAGATTAACAATTA |
| | | CCATCATGCCCATGATGCGTATCTTAATGCCGTCGTTGGAACTGCTTTGATTAAGAAAT |
| 2920 . . . 3039 | 122 | AAGTACGTGAGATTAACAATTACCATCATGCCCATGATGCGTATCTTAATGCCGTCGTTG |
| | | GAACTGCTTTGATTAAGAAATATCCAAAACTTGAATCGGAGTTTGTCTATGGTGATTATA |
| 2921 . . . 3040 | 123 | AGTACGTGAGATTAACAATTACCATCATGCCCATGATGCGTATCTTAATGCCGTCGTTGG |
| | | AACTGCTTTGATTAAGAAATATCCAAAACTTGAATCGGAGTTTGTCTATGGTGATTATAA |
| 2961 . . . 3080 | 124 | TATCTTAATGCCGTCGTTGGAACTGCTTTGATTAAGAAATATCCAAAACTTGAATCGGAG |
| | | TTTGTCTATGGTGATTATAAAGTTTATGATGTTCGTAAAATGCTTGCTAAGTCTGAGCAG |
| 3001 . . . 3120 | 125 | ATCCAAAACTTGAATCGGAGTTTGTCTATGGTGATTATAAAGTTTATGATGTTCGTAAAA |
| | | TGCTTGCTAAGTCTGAGCAGGAAATAGGCAAAGCAACCGCAAAATATTTCTTTTACTCTA |
| 3041 . . . 3160 | 126 | AGTTTATGATGTTCGTAAAATGCTTGCTAAGTCTGAGCAGGAAATAGGCAAAGCAACCG |
| | | CAAAATATTTCTTTTACTCTAATATCATGAACTTCTTCAAAACAGAAATTACACTTGCAAA |
| 3080 . . . 3199 | 127 | GGAAATAGGCAAAGCAACCGCAAAATATTTCTTTTACTCTAATATCATGAACTTCTTCAA |
| | | AACAGAAATTACACTTGCAAATGGAGAGATTCGCAAACGCCCTCTAATCGAAACTAATG |
| | | G |
| 3081 . . . 3200 | 128 | GAAATAGGCAAAGCAACCGCAAAATATTTCTTTTACTCTAATATCATGAACTTCTTCAAAA |
| | | CAGAAATTACACTTGCAAATGGAGAGATTCGCAAACGCCCTCTAATCGAAACTAATGGG |
| 3084 . . . 3203 | 129 | ATAGGCAAAGCAACCGCAAAATATTTCTTTTACTCTAATATCATGAACTTCTTCAAAACAG |
| | | AAATTACACTTGCAAATGGAGAGATTCGCAAACGCCCTCTAATCGAAACTAATGGGGAA |
| 3120 . . . 3239 | 130 | AATATCATGAACTTCTTCAAAACAGAAATTACACTTGCAAATGGAGAGATTCGCAAACGC |
| | | CCTCTAATCGAAACTAATGGGGAAACTGGAGAAATTGTCTGGGATAAAGGGCGAGATTT |
| | | T |
| 3121 . . . 3240 | 131 | ATATCATGAACTTCTTCAAAACAGAAATTACACTTGCAAATGGAGAGATTCGCAAACGCC |
| | | CTCTAATCGAAACTAATGGGGAAACTGGAGAAATTGTCTGGGATAAAGGGCGAGATTTT |
| | | G |
| 3124 . . . 3243 | 132 | TCATGAACTTCTTCAAAACAGAAATTACACTTGCAAATGGAGAGATTCGCAAACGCCCTC |
| | | TAATCGAAACTAATGGGGAAACTGGAGAAATTGTCTGGGATAAAGGGCGAGATTTTGC |
| | | CA |
| 3160 . . . 3279 | 133 | ATGGAGAGATTCGCAAACGCCCTCTAATCGAAACTAATGGGGAAACTGGAGAAATTGTC |
| | | TGGGATAAAGGGCGAGATTTTGCCACAGTGCGCAAAGTATTGTCCATGCCCCAAGTCAA |
| | | TA |
| 3161 . . . 3280 | 134 | TGGAGAGATTCGCAAACGCCCTCTAATCGAAACTAATGGGGAAACTGGAGAAATTGTCT |
| | | GGGATAAAGGGCGAGATTTTGCCACAGTGCGCAAAGTATTGTCCATGCCCCAAGTCAAT |
| | | AT |
| 3164 . . . 3283 | 135 | AGAGATTCGCAAACGCCCTCTAATCGAAACTAATGGGGAAACTGGAGAAATTGTCTGGG |
| | | ATAAAGGGCGAGATTTTGCCACAGTGCGCAAAGTATTGTCCATGCCCCAAGTCAATATT |
| | | GT |
| 3200 . . . 3319 | 136 | GGAAACTGGAGAAATTGTCTGGGATAAAGGGCGAGATTTTGCCACAGTGCGCAAAGTA |
| | | TTGTCCATGCCCCAAGTCAATATTGTCAAGAAAACAGAAGTACAGACAGGCGGATTCTC |
| | | CAA |
| 3201 . . . 3320 | 137 | GAAACTGGAGAAATTGTCTGGGATAAAGGGCGAGATTTTGCCACAGTGCGCAAAGTATT |
| | | GTCCATGCCCCAAGTCAATATTGTCAAGAAAACAGAAGTACAGACAGGCGGATTCTCCA |
| | | AG |
| 3204 . . . 3323 | 138 | ACTGGAGAAATTGTCTGGGATAAAGGGCGAGATTTTGCCACAGTGCGCAAAGTATTGTC |
| | | CATGCCCCAAGTCAATATTGTCAAGAAAACAGAAGTACAGACAGGCGGATTCTCCAAGG |
| | | AG |
| 3240 . . . 3359 | 139 | GCCACAGTGCGCAAAGTATTGTCCATGCCCCAAGTCAATATTGTCAAGAAAACAGAAGT |
| | | ACAGACAGGCGGATTCTCCAAGGAGTCAATTTTACCAAAAAGAAATTCGGACAAGCTTA |
| | | TT |
| 3241 . . . 3360 | 140 | CCACAGTGCGCAAAGTATTGTCCATGCCCCAAGTCAATATTGTCAAGAAAACAGAAGTA |
| | | CAGACAGGCGGATTCTCCAAGGAGTCAATTTTACCAAAAAGAAATTCGGACAAGCTTAT |
| | | TG |
| 3244 . . . 3363 | 141 | CAGTGCGCAAAGTATTGTCCATGCCCCAAGTCAATATTGTCAAGAAAACAGAAGTACAG |
| | | ACAGGCGGATTCTCCAAGGAGTCAATTTTACCAAAAAGAAATTCGGACAAGCTTATTGCT |
| | | C |
| 3280 . . . 3399 | 142 | TTGTCAAGAAAACAGAAGTACAGACAGGCGGATTCTCCAAGGAGTCAATTTTACCAAAA |
| | | AGAAATTCGGACAAGCTTATTGCTCGTAAAAAAGACTGGGATCCAAAAAAATATGGTGG |
| | | TT |
| 3281 . . . 3400 | 143 | TGTCAAGAAAACAGAAGTACAGACAGGCGGATTCTCCAAGGAGTCAATTTTACCAAAAA |
| | | GAAATTCGGACAAGCTTATTGCTCGTAAAAAAGACTGGGATCCAAAAAAATATGGTGGT |
| | | TT |
| 3284 . . . 3403 | 144 | CAAGAAAACAGAAGTACAGACAGGCGGATTCTCCAAGGAGTCAATTTTACCAAAAAGAA |
| | | ATTCGGACAAGCTTATTGCTCGTAAAAAAGACTGGGATCCAAAAAAATATGGTGGTTTT |
| | | GA |
| 3320 . . . 3439 | 145 | GGAGTCAATTTTACCAAAAAGAAATTCGGACAAGCTTATTGCTCGTAAAAAAGACTGGG |
| | | ATCCAAAAAAATATGGTGGTTTTGATAGTCCAACGGTAGCTTATTCAGTCCTAGTGGTTG |
| | | C |
| 3321 . . . 3440 | 146 | GAGTCAATTTTACCAAAAAGAAATTCGGACAAGCTTATTGCTCGTAAAAAAGACTGGGA |
| | | TCCAAAAAAATATGGTGGTTTTGATAGTCCAACGGTAGCTTATTCAGTCCTAGTGGTTGC |
| | | T |
| 3324 . . . 3443 | 147 | TCAATTTTACCAAAAAGAAATTCGGACAAGCTTATTGCTCGTAAAAAAGACTGGGATCCA |
| | | AAAAAATATGGTGGTTTTGATAGTCCAACGGTAGCTTATTCAGTCCTAGTGGTTGCTAAG |
| 3360 . . . 3479 | 148 | GCTCGTAAAAAAGACTGGGATCCAAAAAAATATGGTGGTTTTGATAGTCCAACGGTAGC |
| | | TTATTCAGTCCTAGTGGTTGCTAAGGTGGAAAAAGGGAAATCGAAGAAGTTAAAATCCG |
| | | TT |
| 3361 . . . 3480 | 149 | CTCGTAAAAAAGACTGGGATCCAAAAAAATATGGTGGTTTTGATAGTCCAACGGTAGCT |
| | | TATTCAGTCCTAGTGGTTGCTAAGGTGGAAAAAGGGAAATCGAAGAAGTTAAAATCCGT |
| | | TA |
| 3364 . . . 3483 | 150 | GTAAAAAAGACTGGGATCCAAAAAAATATGGTGGTTTTGATAGTCCAACGGTAGCTTAT |
| | | TCAGTCCTAGTGGTTGCTAAGGTGGAAAAAGGGAAATCGAAGAAGTTAAAATCCGTTAA |
| | | AG |
| 3401 . . . 3520 | 151 | TGATAGTCCAACGGTAGCTTATTCAGTCCTAGTGGTTGCTAAGGTGGAAAAAGGGAAAT |
| | | CGAAGAAGTTAAAATCCGTTAAAGAGTTACTAGGGATCACAATTATGGAAAGAAGTTCC |
| | | TT |
| 3404 . . . 3523 | 152 | TAGTCCAACGGTAGCTTATTCAGTCCTAGTGGTTGCTAAGGTGGAAAAAGGGAAATCGA |
| | | AGAAGTTAAAATCCGTTAAAGAGTTACTAGGGATCACAATTATGGAAAGAAGTTCCTTT |
| | | GA |
| 3441 . . . 3560 | 153 | AAGGTGGAAAAAGGGAAATCGAAGAAGTTAAAATCCGTTAAAGAGTTACTAGGGATCA |
| | | CAATTATGGAAAGAAGTTCCTTTGAAAAAAATCCGATTGACTTTTTAGAAGCTAAAGGAT |
| | | AT |
| 3444 . . . 3563 | 154 | GTGGAAAAAGGGAAATCGAAGAAGTTAAAATCCGTTAAAGAGTTACTAGGGATCACAA |
| | | TTATGGAAAGAAGTTCCTTTGAAAAAAATCCGATTGACTTTTTAGAAGCTAAAGGATATA |
| | | AG |
| 3481 . . . 3600 | 155 | AAGAGTTACTAGGGATCACAATTATGGAAAGAAGTTCCTTTGAAAAAAATCCGATTGAC |
| | | TTTTTAGAAGCTAAAGGATATAAGGAAGTTAGAAAAGACTTAATCATTAAACTACCTAAA |
| | | T |
| 3521 . . . 3640 | 156 | TGAAAAAAATCCGATTGACTTTTTAGAAGCTAAAGGATATAAGGAAGTTAGAAAAGACT |
| | | TAATCATTAAACTACCTAAATATAGTCTTTTTGAGTTAGAAAACGGTCGTAAACGGATGC |
| | | T |
| 3561 . . . 3680 | 157 | AAGGAAGTTAGAAAAGACTTAATCATTAAACTACCTAAATATAGTCTTTTTGAGTTAGAA |
| | | AACGGTCGTAAACGGATGCTGGCTAGTGCCGGAGAATTACAAAAAGGAAATGAGCTGG |
| | | CT |
| 3601 . . . 3720 | 158 | ATAGTCTTTTTGAGTTAGAAAACGGTCGTAAACGGATGCTGGCTAGTGCCGGAGAATTA |
| | | CAAAAAGGAAATGAGCTGGCTCTGCCAAGCAAATATGTGAATTTTTTATATTTAGCTAGT |
| | | C |
| 3604 . . . 3723 | 159 | GTCTTTTTGAGTTAGAAAACGGTCGTAAACGGATGCTGGCTAGTGCCGGAGAATTACAA |
| | | AAAGGAAATGAGCTGGCTCTGCCAAGCAAATATGTGAATTTTTTATATTTAGCTAGTCAT |
| | | T |
| 3641 . . . 3760 | 160 | GGCTAGTGCCGGAGAATTACAAAAAGGAAATGAGCTGGCTCTGCCAAGCAAATATGTG |
| | | AATTTTTTATATTTAGCTAGTCATTATGAAAAGTTGAAGGGTAGTCCAGAAGATAACGAA |
| | | CA |
| 3644 . . . 3763 | 161 | TAGTGCCGGAGAATTACAAAAAGGAAATGAGCTGGCTCTGCCAAGCAAATATGTGAATT |
| | | TTTTATATTTAGCTAGTCATTATGAAAAGTTGAAGGGTAGTCCAGAAGATAACGAACAAA |
| | | A |
| 3680 . . . 3799 | 162 | TCTGCCAAGCAAATATGTGAATTTTTTATATTTAGCTAGTCATTATGAAAAGTTGAAGGG |
| | | TAGTCCAGAAGATAACGAACAAAAACAATTGTTTGTGGAGCAGCATAAGCATTATTTAG |
| | | A |
| 3681 . . . 3800 | 163 | CTGCCAAGCAAATATGTGAATTTTTTATATTTAGCTAGTCATTATGAAAAGTTGAAGGGT |
| | | AGTCCAGAAGATAACGAACAAAAACAATTGTTTGTGGAGCAGCATAAGCATTATTTAGA |
| | | T |
| 3684 . . . 3803 | 164 | CCAAGCAAATATGTGAATTTTTTATATTTAGCTAGTCATTATGAAAAGTTGAAGGGTAGT |
| | | CCAGAAGATAACGAACAAAAACAATTGTTTGTGGAGCAGCATAAGCATTATTTAGATGA |
| | | G |
| 3720 . . . 3839 | 165 | CATTATGAAAAGTTGAAGGGTAGTCCAGAAGATAACGAACAAAAACAATTGTTTGTGGA |
| | | GCAGCATAAGCATTATTTAGATGAGATTATTGAGCAAATCAGTGAATTTTCTAAGCGTGT |
| | | T |
| 3721 . . . 3840 | 166 | ATTATGAAAAGTTGAAGGGTAGTCCAGAAGATAACGAACAAAAACAATTGTTTGTGGAG |
| | | CAGCATAAGCATTATTTAGATGAGATTATTGAGCAAATCAGTGAATTTTCTAAGCGTGTT |
| | | A |
| 3724 . . . 3843 | 167 | ATGAAAAGTTGAAGGGTAGTCCAGAAGATAACGAACAAAAACAATTGTTTGTGGAGCA |
| | | GCATAAGCATTATTTAGATGAGATTATTGAGCAAATCAGTGAATTTTCTAAGCGTGTTAT |
| | | TT |
| 3760 . . . 3879 | 168 | AAAAACAATTGTTTGTGGAGCAGCATAAGCATTATTTAGATGAGATTATTGAGCAAATCA |
| | | GTGAATTTTCTAAGCGTGTTATTTTAGCAGATGCCAATTTAGATAAAGTTCTTAGTGCAT |
| 3761 . . . 3880 | 169 | AAAACAATTGTTTGTGGAGCAGCATAAGCATTATTTAGATGAGATTATTGAGCAAATCA |
| | | GTGAATTTTCTAAGCGTGTTATTTTAGCAGATGCCAATTTAGATAAAGTTCTTAGTGCATA |
| 3764 . . . 3883 | 170 | ACAATTGTTTGTGGAGCAGCATAAGCATTATTTAGATGAGATTATTGAGCAAATCAGTGA |
| | | ATTTTCTAAGCGTGTTATTTTAGCAGATGCCAATTTAGATAAAGTTCTTAGTGCATATAA |
| 3800 . . . 3919 | 171 | TGAGATTATTGAGCAAATCAGTGAATTTTCTAAGCGTGTTATTTTAGCAGATGCCAATTT |
| | | AGATAAAGTTCTTAGTGCATATAACAAACATAGAGACAAACCAATACGTGAACAAGCAG |
| | | A |
| 3801 . . . 3920 | 172 | GAGATTATTGAGCAAATCAGTGAATTTTCTAAGCGTGTTATTTTAGCAGATGCCAATTTA |
| | | GATAAAGTTCTTAGTGCATATAACAAACATAGAGACAAACCAATACGTGAACAAGCAGA |
| | | A |
| 3804 . . . 3923 | 173 | ATTATTGAGCAAATCAGTGAATTTTCTAAGCGTGTTATTTTAGCAGATGCCAATTTAGATA |
| | | AAGTTCTTAGTGCATATAACAAACATAGAGACAAACCAATACGTGAACAAGCAGAAAAT |
| 3840 . . . 3959 | 174 | ATTTTAGCAGATGCCAATTTAGATAAAGTTCTTAGTGCATATAACAAACATAGAGACAAA |
| | | CCAATACGTGAACAAGCAGAAAATATTATTCATTTATTTACGTTGACGAATCTTGGAGCT |
| 3841 . . . 3960 | 175 | TTTTAGCAGATGCCAATTTAGATAAAGTTCTTAGTGCATATAACAAACATAGAGACAAAC |
| | | CAATACGTGAACAAGCAGAAAATATTATTCATTTATTTACGTTGACGAATCTTGGAGCTC |
| 3881 . . . 4000 | 176 | TAACAAACATAGAGACAAACCAATACGTGAACAAGCAGAAAATATTATTCATTTATTTAC |
| | | GTTGACGAATCTTGGAGCTCCCACTGCTTTTAAATATTTTGATACAACAATTGATCGTAA |
| 3921 . . . 4040 | 177 | AATATTATTCATTTATTTACGTTGACGAATCTTGGAGCTCCCACTGCTTTTAAATATTTTGA |
| | | TACAACAATTGATCGTAAACGATATACGTCTACAAAAGAAGTTTTAGATGCCACTTTT |
| 3961 . . . 4080 | 178 | CCACTGCTTTTAAATATTTTGATACAACAATTGATCGTAAACGATATACGTCTACAAAAGA |
| | | AGTTTTAGATGCCACTTTTATCCATCAATCCATCACTGGTCTTTATGAAACACGCATTG |
| 3987 . . . 4106 | 179 | ACAATTGATCGTAAACGATATACGTCTACAAAAGAAGTTTTAGATGCCACTTTTATCCATC |
| | | AATCCATCACTGGTCTTTATGAAACACGCATTGATTTGAGTCAGCTAGGAGGTGACTGA |

Gene capture reactions: 3 μg of DNA was used as starting material for the procedure. DNA shearing, capture, post-capture washing and gene amplification are performed in accordance with Agilent SureSelect specifications. Throughout the procedure, DNA is purified with the Agencourt AMPure XP beads, and DNA quality was evaluated with the Agilent TapeStation. Briefly, DNA is sheared to an approximate length of 800 bp using a Covaris Focused-ultrasonicator. In an alternative method, DNA is sheared to lengths from about 400 to about 2000 bp, including about 500 bp, about 600 bp, about 700 bp, about 900 bp, about 1000 bp, about 1200 bp, about 1400 bp, about 1600 bp, about 1800 bp. The Agilent SureSelect Library Prep Kit was used to repair ends, add A bases, ligate the paired-end adaptor and amplify the adaptor-ligated fragments. Prepped DNA samples were lyophilized to contain 750 ng in 3.4 μL and mixed with Agilent SureSelect Hybridization buffers, Capture Library Mix and Block Mix. Hybridization was performed for at least 16 hours at 65° C. In an alternative method, hybridization is performed at a lower temperature (55° C.). DNAs hybridized to biotinylated baits were precipitated with Dynabeads MyOne Streptavidin T1 magnetic beads and washed with SureSelect Binding and Wash Buffers. Captured DNAs were PCR-amplified to add index tags and pooled for multiplexed sequencing.

Genomic DNA libraries were generated by adding a predetermined amount of sample DNA to, for example, the Paired End Sample prep kit PE-102-1001 (ILLUMINA, Inc.) following manufacturer's protocol. Briefly, DNA fragments were generated by random shearing and conjugated to a pair of oligonucleotides in a forked adaptor configuration. The ligated products are amplified using two oligonucleotide primers, resulting in double-stranded blunt-ended products having a different adaptor sequence on either end. The libraries once generated are applied to a flow cell for cluster generation.

Ousters were formed prior to sequencing using the TruSeq PE v3 cluster kit (ILLUMINA, Inc.) following manufacturer's instructions. Briefly, products from a DNA library preparation were denatured and single strands annealed to complementary oligonucleotides on the flow cell surface. A new strand was copied from the original strand in an extension reaction and the original strand was removed by denaturation. The adaptor sequence of the copied strand was annealed to a surface-bound complementary oligonucleotide, forming a bridge and generating a new site for synthesis of a second strand. Multiple cycles of annealing, extension and denaturation in isothermal conditions resulted in growth of clusters, each approximately 1 μm in physical diameter.

The DNA in each cluster was linearized by cleavage within one adaptor sequence and denatured, generating single-stranded template for sequencing by synthesis (SBS) to obtain a sequence read. To perform paired-read sequencing, the products of read 1 can be removed by denaturation, the template was used to generate a bridge, the second strand was re-synthesized and the opposite strand was cleaved to provide the template for the second read. Sequencing was performed using the ILLUMINA, Inc. V4 SBS kit with 100 base paired-end reads on the HiSeq 2000. Briefly, DNA templates were sequenced by repeated cycles of polymerase-directed single base extension. To ensure base-by-base nucleotide incorporation in a stepwise manner, a set of four reversible terminators, A, C, G, and T, each labeled with a different removable fluorophore, was used. The use of modified nucleotides allowed incorporation to be driven essentially to completion without risk of over-incorporation. It also enabled addition of all four nucleotides simultaneously minimizing risk of misincorporation. After each cycle of incorporation, the identity of the inserted base was determined by laser-induced excitation of the fluorophores and fluorescence imaging was recorded. The fluorescent dye and linker were removed to regenerate an available group ready for the next cycle of nucleotide addition. The HiSeq sequencing instrument is designed to perform multiple cycles of sequencing chemistry and imaging to collect sequence data automatically from each cluster on the surface of each lane of an eight-lane flow cell.

Bioinformatics: Sequences were assembled using the CLC Bio suite of bioinformatics tools. The presence of CRISPR RGN genes of interest (Table 3) was determined by BLAST query against a database of those genes of interest. Diversity of organisms present in the sample can be evaluated from 16S identifications. To assess the capacity of this approach for new gene discovery, translations of assembled genes were BLASTed against protein sequences published in public databases including NCBI and PatentLens. The lowest % identity to a gene was 69.98%. Example genes that were captured and sequenced with this method are shown in Table 5.

TABLE 5
Examples of homologs to targeted genes
captured and sequenced with the method.

| | | % | Hit Length |
|---|---|---|---|
| Sequence | Closest Homolog | Identity | (AA) |
| contig_10 - ORF 12 | WP_087094968.1 | 70.65 | 1063 |
| contig_11 - ORF 15 | WP_048723014.1 | 69.98 | 1076 |
| contig_18 - ORF 2 | WP_023519017.1 | 97.74 | 1330 |
| contig_4110 - ORF 21 | WP_065399661.1 | 95.05 | 1090 |
| contig_577 - ORF 9 | WP_076394715.1 | 88.42 | 838 |
| contig_18 - ORF 15 | WP_098836991.1 | 96.35 | 1068 |
| contig_189 - ORF 15 | WP_098135402.1 | 93.09 | 1071 |
| contig_28 - ORF 21 | KXY52240.1 | 96.25 | 1068 |
| contig_5 - ORF 17 | WP_098519598.1 | 94.56 | 1067 |
| contig_78 - ORF 12 | WP_086390158.1 | 94.67 | 1069 |
| contig_17 - ORF 28 | WP_065399661.1 | 95.69 | 1438 |
| contig_53 - ORF 1 | WP_098149203.1 | 88.13 | 1070 |
| contig_1474 - ORF 1 | WP_003343632.1 | 98.63 | 1092 |
| contig_226 - ORF 2 | WP_065399661.1 | 95.69 | 1438 |
| contig_433 - ORF 20 | WP_121730027.1 | 84.99 | 1039 |
| contig_697 - ORF 2 | WP_098149203.1 | 87.94 | 1070 |
| contig_1957 - ORF 1 | WP_002413717.1 | 99.85 | 1337 |

Sequences of the homologs identified in Table 5 were also analyzed for the presence of domains present in known CRISPR RGN genes, including but not limited to RuvC domains, HNH domains, and PAM interacting domains. Results of this analysis are shown in Table 6.

TABLE 6
Protein domains present in captured homologs.

| | | | Domain |
|---|---|---|---|
| | | | Location in |
| Sequence | Domain Name | Database | Protein |
| contig_10 - ORF 12 | Cas9_a | Pfam | 237 . . . 301 |
| contig_10 - ORF 12 | HNH_4 | Pfam | 568 . . . 622 |
| contig_10 - ORF 12 | HNH_CAS9 | PROSITE_PROFILES | 515 . . . 670 |
| contig_10 - ORF 12 | RuvC_III | Pfam | 661 . . . 720 |
| contig_10 - ORF 12 | TIGR01865 | TIGRFAM | 2 . . . 746 |
| contig_11 - ORF 15 | Cas9_a | Pfam | 238 . . . 300 |
| contig_11 - ORF 15 | HNH_4 | Pfam | 568 . . . 622 |
| contig_11 - ORF 15 | HNH_CAS9 | PROSITE_PROFILES | 515 . . . 670 |
| contig_11 - ORF 15 | RuvC_III | Pfam | 661 . . . 782 |
| contig_11 - ORF 15 | TIGR01865 | TIGRFAM | 3 . . . 743 |
| contig_18 - ORF 2 | Cas9-BH | Pfam | 70 . . . 102 |
| contig_18 - ORF 2 | Cas9_PI | Pfam | 1081 . . . 1325 |
| contig_18 - ORF 2 | Cas9_REC | Pfam | 189 . . . 720 |
| contig_18 - ORF 2 | HNH_4 | Pfam | 826 . . . 876 |
| contig_18 - ORF 2 | HNH_CAS9 | PROSITE_PROFILES | 769 . . . 923 |
| contig_18 - ORF 2 | TIGR01865 | TIGRFAM | 12 . . . 1040 |
| contig_4110 - ORF 21 | HNH_4 | Pfam | 479 . . . 529 |
| contig_4110 - ORF 21 | HNH_CAS9 | PROSITE_PROFILES | 418 . . . 590 |
| contig_4110 - ORF 21 | RuvC_III | Pfam | 580 . . . 786 |
| contig_577 - ORF 9 | HNH_4 | Pfam | 200 . . . 252 |
| contig_577 - ORF 9 | HNH_CAS9 | PROSITE_PROFILES | 145 . . . 304 |
| contig_577 - ORF 9 | RuvC_III | Pfam | 294 . . . 472 |
| contig_18 - ORF 15 | HNH_4 | Pfam | 560 . . . 614 |
| contig_18 - ORF 15 | HNH_CAS9 | PROSITE_PROFILES | 509 . . . 662 |
| contig_18 - ORF 15 | RuvC_III | Pfam | 654 . . . 712 |
| contig_18 - ORF 15 | TIGR01865 | TIGRFAM | 3 . . . 747 |
| contig_189 - ORF 15 | HNH_4 | Pfam | 574 . . . 636 |
| contig_189 - ORF 15 | HNH_CAS9 | PROSITE_PROFILES | 523 . . . 685 |
| contig_189 - ORF 15 | RuvC_III | Pfam | 678 . . . 776 |
| contig_189 - ORF 15 | TIGR01865 | TIGRFAM | 5 . . . 773 |
| contig_28 - ORF 21 | HNH_4 | Pfam | 566 . . . 620 |
| contig_28 - ORF 21 | HNH_CAS9 | PROSITE_PROFILES | 515 . . . 668 |
| contig_28 - ORF 21 | RuvC_III | Pfam | 660 . . . 776 |
| contig_28 - ORF 21 | TIGR01865 | TIGRFAM | 8 . . . 755 |
| contig_5 - ORF 17 | Cytoplasmic domain | PHOBIUS | 1 . . . 6 |
| contig_5 - ORF 17 | HNH_4 | Pfam | 566 . . . 620 |
| contig_5 - ORF 17 | HNH_CAS9 | PROSITE_PROFILES | 515 . . . 668 |
| contig_5 - ORF 17 | Non cytoplasmic domain | PHOBIUS | 26 . . . 1073 |
| contig_5 - ORF 17 | RuvC_III | Pfam | 660 . . . 759 |
| contig_5 - ORF 17 | TIGR01865 | TIGRFAM | 8 . . . 754 |
| contig_5 - ORF 17 | Transmembrane region | PHOBIUS | 7 . . . 25 |
| contig_78 - ORF 12 | HNH_4 | Pfam | 559 . . . 613 |
| contig_78 - ORF 12 | HNH_CAS9 | PROSITE_PROFILES | 508 . . . 662 |
| contig_78 - ORF 12 | TIGR01865 | TIGRFAM | 3 . . . 741 |
| contig_17 - ORF 28 | Cas9-BH | Pfam | 62 . . . 96 |
| contig_17 - ORF 28 | HNH_4 | Pfam | 829 . . . 879 |
| contig_17 - ORF 28 | HNH_CAS9 | PROSITE_PROFILES | 768 . . . 940 |
| contig_17 - ORF 28 | RuvC_III | Pfam | 930 . . . 1136 |
| contig_53 - ORF 1 | HNH_4 | Pfam | 574 . . . 636 |
| contig_53 - ORF 1 | HNH_CAS9 | PROSITE_PROFILES | 523 . . . 685 |
| contig_53 - ORF 1 | RuvC_III | Pfam | 680 . . . 739 |
| contig_53 - ORF 1 | TIGR01865 | TIGRFAM | 5 . . . 772 |
| contig_1474 - ORF 1 | Cas9_a | Pfam | 237 . . . 311 |
| contig_1474 - ORF 1 | Cas9_REC | Pfam | 233 . . . 406 |
| contig_1474 - ORF 1 | HNH_4 | Pfam | 562 . . . 616 |
| contig_1474 - ORF 1 | HNH_CAS9 | PROSITE_PROFILES | 511 . . . 665 |
| contig_1474 - ORF 1 | RuvC_III | Pfam | 659 . . . 751 |
| contig_1474 - ORF 1 | TIGR01865 | TIGRFAM | 3 . . . 768 |
| contig_226 - ORF 2 | Cas9-BH | Pfam | 62 . . . 96 |
| contig_226 - ORF 2 | HNH_4 | Pfam | 829 . . . 879 |
| contig_226 - ORF 2 | HNH_CAS9 | PROSITE_PROFILES | 768 . . . 940 |
| contig_226 - ORF 2 | RuvC_III | Pfam | 930 . . . 1136 |
| contig_433 - ORF 20 | HNH_4 | Pfam | 623 . . . 676 |
| contig_433 - ORF 20 | HNH_CAS9 | PROSITE_PROFILES | 564 . . . 727 |
| contig_433 - ORF 20 | RuvC_III | Pfam | 719 . . . 811 |
| contig_433 - ORF 20 | TIGR01865 | TIGRFAM | 523 . . . 812 |
| contig_697 - ORF 2 | HNH_4 | Pfam | 574 . . . 636 |
| contig_697 - ORF 2 | HNH_CAS9 | PROSITE_PROFILES | 523 . . . 685 |
| contig_697 - ORF 2 | RuvC_III | Pfam | 680 . . . 739 |
| contig_697 - ORF 2 | TIGR01865 | TIGRFAM | 5 . . . 772 |
| contig_1957 - ORF 1 | Cas9-BH | Pfam | 62 . . . 93 |
| contig_1957 - ORF 1 | Cas9_PI | Pfam | 1086 . . . 1331 |
| contig_1957 - ORF 1 | Cas9_REC | Pfam | 181 . . . 724 |
| contig_1957 - ORF 1 | HNH_4 | Pfam | 832 . . . 882 |
| contig_1957 - ORF 1 | HNH_CAS9 | PROSITE_PROFILES | 781 . . . 932 |
| contig_1957 - ORF 1 | TIGR01865 | TIGRFAM | 4 . . . 1046 |

Guide RNA Confirmation: To identify tracrRNA-coding regions, Hidden Markov Models (HMMs) of RNA structures and sequences are developed using previously published tracrRNAs (see, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety) as well as internal validated sequences. The HMM profile is used to predict the coding region for the tracrRNA. The corresponding crRNA is predicted by designing crRNAs that are partially complementary to the anti-repeat region of the tracrRNA, and to establish the functional modules seen in guide RNAs, including the lower stem, bulge, and upper stem. To verify that the newly identified RGN can bind the predicted crRNA, and in some embodiments, tracrRNA, a protein binding assay is performed. In one particular assay, RNAs labeled with a detectable label, such as biotin, are incubated with the RGN. The guide RNA is then pulled down with a binding partner of the detectable label (e.g., avidin) to pulldown bound RGN proteins. Confirmation of the binding can be visualized via SDS-PAGE or Western blot with antibodies that recognize the RGN protein or a detectable label bound to the RGN protein.

1. A method for identifying a variant of a clustered regularly-interspaced short palindromic repeat (CRISPR) RNA-guided nuclease (RGN) gene of interest comprising:
a) preparing DNA for hybridization from a complex sample comprising a variant of a CRISPR RGN gene of interest, thereby forming a prepared sample DNA comprising said variant of said CRISPR RGN gene of interest;
b) mixing said prepared sample DNA with a labeled bait pool comprising polynucleotide sequences complementary to said CRISPR RGN gene of interest;
c) hybridizing said prepared sample DNA to said labeled bait pool under conditions that allow for hybridization of a labeled bait in said labeled bait pool with said variant of said CRISPR RGN gene of interest to form one or more hybridization complexes comprising captured DNA;
d) sequencing said captured DNA; and
e) analyzing said sequenced captured DNA to identify said variant of said CRISPR RGN gene of interest.

2. The method of claim 1, wherein said complex sample is an environmental sample.

3. The method of claim 1, wherein said complex sample is a mixed culture of at least two organisms.

4. (canceled)

5. The method of claim 1, wherein said labeled baits are specific for at least 10 CRISPR RGN genes of interest.

6. The method of claim 5, wherein said labeled baits are specific for at least 300 CRISPR RGN genes of interest.

7. The method of claim 1, wherein said labeled bait pool comprises at least 1,000, at least 5,000, at least 10,000, at least 20,000, at least 30,000, at least 40,000, or at least 50,000 labeled baits.

8. The method of claim 1, wherein at least 50 distinct labeled baits are mixed with said prepared sample DNA.

9. The method of claim 1, wherein said labeled baits are 50-200 nt, 70-150 nt, 100-140 nt, or 110-130 nt in length.

10. The method of claim 1, wherein said labeled baits comprise overlapping labeled baits, said overlapping labeled baits comprising at least two labeled baits that are complementary to a portion of a CRISPR RGN gene of interest, wherein the at least two labeled baits comprise different DNA sequences that are overlapping.

11. The method of claim 10, wherein at least 10, at least 30, at least 60, at least 90, or at least 120 nucleotides of each overlapping labeled bait overlap with at least one other overlapping labeled bait.

12. The method of claim 1, wherein said prepared sample DNA is enriched prior to mixing with said labeled baits.

13. The method of claim 1, wherein said one or more hybridization complex is captured and purified from unbound prepared sample DNA.

14. The method of claim 13, wherein said one or more hybridization complex is captured using a binding partner of said label of said labeled baits attached to a solid phase.

15. (canceled)

16. (canceled)

17. The method of claim 1, wherein captured DNA from said one or more hybridization complex is amplified and index tagged prior to said sequencing.

18. (canceled)

19. (canceled)

20. The method of claim 1, wherein said analyzing said sequenced captured DNA comprises performing a sequence similarity search using the sequenced captured DNA against a database of known CRISPR RGN sequences or domains.

21. (canceled)

22. (canceled)

23. The method of claim 1, wherein said labeled bait pool further comprises polynucleotide sequences complementary to sequences flanking said CRISPR RGN gene of interest, and wherein said method further comprises analyzing said sequenced captured DNA for sequences flanking said variant CRISPR RGN gene to identify a sequence encoding a tracrRNA of said variant of said CRISPR RGN gene of interest.

24. The method of claim 23, wherein said flanking sequences comprise about 180 nucleotides on either side of said CRISPR RGN gene of interest.

25. (canceled)

26. The method of claim 23, wherein analyzing said flanking sequences comprises performing a sequence similarity search using the flanking sequences against a database of known CRISPR tracrRNA sequences.

27. (canceled)

28. The method of claim 1, wherein said method further comprises assaying a guide RNA comprising a crRNA for binding between the guide RNA and said variant of said CRISPR RGN gene of interest.

29. The method of claim 28, wherein said method further comprises identifying a protospacer adjacent motif (PAM) and assaying said variant of said CRISPR RGN gene of interest and said guide RNA for binding to a target nucleotide sequence of interest adjacent to said PAM.

30-47. (canceled)


SEQUENCE LISTING (179 sequences)

SEQ ID NO: 1 - DNA, 120 bp, Streptococcus pyogenes
tggataagaaatactcaataggcttagatatcggcacaaatagcgtcggatgggcggtgatcactgatgattataaggttccgtctaaaaagttcaaggttctgggaaatacagaccgcc

SEQ ID NO: 2 - DNA, 120 bp, Streptococcus pyogenes
atagcgtcggatgggcggtgatcactgatgattataaggttccgtctaaaaagttcaaggttctgggaaatacagaccgccacagtatcaaaaaaaatcttataggggctcttttatttg

SEQ ID NO: 3 - DNA, 120 bp, Streptococcus pyogenes
tagcgtcggatgggcggtgatcactgatgattataaggttccgtctaaaaagttcaaggttctgggaaatacagaccgccacagtatcaaaaaaaatcttataggggctcttttatttga

SEQ ID NO: 4 - DNA, 120 bp, Streptococcus pyogenes
ccgtctaaaaagttcaaggttctgggaaatacagaccgccacagtatcaaaaaaaatcttataggggctcttttatttgacagtggagagatagcggaagcgactcgtctcaaacggaca

SEQ ID NO: 5 - DNA, 120 bp, Streptococcus pyogenes
acagtatcaaaaaaaatcttataggggctcttttatttgacagtggagagatagcggaagcgactcgtctcaaacggacagctcgtagaaggtatacacgtcggaagaatcgtatttgtt

SEQ ID NO: 6 - DNA, 120 bp, Streptococcus pyogenes
cagtggagagatagcggaagcgactcgtctcaaacggacagctcgtagaaggtatacacgtcggaagaatcgtatttgttatctacaggagattttttcaaatgagatggcgaaagtaga

SEQ ID NO: 7 - DNA, 120 bp, Streptococcus pyogenes
agctcgtagaaggtatacacgtcggaagaatcgtatttgttatctacaggagattttttcaaatgagatggcgaaagtagatgatagtttctttcatcgacttgaagagtcttttttggt

SEQ ID NO: 8 - DNA, 120 bp, Streptococcus pyogenes
gctcgtagaaggtatacacgtcggaagaatcgtatttgttatctacaggagattttttcaaatgagatggcgaaagtagatgatagtttctttcatcgacttgaagagtcttttttggtg

SEQ ID NO: 9 - DNA, 120 bp, Streptococcus pyogenes
tatctacaggagattttttcaaatgagatggcgaaagtagatgatagtttctttcatcgacttgaagagtcttttttggtggaagaagacaagaagcatgaacgtcatcctatttttgga

SEQ ID NO: 10 - DNA, 120 bp, Streptococcus pyogenes
atctacaggagattttttcaaatgagatggcgaaagtagatgatagtttctttcatcgacttgaagagtcttttttggtggaagaagacaagaagcatgaacgtcatcctatttttggaa

SEQ ID NO: 11 - DNA, 120 bp, Streptococcus pyogenes
atgatagtttctttcatcgacttgaagagtcttttttggtggaagaagacaagaagcatgaacgtcatcctatttttggaaatatagtagatgaagttgcttatcatgagaaatatccaa

SEQ ID NO: 12 - DNA, 120 bp, Streptococcus pyogenes
tgatagtttctttcatcgacttgaagagtcttttttggtggaagaagacaagaagcatgaacgtcatcctatttttggaaatatagtagatgaagttgcttatcatgagaaatatccaac

SEQ ID NO: 13 - DNA, 120 bp, Streptococcus pyogenes
gaagaagacaagaagcatgaacgtcatcctatttttggaaatatagtagatgaagttgcttatcatgagaaatatccaactatctatcatctgcgaaaaaaattggtagattctactgat

SEQ ID NO: 14 - DNA, 120 bp, Streptococcus pyogenes
atatagtagatgaagttgcttatcatgagaaatatccaactatctatcatctgcgaaaaaaattggtagattctactgataaagcggatttgcgcttaatctatttggccttagcgcata

SEQ ID NO: 15 - DNA, 120 bp, Streptococcus pyogenes
tatctatcatctgcgaaaaaaattggtagattctactgataaagcggatttgcgcttaatctatttggccttagcgcatatgattaagtttcgtggtcattttttgattgagggagattt

SEQ ID NO: 16 - DNA, 120 bp, Streptococcus pyogenes
taaagcggatttgcgcttaatctatttggccttagcgcatatgattaagtttcgtggtcattttttgattgagggagatttaaatcctgataatagtgatgtggacaaactatttatcca

SEQ ID NO: 17 - DNA, 120 bp, Streptococcus pyogenes
aaagcggatttgcgcttaatctatttggccttagcgcatatgattaagtttcgtggtcattttttgattgagggagatttaaatcctgataatagtgatgtggacaaactatttatccag

SEQ ID NO: 18 - DNA, 120 bp, Streptococcus pyogenes
atgattaagtttcgtggtcattttttgattgagggagatttaaatcctgataatagtgatgtggacaaactatttatccagttggtacaaacctacaatcaattatttgaagaaaaccct

SEQ ID NO: 19 - DNA, 120 bp, Streptococcus pyogenes
tgattaagtttcgtggtcattttttgattgagggagatttaaatcctgataatagtgatgtggacaaactatttatccagttggtacaaacctacaatcaattatttgaagaaaacccta

SEQ ID NO: 20 - DNA, 120 bp, Streptococcus pyogenes
aaatcctgataatagtgatgtggacaaactatttatccagttggtacaaacctacaatcaattatttgaagaaaaccctattaacgcaagtggagtagatgctaaagcgattctttctgc

SEQ ID NO: 21 - DNA, 120 bp, Streptococcus pyogenes
ttggtacaaacctacaatcaattatttgaagaaaaccctattaacgcaagtggagtagatgctaaagcgattctttctgcacgattgagtaaatcaagacgattagaaaatctcattgct

SEQ ID NO: 22 - DNA, 120 bp, Streptococcus pyogenes
ttaacgcaagtggagtagatgctaaagcgattctttctgcacgattgagtaaatcaagacgattagaaaatctcattgctcagctccccggtgagaagaaaaatggcttatttgggaatc

SEQ ID NO: 23 - DNA, 120 bp, Streptococcus pyogenes
acgattgagtaaatcaagacgattagaaaatctcattgctcagctccccggtgagaagaaaaatggcttatttgggaatctcattgctttgtcattgggtttgacccctaattttaaatc

SEQ ID NO: 24 - DNA, 120 bp, Streptococcus pyogenes
cagctccccggtgagaagaaaaatggcttatttgggaatctcattgctttgtcattgggtttgacccctaattttaaatcaaattttgatttggcagaagatgctaaattacagctttca

SEQ ID NO: 25 - DNA, 120 bp, Streptococcus pyogenes
tcattgctttgtcattgggtttgacccctaattttaaatcaaattttgatttggcagaagatgctaaattacagctttcaaaagatacttacgatgatgatttagataatttattggcgc

SEQ ID NO: 26 - DNA, 120 bp, Streptococcus pyogenes
caaattttgatttggcagaagatgctaaattacagctttcaaaagatacttacgatgatgatttagataatttattggcgcaaattggagatcaatatgctgatttgtttttggcagcta

SEQ ID NO: 27 - DNA, 120 bp, Streptococcus pyogenes
aaattttgatttggcagaagatgctaaattacagctttcaaaagatacttacgatgatgatttagataatttattggcgcaaattggagatcaatatgctgatttgtttttggcagctaa

SEQ ID NO: 28 - DNA, 120 bp, Streptococcus pyogenes
aaaagatacttacgatgatgatttagataatttattggcgcaaattggagatcaatatgctgatttgtttttggcagctaagaatttatcagatgctattttactttcagatatcctaag

SEQ ID NO: 29 - DNA, 120 bp, Streptococcus pyogenes
aaagatacttacgatgatgatttagataatttattggcgcaaattggagatcaatatgctgatttgtttttggcagctaagaatttatcagatgctattttactttcagatatcctaaga

SEQ ID NO: 30 - DNA, 120 bp, Streptococcus pyogenes
aaattggagatcaatatgctgatttgtttttggcagctaagaatttatcagatgctattttactttcagatatcctaagattaaatagtgaaataactaaggctcccctatcagcttcaa

SEQ ID NO: 31 - DNA, 120 bp, Streptococcus pyogenes
gaatttatcagatgctattttactttcagatatcctaagattaaatagtgaaataactaaggctcccctatcagcttcaatgattaaacgctacgatgaacatcatcaagacttgactct

SEQ ID NO: 32 - DNA, 120 bp, Streptococcus pyogenes
ttaaatagtgaaataactaaggctcccctatcagcttcaatgattaaacgctacgatgaacatcatcaagacttgactcttttaaaagctttagttcgacaacaacttccagaaaagtat

SEQ ID NO: 33 - DNA, 120 bp, Streptococcus pyogenes
atgattaaacgctacgatgaacatcatcaagacttgactcttttaaaagctttagttcgacaacaacttccagaaaagtataaagaaatcttttttgatcaatcaaaaaacggatatgca

SEQ ID NO: 34 - DNA, 120 bp, Streptococcus pyogenes
tgattaaacgctacgatgaacatcatcaagacttgactcttttaaaagctttagttcgacaacaacttccagaaaagtataaagaaatcttttttgatcaatcaaaaaacggatatgcag

SEQ ID NO: 35 - DNA, 120 bp, Streptococcus pyogenes
ttttaaaagctttagttcgacaacaacttccagaaaagtataaagaaatcttttttgatcaatcaaaaaacggatatgcaggttatattgatgggggagctagccaagaagaattttata

SEQ ID NO: 36 - DNA, 120 bp, Streptococcus pyogenes
tttaaaagctttagttcgacaacaacttccagaaaagtataaagaaatcttttttgatcaatcaaaaaacggatatgcaggttatattgatgggggagctagccaagaagaattttataa

SEQ ID NO: 37 - DNA, 120 bp, Streptococcus pyogenes
taaagaaatcttttttgatcaatcaaaaaacggatatgcaggttatattgatgggggagctagccaagaagaattttataaatttatcaaaccaattttagaaaaaatggatggtactga

SEQ ID NO: 38 - DNA, 120 bp, Streptococcus pyogenes
aaagaaatcttttttgatcaatcaaaaaacggatatgcaggttatattgatgggggagctagccaagaagaattttataaatttatcaaaccaattttagaaaaaatggatggtactgag

SEQ ID NO: 39 - DNA, 120 bp, Streptococcus pyogenes
ggttatattgatgggggagctagccaagaagaattttataaatttatcaaaccaattttagaaaaaatggatggtactgaggaattattggcgaaactaaatcgtgaagatttgctgcgc

SEQ ID NO: 40 - DNA, 120 bp, Streptococcus pyogenes
gttatattgatgggggagctagccaagaagaattttataaatttatcaaaccaattttagaaaaaatggatggtactgaggaattattggcgaaactaaatcgtgaagatttgctgcgca

SEQ ID NO: 41 - DNA, 120 bp, Streptococcus pyogenes
aatttatcaaaccaattttagaaaaaatggatggtactgaggaattattggcgaaactaaatcgtgaagatttgctgcgcaagcaacggacctttgacaacggctctattccccatcaaa

SEQ ID NO: 42 - DNA, 120 bp, Streptococcus pyogenes
atttatcaaaccaattttagaaaaaatggatggtactgaggaattattggcgaaactaaatcgtgaagatttgctgcgcaagcaacggacctttgacaacggctctattccccatcaaat

SEQ ID NO: 43 - DNA, 120 bp, Streptococcus pyogenes
ggaattattggcgaaactaaatcgtgaagatttgctgcgcaagcaacggacctttgacaacggctctattccccatcaaattcacttgggtgagctgcatgctattctgagaagacaaga

SEQ ID NO: 44 - DNA, 120 bp, Streptococcus pyogenes
gaattattggcgaaactaaatcgtgaagatttgctgcgcaagcaacggacctttgacaacggctctattccccatcaaattcacttgggtgagctgcatgctattctgagaagacaagaa

SEQ ID NO: 45 - DNA, 120 bp, Streptococcus pyogenes
aagcaacggacctttgacaacggctctattccccatcaaattcacttgggtgagctgcatgctattctgagaagacaagaagacttttatccatttttaaaagacaatcgtgagaagatt

SEQ ID NO: 46 - DNA, 120 bp, Streptococcus pyogenes
agcaacggacctttgacaacggctctattccccatcaaattcacttgggtgagctgcatgctattctgagaagacaagaagacttttatccatttttaaaagacaatcgtgagaagattg

SEQ ID NO: 47 - DNA, 120 bp, Streptococcus pyogenes
tcacttgggtgagctgcatgctattctgagaagacaagaagacttttatccatttttaaaagacaatcgtgagaagattgaaaaaatcttgacttttcgaattccttattatgttggtcc

SEQ ID NO: 48 - DNA, 120 bp, Streptococcus pyogenes
agacttttatccatttttaaaagacaatcgtgagaagattgaaaaaatcttgacttttcgaattccttattatgttggtccattggcgcgtggcaatagtcgttttgcatggatgactcg

SEQ ID NO: 49 - DNA, 120 bp, Streptococcus pyogenes
gacttttatccatttttaaaagacaatcgtgagaagattgaaaaaatcttgacttttcgaattccttattatgttggtccattggcgcgtggcaatagtcgttttgcatggatgactcgg

SEQ ID NO: 50 - DNA, 120 bp, Streptococcus pyogenes
gaaaaaatcttgacttttcgaattccttattatgttggtccattggcgcgtggcaatagtcgttttgcatggatgactcggaagtctgaagaaacaattaccccatggaattttgaagaa

SEQ ID NO: 51 - DNA, 120 bp, Streptococcus pyogenes
aaaaaatcttgacttttcgaattccttattatgttggtccattggcgcgtggcaatagtcgttttgcatggatgactcggaagtctgaagaaacaattaccccatggaattttgaagaag

SEQ ID NO: 52 - DNA, 120 bp, Streptococcus pyogenes
cattggcgcgtggcaatagtcgttttgcatggatgactcggaagtctgaagaaacaattaccccatggaattttgaagaagttgtcgataaaggtgcttcagctcaatcatttattgaac

SEQ ID NO: 53 - DNA, 120 bp, Streptococcus pyogenes
attggcgcgtggcaatagtcgttttgcatggatgactcggaagtctgaagaaacaattaccccatggaattttgaagaagttgtcgataaaggtgcttcagctcaatcatttattgaacg

SEQ ID NO: 54 - DNA, 120 bp, Streptococcus pyogenes
gaagtctgaagaaacaattaccccatggaattttgaagaagttgtcgataaaggtgcttcagctcaatcatttattgaacgcatgacaaactttgataaaaatcttccaaatgaaaaagt

SEQ ID NO: 55 - DNA, 120 bp, Streptococcus pyogenes
aagtctgaagaaacaattaccccatggaattttgaagaagttgtcgataaaggtgcttcagctcaatcatttattgaacgcatgacaaactttgataaaaatcttccaaatgaaaaagta

SEQ ID NO: 56 - DNA, 120 bp, Streptococcus pyogenes
gttgtcgataaaggtgcttcagctcaatcatttattgaacgcatgacaaactttgataaaaatcttccaaatgaaaaagtactaccaaaacatagtttgctttatgagtattttacggtt

SEQ ID NO: 57 - DNA, 120 bp, Streptococcus pyogenes
ttgtcgataaaggtgcttcagctcaatcatttattgaacgcatgacaaactttgataaaaatcttccaaatgaaaaagtactaccaaaacatagtttgctttatgagtattttacggttt

SEQ ID NO: 58 - DNA, 120 bp, Streptococcus pyogenes
gcatgacaaactttgataaaaatcttccaaatgaaaaagtactaccaaaacatagtttgctttatgagtattttacggtttataacgaattgacaaaggtcaaatatgttactgagggaa

SEQ ID NO: 59 - DNA, 120 bp, Streptococcus pyogenes
catgacaaactttgataaaaatcttccaaatgaaaaagtactaccaaaacatagtttgctttatgagtattttacggtttataacgaattgacaaaggtcaaatatgttactgagggaat

SEQ ID NO: 60 - DNA, 120 bp, Streptococcus pyogenes
ctaccaaaacatagtttgctttatgagtattttacggtttataacgaattgacaaaggtcaaatatgttactgagggaatgcgaaaaccagcatttctttcaggtgaacagaagaaagcc

SEQ ID NO: 61 - DNA, 120 bp, Streptococcus pyogenes
ataacgaattgacaaaggtcaaatatgttactgagggaatgcgaaaaccagcatttctttcaggtgaacagaagaaagccattgttgatttactcttcaaaacaaatcgaaaagtaaccg

SEQ ID NO: 62 - DNA, 120 bp, Streptococcus pyogenes
tgcgaaaaccagcatttctttcaggtgaacagaagaaagccattgttgatttactcttcaaaacaaatcgaaaagtaaccgttaagcaattaaaagaagattatttcaaaaaaatagaat

SEQ ID NO: 63 - DNA, 120 bp, Streptococcus pyogenes
gcgaaaaccagcatttctttcaggtgaacagaagaaagccattgttgatttactcttcaaaacaaatcgaaaagtaaccgttaagcaattaaaagaagattatttcaaaaaaatagaatg

SEQ ID NO: 64 - DNA, 120 bp, Streptococcus pyogenes
cattgttgatttactcttcaaaacaaatcgaaaagtaaccgttaagcaattaaaagaagattatttcaaaaaaatagaatgttttgatagtgttgaaatttcaggagttgaagatagatt

SEQ ID NO: 65 - DNA, 120 bp, Streptococcus pyogenes
attgttgatttactcttcaaaacaaatcgaaaagtaaccgttaagcaattaaaagaagattatttcaaaaaaatagaatgttttgatagtgttgaaatttcaggagttgaagatagattt

SEQ ID NO: 66 - DNA, 120 bp, Streptococcus pyogenes
gttaagcaattaaaagaagattatttcaaaaaaatagaatgttttgatagtgttgaaatttcaggagttgaagatagatttaatgcttcattaggtacctaccatgatttgctaaaaatt

SEQ ID NO: 67 - DNA, 120 bp, Streptococcus pyogenes
ttaagcaattaaaagaagattatttcaaaaaaatagaatgttttgatagtgttgaaatttcaggagttgaagatagatttaatgcttcattaggtacctaccatgatttgctaaaaatta

SEQ ID NO: 68 - DNA, 120 bp, Streptococcus pyogenes
ttttgatagtgttgaaatttcaggagttgaagatagatttaatgcttcattaggtacctaccatgatttgctaaaaattattaaagataaagattttttggataatgaagaaaatgaaga

SEQ ID NO: 69 - DNA, 120 bp, Streptococcus pyogenes
aatgcttcattaggtacctaccatgatttgctaaaaattattaaagataaagattttttggataatgaagaaaatgaagatatcttagaggatattgttttaacattgaccttatttgaa

SEQ ID NO: 70 - DNA, 120 bp, Streptococcus pyogenes
ttaaagataaagattttttggataatgaagaaaatgaagatatcttagaggatattgttttaacattgaccttatttgaagatagggagatgattgaggaaagacttaaaacatatgctc

SEQ ID NO: 71 - DNA, 120 bp, Streptococcus pyogenes
atatcttagaggatattgttttaacattgaccttatttgaagatagggagatgattgaggaaagacttaaaacatatgctcacctctttgatgataaggtgatgaaacagcttaaacgtc

SEQ ID NO: 72 - DNA, 120 bp, Streptococcus pyogenes
tatcttagaggatattgttttaacattgaccttatttgaagatagggagatgattgaggaaagacttaaaacatatgctcacctctttgatgataaggtgatgaaacagcttaaacgtcg

SEQ ID NO: 73 - DNA, 120 bp, Streptococcus pyogenes
agatagggagatgattgaggaaagacttaaaacatatgctcacctctttgatgataaggtgatgaaacagcttaaacgtcgccgttatactggttggggacgtttgtctcgaaaattgat

SEQ ID NO: 74 - DNA, 120 bp, Streptococcus pyogenes
gatagggagatgattgaggaaagacttaaaacatatgctcacctctttgatgataaggtgatgaaacagcttaaacgtcgccgttatactggttggggacgtttgtctcgaaaattgatt

SEQ ID NO: 75 - DNA, 120 bp, Streptococcus pyogenes
cacctctttgatgataaggtgatgaaacagcttaaacgtcgccgttatactggttggggacgtttgtctcgaaaattgattaatggtattagggataagcaatctggcaaaacaatatta

SEQ ID NO: 76 - DNA, 120 bp, Streptococcus pyogenes
acctctttgatgataaggtgatgaaacagcttaaacgtcgccgttatactggttggggacgtttgtctcgaaaattgattaatggtattagggataagcaatctggcaaaacaatattag

SEQ ID NO: 77 - DNA, 120 bp, Streptococcus pyogenes
gccgttatactggttggggacgtttgtctcgaaaattgattaatggtattagggataagcaatctggcaaaacaatattagattttttgaaatcagatggttttgccaatcgcaatttta

SEQ ID NO: 78 - DNA, 120 bp, Streptococcus pyogenes
ccgttatactggttggggacgtttgtctcgaaaattgattaatggtattagggataagcaatctggcaaaacaatattagattttttgaaatcagatggttttgccaatcgcaattttat

SEQ ID NO: 79 - DNA, 120 bp, Streptococcus pyogenes
taatggtattagggataagcaatctggcaaaacaatattagattttttgaaatcagatggttttgccaatcgcaattttatgcagctgatccatgatgatagtttgacatttaaagaaga

SEQ ID NO: 80 - DNA, 120 bp, Streptococcus pyogenes
aatggtattagggataagcaatctggcaaaacaatattagattttttgaaatcagatggttttgccaatcgcaattttatgcagctgatccatgatgatagtttgacatttaaagaagac

SEQ ID NO: 81 - DNA, 120 bp, Streptococcus pyogenes
gattttttgaaatcagatggttttgccaatcgcaattttatgcagctgatccatgatgatagtttgacatttaaagaagacattcaaaaagcacaagtgtctggacaaggcgatagttta

SEQ ID NO: 82 - DNA, 120 bp, Streptococcus pyogenes
attttttgaaatcagatggttttgccaatcgcaattttatgcagctgatccatgatgatagtttgacatttaaagaagacattcaaaaagcacaagtgtctggacaaggcgatagtttac

SEQ ID NO: 83 - DNA, 120 bp, Streptococcus pyogenes
tgcagctgatccatgatgatagtttgacatttaaagaagacattcaaaaagcacaagtgtctggacaaggcgatagtttacatgaacatattgcaaatttagctggtagccctgctatta

SEQ ID NO: 84 - DNA, 120 bp, Streptococcus pyogenes
gcagctgatccatgatgatagtttgacatttaaagaagacattcaaaaagcacaagtgtctggacaaggcgatagtttacatgaacatattgcaaatttagctggtagccctgctattaa

SEQ ID NO: 85 - DNA, 120 bp, Streptococcus pyogenes
cattcaaaaagcacaagtgtctggacaaggcgatagtttacatgaacatattgcaaatttagctggtagccctgctattaaaaaaggtattttacagactgtaaaagttgttgatgaatt

SEQ ID NO: 86 - DNA, 120 bp, Streptococcus pyogenes
attcaaaaagcacaagtgtctggacaaggcgatagtttacatgaacatattgcaaatttagctggtagccctgctattaaaaaaggtattttacagactgtaaaagttgttgatgaattg

SEQ ID NO: 87 - DNA, 120 bp, Streptococcus pyogenes
catgaacatattgcaaatttagctggtagccctgctattaaaaaaggtattttacagactgtaaaagttgttgatgaattggtcaaagtaatggggcggcataagccagaaaatatcgtt

SEQ ID NO: 88 - DNA, 120 bp, Streptococcus pyogenes
atgaacatattgcaaatttagctggtagccctgctattaaaaaaggtattttacagactgtaaaagttgttgatgaattggtcaaagtaatggggcggcataagccagaaaatatcgtta

SEQ ID NO: 89 - DNA, 120 bp, Streptococcus pyogenes
aaaaaggtattttacagactgtaaaagttgttgatgaattggtcaaagtaatggggcggcataagccagaaaatatcgttattgaaatggcacgtgaaaatcagacaactcaaaagggcc

SEQ ID NO: 90 - DNA, 120 bp, Streptococcus pyogenes
aaaaggtattttacagactgtaaaagttgttgatgaattggtcaaagtaatggggcggcataagccagaaaatatcgttattgaaatggcacgtgaaaatcagacaactcaaaagggcca

SEQ ID NO: 91 - DNA, 120 bp, Streptococcus pyogenes
ggtcaaagtaatggggcggcataagccagaaaatatcgttattgaaatggcacgtgaaaatcagacaactcaaaagggccagaaaaattcgcgtgagcgtatgaaacgtattgaagaagg

SEQ ID NO: 92 - DNA, 120 bp, Streptococcus pyogenes
gtcaaagtaatggggcggcataagccagaaaatatcgttattgaaatggcacgtgaaaatcagacaactcaaaagggccagaaaaattcgcgtgagcgtatgaaacgtattgaagaaggt

SEQ ID NO: 93 - DNA, 120 bp, Streptococcus pyogenes
ttgaaatggcacgtgaaaatcagacaactcaaaagggccagaaaaattcgcgtgagcgtatgaaacgtattgaagaaggtatcaaagaattaggaagtcagattcttaaagagcatcctg

SEQ ID NO: 94 - DNA, 120 bp, Streptococcus pyogenes
gaaaaattcgcgtgagcgtatgaaacgtattgaagaaggtatcaaagaattaggaagtcagattcttaaagagcatcctgttgaaaatactcaattgcaaaatgaaaagctctatctcta

SEQ ID NO: 95 - DNA, 120 bp, Streptococcus pyogenes
atcaaagaattaggaagtcagattcttaaagagcatcctgttgaaaatactcaattgcaaaatgaaaagctctatctctattatctccaaaatggaagagacatgtatgtggaccaagaa

SEQ ID NO: 96 - DNA, 120 bp, Streptococcus pyogenes
ttgaaaatactcaattgcaaaatgaaaagctctatctctattatctccaaaatggaagagacatgtatgtggaccaagaattagatattaatcgtttaagtgattatgatgtcgatcaca

SEQ ID NO: 97 - DNA, 120 bp, Streptococcus pyogenes
ttatctccaaaatggaagagacatgtatgtggaccaagaattagatattaatcgtttaagtgattatgatgtcgatcacattgttccacaaagtttcattaaagacgattcaatagacaa

SEQ ID NO: 98 - DNA, 120 bp, Streptococcus pyogenes
ttagatattaatcgtttaagtgattatgatgtcgatcacattgttccacaaagtttcattaaagacgattcaatagacaataaggtcttaacgcgttctgataaaaatcgtggtaaatcg

SEQ ID NO: 99 - DNA, 120 bp, Streptococcus pyogenes
agatattaatcgtttaagtgattatgatgtcgatcacattgttccacaaagtttcattaaagacgattcaatagacaataaggtcttaacgcgttctgataaaaatcgtggtaaatcgga

SEQ ID NO: 100 - DNA, 120 bp, Streptococcus pyogenes
ttgttccacaaagtttcattaaagacgattcaatagacaataaggtcttaacgcgttctgataaaaatcgtggtaaatcggataacgttccaagtgaagaagtagtcaaaaagatgaaaa

SEQ ID NO: 101 - DNA, 120 bp, Streptococcus pyogenes
gttccacaaagtttcattaaagacgattcaatagacaataaggtcttaacgcgttctgataaaaatcgtggtaaatcggataacgttccaagtgaagaagtagtcaaaaagatgaaaaac

SEQ ID NO: 102 - DNA, 120 bp, Streptococcus pyogenes
taaggtcttaacgcgttctgataaaaatcgtggtaaatcggataacgttccaagtgaagaagtagtcaaaaagatgaaaaactattggagacaacttctaaatgccaagttaatcactca

SEQ ID NO: 103 - DNA, 120 bp, Streptococcus pyogenes
gataacgttccaagtgaagaagtagtcaaaaagatgaaaaactattggagacaacttctaaatgccaagttaatcactcaacgtaagtttgataatttaacgaaagctgaacgtggaggt

SEQ ID NO: 104 - DNA, 120 bp, Streptococcus pyogenes
actattggagacaacttctaaatgccaagttaatcactcaacgtaagtttgataatttaacgaaagctgaacgtggaggtttgagtgaacttgataaagctggttttatcaaacgccaat

SEQ ID NO: 105 - DNA, 120 bp, Streptococcus pyogenes
acgtaagtttgataatttaacgaaagctgaacgtggaggtttgagtgaacttgataaagctggttttatcaaacgccaattggttgaaactcgccaaatcactaagcatgtggcacaaat

SEQ ID NO: 106 - DNA, 120 bp, Streptococcus pyogenes
gtaagtttgataatttaacgaaagctgaacgtggaggtttgagtgaacttgataaagctggttttatcaaacgccaattggttgaaactcgccaaatcactaagcatgtggcacaaattt

SEQ ID NO: 107 - DNA, 120 bp, Streptococcus pyogenes
taagtttgataatttaacgaaagctgaacgtggaggtttgagtgaacttgataaagctggttttatcaaacgccaattggttgaaactcgccaaatcactaagcatgtggcacaaatttt

SEQ ID NO: 108 - DNA, 120 bp, Streptococcus pyogenes
ttgagtgaacttgataaagctggttttatcaaacgccaattggttgaaactcgccaaatcactaagcatgtggcacaaattttggatagtcgcatgaatactaaatacgatgaaaatgat

SEQ ID NO: 109 - DNA, 120 bp, Streptococcus pyogenes
gagtgaacttgataaagctggttttatcaaacgccaattggttgaaactcgccaaatcactaagcatgtggcacaaattttggatagtcgcatgaatactaaatacgatgaaaatgataa

SEQ ID NO: 110 - DNA, 120 bp, Streptococcus pyogenes
agtgaacttgataaagctggttttatcaaacgccaattggttgaaactcgccaaatcactaagcatgtggcacaaattttggatagtcgcatgaatactaaatacgatgaaaatgataaa

SEQ ID NO: 111 - DNA, 120 bp, Streptococcus pyogenes
tggttgaaactcgccaaatcactaagcatgtggcacaaattttggatagtcgcatgaatactaaatacgatgaaaatgataaacttattcgagaggttaaagtgattaccttaaaatcta

SEQ ID NO: 112 - DNA, 120 bp, Streptococcus pyogenes
gttgaaactcgccaaatcactaagcatgtggcacaaattttggatagtcgcatgaatactaaatacgatgaaaatgataaacttattcgagaggttaaagtgattaccttaaaatctaaa

SEQ ID NO: 113 - DNA, 120 bp, Streptococcus pyogenes
ttgaaactcgccaaatcactaagcatgtggcacaaattttggatagtcgcatgaatactaaatacgatgaaaatgataaacttattcgagaggttaaagtgattaccttaaaatctaaat

SEQ ID NO: 114 - DNA, 120 bp, Streptococcus pyogenes
tttggatagtcgcatgaatactaaatacgatgaaaatgataaacttattcgagaggttaaagtgattaccttaaaatctaaattagtttctgacttccgaaaagatttccaattctataa

SEQ ID NO: 115 - DNA, 120 bp, Streptococcus pyogenes
tggatagtcgcatgaatactaaatacgatgaaaatgataaacttattcgagaggttaaagtgattaccttaaaatctaaattagtttctgacttccgaaaagatttccaattctataaag

SEQ ID NO: 116 - DNA, 120 bp, Streptococcus pyogenes
ggatagtcgcatgaatactaaatacgatgaaaatgataaacttattcgagaggttaaagtgattaccttaaaatctaaattagtttctgacttccgaaaagatttccaattctataaagt

SEQ ID NO: 117 - DNA, 120 bp, Streptococcus pyogenes
aaacttattcgagaggttaaagtgattaccttaaaatctaaattagtttctgacttccgaaaagatttccaattctataaagtacgtgagattaacaattaccatcatgcccatgatgcg

SEQ ID NO: 118 - DNA, 120 bp, Streptococcus pyogenes
acttattcgagaggttaaagtgattaccttaaaatctaaattagtttctgacttccgaaaagatttccaattctataaagtacgtgagattaacaattaccatcatgcccatgatgcgta

SEQ ID NO: 119 - DNA, 120 bp, Streptococcus pyogenes
cttattcgagaggttaaagtgattaccttaaaatctaaattagtttctgacttccgaaaagatttccaattctataaagtacgtgagattaacaattaccatcatgcccatgatgcgtat

SEQ ID NO: 120 - DNA, 120 bp, Streptococcus pyogenes
aaattagtttctgacttccgaaaagatttccaattctataaagtacgtgagattaacaattaccatcatgcccatgatgcgtatcttaatgccgtcgttggaactgctttgattaagaaa

SEQ ID NO: 121 - DNA, 120 bp, Streptococcus pyogenes
aattagtttctgacttccgaaaagatttccaattctataaagtacgtgagattaacaattaccatcatgcccatgatgcgtatcttaatgccgtcgttggaactgctttgattaagaaat

SEQ ID NO: 122 - DNA, 120 bp, Streptococcus pyogenes
aagtacgtgagattaacaattaccatcatgcccatgatgcgtatcttaatgccgtcgttggaactgctttgattaagaaatatccaaaacttgaatcggagtttgtctatggtgattata

SEQ ID NO: 123 - DNA, 120 bp, Streptococcus pyogenes
agtacgtgagattaacaattaccatcatgcccatgatgcgtatcttaatgccgtcgttggaactgctttgattaagaaatatccaaaacttgaatcggagtttgtctatggtgattataa

SEQ ID NO: 124 - DNA, 120 bp, Streptococcus pyogenes
tatcttaatgccgtcgttggaactgctttgattaagaaatatccaaaacttgaatcggagtttgtctatggtgattataaagtttatgatgttcgtaaaatgcttgctaagtctgagcag

SEQ ID NO: 125 - DNA, 120 bp, Streptococcus pyogenes
atccaaaacttgaatcggagtttgtctatggtgattataaagtttatgatgttcgtaaaatgcttgctaagtctgagcaggaaataggcaaagcaaccgcaaaatatttcttttactcta

SEQ ID NO: 126 - DNA, 120 bp, Streptococcus pyogenes
agtttatgatgttcgtaaaatgcttgctaagtctgagcaggaaataggcaaagcaaccgcaaaatatttcttttactctaatatcatgaacttcttcaaaacagaaattacacttgcaaa

SEQ ID NO: 127 - DNA, 120 bp, Streptococcus pyogenes
ggaaataggcaaagcaaccgcaaaatatttcttttactctaatatcatgaacttcttcaaaacagaaattacacttgcaaatggagagattcgcaaacgccctctaatcgaaactaatgg

SEQ ID NO: 128 - DNA, 120 bp, Streptococcus pyogenes
gaaataggcaaagcaaccgcaaaatatttcttttactctaatatcatgaacttcttcaaaacagaaattacacttgcaaatggagagattcgcaaacgccctctaatcgaaactaatggg

SEQ ID NO: 129 - DNA, 120 bp, Streptococcus pyogenes
ataggcaaagcaaccgcaaaatatttcttttactctaatatcatgaacttcttcaaaacagaaattacacttgcaaatggagagattcgcaaacgccctctaatcgaaactaatggggaa

SEQ ID NO: 130 - DNA, 120 bp, Streptococcus pyogenes
aatatcatgaacttcttcaaaacagaaattacacttgcaaatggagagattcgcaaacgccctctaatcgaaactaatggggaaactggagaaattgtctgggataaagggcgagatttt

SEQ ID NO: 131 - DNA, 120 bp, Streptococcus pyogenes
atatcatgaacttcttcaaaacagaaattacacttgcaaatggagagattcgcaaacgccctctaatcgaaactaatggggaaactggagaaattgtctgggataaagggcgagattttg

SEQ ID NO: 132 - DNA, 120 bp, Streptococcus pyogenes
tcatgaacttcttcaaaacagaaattacacttgcaaatggagagattcgcaaacgccctctaatcgaaactaatggggaaactggagaaattgtctgggataaagggcgagattttgcca

SEQ ID NO: 133 - DNA, 120 bp, Streptococcus pyogenes
atggagagattcgcaaacgccctctaatcgaaactaatggggaaactggagaaattgtctgggataaagggcgagattttgccacagtgcgcaaagtattgtccatgccccaagtcaata

SEQ ID NO: 134 - DNA, 120 bp, Streptococcus pyogenes
tggagagattcgcaaacgccctctaatcgaaactaatggggaaactggagaaattgtctgggataaagggcgagattttgccacagtgcgcaaagtattgtccatgccccaagtcaatat

SEQ ID NO: 135 - DNA, 120 bp, Streptococcus pyogenes
agagattcgcaaacgccctctaatcgaaactaatggggaaactggagaaattgtctgggataaagggcgagattttgccacagtgcgcaaagtattgtccatgccccaagtcaatattgt

SEQ ID NO: 136 - DNA, 120 bp, Streptococcus pyogenes
ggaaactggagaaattgtctgggataaagggcgagattttgccacagtgcgcaaagtattgtccatgccccaagtcaatattgtcaagaaaacagaagtacagacaggcggattctccaa

SEQ ID NO: 137 - DNA, 120 bp, Streptococcus pyogenes
gaaactggagaaattgtctgggataaagggcgagattttgccacagtgcgcaaagtattgtccatgccccaagtcaatattgtcaagaaaacagaagtacagacaggcggattctccaag

SEQ ID NO: 138 - DNA, 120 bp, Streptococcus pyogenes
actggagaaattgtctgggataaagggcgagattttgccacagtgcgcaaagtattgtccatgccccaagtcaatattgtcaagaaaacagaagtacagacaggcggattctccaaggag

SEQ ID NO: 139 - DNA, 120 bp, Streptococcus pyogenes
gccacagtgcgcaaagtattgtccatgccccaagtcaatattgtcaagaaaacagaagtacagacaggcggattctccaaggagtcaattttaccaaaaagaaattcggacaagcttatt

SEQ ID NO: 140 - DNA, 120 bp, Streptococcus pyogenes
ccacagtgcgcaaagtattgtccatgccccaagtcaatattgtcaagaaaacagaagtacagacaggcggattctccaaggagtcaattttaccaaaaagaaattcggacaagcttattg

SEQ ID NO: 141 - DNA, 120 bp, Streptococcus pyogenes
cagtgcgcaaagtattgtccatgccccaagtcaatattgtcaagaaaacagaagtacagacaggcggattctccaaggagtcaattttaccaaaaagaaattcggacaagcttattgctc

SEQ ID NO: 142 - DNA, 120 bp, Streptococcus pyogenes
ttgtcaagaaaacagaagtacagacaggcggattctccaaggagtcaattttaccaaaaagaaattcggacaagcttattgctcgtaaaaaagactgggatccaaaaaaatatggtggtt

SEQ ID NO: 143 - DNA, 120 bp, Streptococcus pyogenes
tgtcaagaaaacagaagtacagacaggcggattctccaaggagtcaattttaccaaaaagaaattcggacaagcttattgctcgtaaaaaagactgggatccaaaaaaatatggtggttt

SEQ ID NO: 144 - DNA, 120 bp, Streptococcus pyogenes
caagaaaacagaagtacagacaggcggattctccaaggagtcaattttaccaaaaagaaattcggacaagcttattgctcgtaaaaaagactgggatccaaaaaaatatggtggttttga

SEQ ID NO: 145 - DNA, 120 bp, Streptococcus pyogenes
ggagtcaattttaccaaaaagaaattcggacaagcttattgctcgtaaaaaagactgggatccaaaaaaatatggtggttttgatagtccaacggtagcttattcagtcctagtggttgc

SEQ ID NO: 146 - DNA, 120 bp, Streptococcus pyogenes
gagtcaattttaccaaaaagaaattcggacaagcttattgctcgtaaaaaagactgggatccaaaaaaatatggtggttttgatagtccaacggtagcttattcagtcctagtggttgct

SEQ ID NO: 147 - DNA, 120 bp, Streptococcus pyogenes
tcaattttaccaaaaagaaattcggacaagcttattgctcgtaaaaaagactgggatccaaaaaaatatggtggttttgatagtccaacggtagcttattcagtcctagtggttgctaag

SEQ ID NO: 148 - DNA, 120 bp, Streptococcus pyogenes
gctcgtaaaaaagactgggatccaaaaaaatatggtggttttgatagtccaacggtagcttattcagtcctagtggttgctaaggtggaaaaagggaaatcgaagaagttaaaatccgtt

SEQ ID NO: 149 - DNA, 120 bp, Streptococcus pyogenes
ctcgtaaaaaagactgggatccaaaaaaatatggtggttttgatagtccaacggtagcttattcagtcctagtggttgctaaggtggaaaaagggaaatcgaagaagttaaaatccgtta

SEQ ID NO: 150 - DNA, 120 bp, Streptococcus pyogenes
gtaaaaaagactgggatccaaaaaaatatggtggttttgatagtccaacggtagcttattcagtcctagtggttgctaaggtggaaaaagggaaatcgaagaagttaaaatccgttaaag

SEQ ID NO: 151 - DNA, 120 bp, Streptococcus pyogenes
tgatagtccaacggtagcttattcagtcctagtggttgctaaggtggaaaaagggaaatcgaagaagttaaaatccgttaaagagttactagggatcacaattatggaaagaagttcctt

SEQ ID NO: 152 - DNA, 120 bp, Streptococcus pyogenes
tagtccaacggtagcttattcagtcctagtggttgctaaggtggaaaaagggaaatcgaagaagttaaaatccgttaaagagttactagggatcacaattatggaaagaagttcctttga

SEQ ID NO: 153 - DNA, 120 bp, Streptococcus pyogenes
aaggtggaaaaagggaaatcgaagaagttaaaatccgttaaagagttactagggatcacaattatggaaagaagttcctttgaaaaaaatccgattgactttttagaagctaaaggatat

SEQ ID NO: 154 - DNA, 120 bp, Streptococcus pyogenes
gtggaaaaagggaaatcgaagaagttaaaatccgttaaagagttactagggatcacaattatggaaagaagttcctttgaaaaaaatccgattgactttttagaagctaaaggatataag

SEQ ID NO: 155 - DNA, 120 bp, Streptococcus pyogenes
aagagttactagggatcacaattatggaaagaagttcctttgaaaaaaatccgattgactttttagaagctaaaggatataaggaagttagaaaagacttaatcattaaactacctaaat

SEQ ID NO: 156 - DNA, 120 bp, Streptococcus pyogenes
tgaaaaaaatccgattgactttttagaagctaaaggatataaggaagttagaaaagacttaatcattaaactacctaaatatagtctttttgagttagaaaacggtcgtaaacggatgct

SEQ ID NO: 157 - DNA, 120 bp, Streptococcus pyogenes
aaggaagttagaaaagacttaatcattaaactacctaaatatagtctttttgagttagaaaacggtcgtaaacggatgctggctagtgccggagaattacaaaaaggaaatgagctggct

SEQ ID NO: 158 - DNA, 120 bp, Streptococcus pyogenes
atagtctttttgagttagaaaacggtcgtaaacggatgctggctagtgccggagaattacaaaaaggaaatgagctggctctgccaagcaaatatgtgaattttttatatttagctagtc

SEQ ID NO: 159 - DNA, 120 bp, Streptococcus pyogenes
gtctttttgagttagaaaacggtcgtaaacggatgctggctagtgccggagaattacaaaaaggaaatgagctggctctgccaagcaaatatgtgaattttttatatttagctagtcatt

SEQ ID NO: 160 - DNA, 120 bp, Streptococcus pyogenes
ggctagtgccggagaattacaaaaaggaaatgagctggctctgccaagcaaatatgtgaattttttatatttagctagtcattatgaaaagttgaagggtagtccagaagataacgaaca

SEQ ID NO: 161 - DNA, 120 bp, Streptococcus pyogenes
tagtgccggagaattacaaaaaggaaatgagctggctctgccaagcaaatatgtgaattttttatatttagctagtcattatgaaaagttgaagggtagtccagaagataacgaacaaaa

SEQ ID NO: 162 - DNA, 120 bp, Streptococcus pyogenes
tctgccaagcaaatatgtgaattttttatatttagctagtcattatgaaaagttgaagggtagtccagaagataacgaacaaaaacaattgtttgtggagcagcataagcattatttaga

SEQ ID NO: 163 - DNA, 120 bp, Streptococcus pyogenes
ctgccaagcaaatatgtgaattttttatatttagctagtcattatgaaaagttgaagggtagtccagaagataacgaacaaaaacaattgtttgtggagcagcataagcattatttagat

SEQ ID NO: 164 - DNA, 120 bp, Streptococcus pyogenes
ccaagcaaatatgtgaattttttatatttagctagtcattatgaaaagttgaagggtagtccagaagataacgaacaaaaacaattgtttgtggagcagcataagcattatttagatgag

SEQ ID NO: 165 - DNA, 120 bp, Streptococcus pyogenes
cattatgaaaagttgaagggtagtccagaagataacgaacaaaaacaattgtttgtggagcagcataagcattatttagatgagattattgagcaaatcagtgaattttctaagcgtgtt

SEQ ID NO: 166 - DNA, 120 bp, Streptococcus pyogenes
attatgaaaagttgaagggtagtccagaagataacgaacaaaaacaattgtttgtggagcagcataagcattatttagatgagattattgagcaaatcagtgaattttctaagcgtgtta

SEQ ID NO: 167 - DNA, 120 bp, Streptococcus pyogenes
atgaaaagttgaagggtagtccagaagataacgaacaaaaacaattgtttgtggagcagcataagcattatttagatgagattattgagcaaatcagtgaattttctaagcgtgttattt

SEQ ID NO: 168 - DNA, 120 bp, Streptococcus pyogenes
aaaaacaattgtttgtggagcagcataagcattatttagatgagattattgagcaaatcagtgaattttctaagcgtgttattttagcagatgccaatttagataaagttcttagtgcat

SEQ ID NO: 169 - DNA, 120 bp, Streptococcus pyogenes
aaaacaattgtttgtggagcagcataagcattatttagatgagattattgagcaaatcagtgaattttctaagcgtgttattttagcagatgccaatttagataaagttcttagtgcata

SEQ ID NO: 170 - DNA, 120 bp, Streptococcus pyogenes
acaattgtttgtggagcagcataagcattatttagatgagattattgagcaaatcagtgaattttctaagcgtgttattttagcagatgccaatttagataaagttcttagtgcatataa

SEQ ID NO: 171 - DNA, 120 bp, Streptococcus pyogenes
tgagattattgagcaaatcagtgaattttctaagcgtgttattttagcagatgccaatttagataaagttcttagtgcatataacaaacatagagacaaaccaatacgtgaacaagcaga

SEQ ID NO: 172 - DNA, 120 bp, Streptococcus pyogenes
gagattattgagcaaatcagtgaattttctaagcgtgttattttagcagatgccaatttagataaagttcttagtgcatataacaaacatagagacaaaccaatacgtgaacaagcagaa

SEQ ID NO: 173 - DNA, 120 bp, Streptococcus pyogenes
attattgagcaaatcagtgaattttctaagcgtgttattttagcagatgccaatttagataaagttcttagtgcatataacaaacatagagacaaaccaatacgtgaacaagcagaaaat

SEQ ID NO: 174 - DNA, 120 bp, Streptococcus pyogenes
attttagcagatgccaatttagataaagttcttagtgcatataacaaacatagagacaaaccaatacgtgaacaagcagaaaatattattcatttatttacgttgacgaatcttggagct

SEQ ID NO: 175 - DNA, 120 bp, Streptococcus pyogenes
ttttagcagatgccaatttagataaagttcttagtgcatataacaaacatagagacaaaccaatacgtgaacaagcagaaaatattattcatttatttacgttgacgaatcttggagctc

SEQ ID NO: 176 - DNA, 120 bp, Streptococcus pyogenes
taacaaacatagagacaaaccaatacgtgaacaagcagaaaatattattcatttatttacgttgacgaatcttggagctcccactgcttttaaatattttgatacaacaattgatcgtaa

SEQ ID NO: 177 - DNA, 120 bp, Streptococcus pyogenes
aatattattcatttatttacgttgacgaatcttggagctcccactgcttttaaatattttgatacaacaattgatcgtaaacgatatacgtctacaaaagaagttttagatgccactttt

SEQ ID NO: 178 - DNA, 120 bp, Streptococcus pyogenes
ccactgcttttaaatattttgatacaacaattgatcgtaaacgatatacgtctacaaaagaagttttagatgccacttttatccatcaatccatcactggtctttatgaaacacgcattg

SEQ ID NO: 179 - DNA, 120 bp, Streptococcus pyogenes
acaattgatcgtaaacgatatacgtctacaaaagaagttttagatgccacttttatccatcaatccatcactggtctttatgaaacacgcattgatttgagtcagctaggaggtgactga